(19) United States
(12) Patent Application Publication
(10) Pub. No.: US 2018/0207191 A1
(43) Pub. Date: Jul. 26, 2018

(54) HUMAN CANCER MICRO-RNA EXPRESSION PROFILES PREDICTIVE OF CHEMO-RESPONSE

(71) Applicant: H. LEE MOFFITT CANCER CENTER AND RESEARCH INSTITUTE, INC., Tampa, FL (US)

(72) Inventors: JOHNATHAN MARK LANCASTER, PARK CITY, UT (US); YIN XIONG, TAMPA, FL (US); NING CHEN, TAMPA, FL (US)

(21) Appl. No.: 15/675,743

(22) Filed: Aug. 13, 2017

Related U.S. Application Data
(63) Continuation of application No. 13/634,028, filed on Nov. 15, 2012, now abandoned.
(60) Provisional application No. 61/312,941, filed on Mar. 11, 2010.

(51) Int. Cl. A61K 31/7088; C12N 15/11; C12Q 1/6886

(52) U.S. Cl. CPC A61K 31/7088; C12Q 2600/178; C12Q 2600/158; C12Q 2600/136; C12Q 2600/106; C12Q 1/6886; C12N 2320/10; C12N 2310/141; C12N 15/111

(57) ABSTRACT

Disclosed are identified and successfully targeted microRNAs (miRNAs) associated with human cancer cell line response to a range of anti-cancer agents. The strategy of integrating in vitro miRNA expression and drug sensitivity data not only aid in the characterization of determinants of cytotoxic response, but also in the identification of novel therapeutic targets.

### CROSS-REFERENCE TO RELATED APPLICATIONS

The present application is a continuation of U.S. application Ser. No. 13/634,028, filed Nov. 15, 2012, which is the U.S. National Stage of International Application Number PCT/US2011/028238, filed Mar. 11, 2011, which claims the benefit of U.S. Provisional Application Ser. No. 61/312,941, filed Mar. 11, 2010, each of which is hereby incorporated by reference herein in its entirety, including any figures, tables, nucleic acid sequences, amino acid sequences, and drawings.

The Sequence Listing for this application is labeled “SeqList-12Aug17.txt” which was created on Aug. 12, 2017 and is 24 KB. The entire contents of the sequence listing is incorporated herein by reference in its entirety.

### BACKGROUND OF THE INVENTION

MicroRNAs (miRNAs) are non-coding, 21-25 nucleotide, regulatory RNAs that affect the stability and/or translational efficiency of messenger-RNA (mRNAs) [1]. It has been predicted that thousands of miRNAs exist in the human genome [2]. To data, more than 850 human miRNA genes have been identified targeting more than 34,000 mRNA genes [www.sanger.ac.uk]. Deregulation of miRNAs has been implicated in the development of many human cancers [3, 4] suggesting that some miRNAs function as tumor suppressor genes [5, 6]. For example, loss of let-7 may influence the development of lung cancer as it negatively regulates let-60/RAS [7]; miRs-34a-c play an important role in the tumor suppressor function of p53, which may also control their expression [8, 9] and miR-181a is found to be related to morphological sub-class of acute myeloid leukemia [10]. Furthermore, some data suggest that the deregulation of miRNAs appear important not only in cancer development, but also in resistance to therapy [11-15]. For example, it has been shown that miR-221/222 over-expression reduces p27(Kip1) levels and induces tamoxifen resistance due to cell cycle inhibition [12]. Similarly, the inhibition of miR-21 down-regulates Bcl-2 protein and increases apoptosis and drug sensitivity [11].

### BRIEF SUMMARY OF THE INVENTION

Few successful therapeutic options exist for patients with persistent or recurrent cancer. This is due in large part to an incomplete understanding of the molecular determinants of chemotherapy-response. Recently, it has been shown that microRNAs (miRNAs) influence messenger-RNA (mRNA) transcriptional control and can contribute to human carcinogenesis. The objective of the current study was to identify miRNAs associated with human cancer cell line response to chemotherapy, and to evaluate these miRNAs as therapeutic targets.

The expression of 435 unique miRNAs was measured in 40 solid tumor cancer cell lines selected from the NCI cancer cell line panel. miRNA expression data was integrated with publicly available chemo-sensitivity (GI50) data for each of the 40 cell lines to doxorubicin, paclitaxel, topotecan, gemcitabine, docetaxel, cisplatin and carboplatin. Analysis of miRNA expression and GI50 chemosensitivity data using Limma and SAM software, identified several miRNAs associated with cell line drug response. These miRNAs were subject to targeted expression modification and the effect on in vitro cell chemo-sensitivity evaluated using luminescent cell viability assays.

Pearson's correlation identified 22 miRNAs associated with in vitro cisplatin response (p<0.05), 48 miRNAs associated with doxorubicin response (p<0.05), 35 miRNAs associated with response to paclitaxel, topotecan (p<0.05), 34 miRNAs associated with response to gemcitabine (p<0.05), and 32 miRNAs associated with docetaxel response (p<0.05). Expression of 16 specific miRNAs (miR367, miR200c, miR515, miR377, miR508, miR340, miR129, miR130a, miR142_5p, miR155, miR296, miR34c, miR367, miR380_5p, miR489, miR494, and miR526) were associated with in vitro sensitivity to 3 or more drugs. Expression of miR-367, was most highly correlated with topotecan-sensitivity (down-regulated in topotecan resistant cell lines). Transient transfection of 786-0 and TK-10 renal cancer cells with pre-miR-367 produced an increase in topotecan-induced cell death (p<0.05).

The inventors have identified and successfully targeted miRNAs associated with human cancer cell line response to a range of cytotoxic agents. The inventors' strategy of integrating in vitro miRNA expression and drug sensitivity data may not only aid in the characterization of determinants of cytotoxic response, but also in the identification of novel therapeutic targets.

The invention provides biomarkers (expression profiles) based on the expression of miRNAs determined to be associated with response to anti-cancer agents. These biomarkers can be used to discriminate between cancers that are sensitive and resistance to anti-cancer agents such as cytotoxic agents, and are themselves therapeutic targets. The present invention relates to the use of differential miRNA expression to obtain miRNA expression profiles for cancer patients, which may be used for selecting cancer treatments with a higher likelihood of effectiveness. By predicting the cancer's sensitivity or resistance to candidate therapeutic agents, the miRNA expression profiles of the invention provide information that can be used to guide individualized cancer treatment.

One aspect of the invention provides a method for preparing a miRNA expression profile for a cancer cell sample that is indicative of resistance or sensitivity to an anti-cancer agent, comprising: determining the level of expression of an miRNA in the sample, thereby preparing the miRNA expression profile. In some embodiments, the miRNA used for the expression profile comprises one or more miRNAs listed in FIGS. 4A, 4B, 4C, 4D, 4E, 4F, or 4G. In some embodiments, the miRNA used for the expression profile comprises one or more miRNAs from among miR367, miR200c, miR515, miR377, miR508, miR340, miR129, miR130a, miR142_5p, miR155, miR296, miR34c, miR367, miR380_5p, miR489, miR494, and miR526.

Another aspect of the invention concerns a method of treating cancer in a mammalian subject, wherein the cancer has been pre-determined to express an miRNA at a level that is indicative of sensitivity, or lack of resistance, to an anti-cancer agent, wherein the method comprises administering a therapeutically effective amount of the anti-cancer agent to the subject. In some embodiments, the miRNA comprises one or more miRNAs listed in FIGS. 4A, 4B, 4C, 4D, 4E, 4F, or 4G. In some embodiments, the miRNA used for the expression profile comprises one or more miRNAs listed in FIGS. 4A, 4B, 4C, 4D, 4E, 4F, or 4G. In some embodiments, the miRNA used for the expression profile comprises one or more miRNAs from among miR367, miR200c, miR515, miR377, miR508, miR340, miR129, miR130a, miR142_5p, miR155, miR296, miR34c, miR367, miR380_5p, miR489, miR494, and miR526.

In some embodiments, the miRNA comprises one or more miRNAs listed in FIG. 4A, and wherein the anti-cancer agent comprises cisplatin or a cisplatin variant. In some embodiments, the miRNA comprises one or more miRNAs listed in FIG. 4B, and wherein the anti-cancer agent comprises docetaxel or a docetaxel variant. In some embodiments, the miRNA comprises one or more miRNAs listed in FIG. 4C, and wherein the anti-cancer agent comprises doxorubicin or a doxorubicin variant. In some embodiments, the miRNA comprises one or more miRNAs listed in FIG. 4D, and wherein the anti-cancer agent comprises gemcitabine or a gemcitabine variant. In some embodiments, the miRNA comprises one or more miRNAs listed in FIG. 4E, and wherein the anti-cancer agent comprises paclitaxel or a paclitaxel variant. In some embodiments, the miRNA comprises one or more miRNAs listed in FIG. 4F, and wherein the anti-cancer agent comprises topotecan or a topetecan variant. In some embodiments, the miRNA comprises one or more miRNAs listed in FIG. 4G, and wherein the anti-cancer agent comprises carboplatin or a carboplatin variant.

In some embodiments, the anti-cancer agent comprises one or more agents from among topotecan or a topotecan variant, paclitaxel or a paclitaxel variant, or docetaxel or a docetaxel variant, or a combination of two or more of the foregoing.

In some embodiments, the cancer comprises one or more cancers from among lung cancer, colon cancer, breast cancer, renal cancer, skin cancer, prostate cancer, cancer of the central nervous system (CNS), and hematologic cancer. In some embodiments, the cancer comprises a gynecological cancer, for example, ovarian cancer.

Another aspect of the invention includes a method for predicting the response of a cancer in a mammalian subject to an anti-cancer agent, comprising: determining the miRNA expression profile in a cancer cell sample obtained from the subject; comparing the miRNA expression profile of the cancer cell sample to a reference miRNA expression profile associated with a predetermined sensitivity or lack of resistance to one more anti-cancer agents; and determining the predicted response of the cancer cells in the cancer cell sample to the one or more anti-cancer agents based upon the compared miRNA expression profiles, wherein the predicted response of the cancer cells in the cancer cell sample is indicative of the response of the cancer in the subject. In some embodiments, the reference miRNA expression profile is the miRNA expression profile of one or more cancer cells with predetermined sensitivities to one or more anti-cancer agents. In some embodiments, the miRNA of the miRNA expression profiles comprises one or more miRNAs listed in FIGS. 4A, 4B, 4C, 4D, 4E, 4F, or 4G. In some embodiments, the miRNA of the miRNA expression profiles comprises one or more miRNAs from among miR367, miR200c, miR515, miR377, miR508, miR340, miR129, miR130a, miR142_5p, miR155, miR296, miR34c, miR367, miR380_5p, miR489, miR494, and miR526. In some embodiments, the miRNA of the miRNA expression profiles comprises one or more miRNAs listed in FIG. 4A, and wherein the anti-cancer agent comprises cisplatin or a cisplatin variant. In some embodiments, the miRNA of the miRNA expression profiles comprises one or more miRNAs listed in FIG. 4B, and wherein the anti-cancer agent comprises docetaxel or a docetaxel variant. In some embodiments, the miRNA of the miRNA expression profiles comprises one or more miRNAs listed in FIG. 4C, and wherein the anti-cancer agent comprises doxorubicin or a doxorubicin variant. In some embodiments, the miRNA of the miRNA expression profiles comprises one or more miRNAs listed in FIG. 4D, and wherein the anti-cancer agent comprises gemcitabine or a gemcitabine variant. In some embodiments, the miRNA of the miRNA expression profiles comprises one or more miRNAs listed in FIG. 4E, and wherein the anti-cancer agent comprises paclitaxel or a paclitaxel variant. In some embodiments, the miRNA of the miRNA expression profiles comprises one or more miRNAs listed in FIG. 4F, and wherein the anti-cancer agent comprises topotecan or a topetecan variant. In some embodiments, the miRNA of the miRNA expression profiles comprises one or more miRNAs listed in FIG. 4G, and wherein the anti-cancer agent comprises carboplatin or a carboplatin variant. In some embodiments, the cancer in the subject comprises one or more cancers from among lung cancer, colon cancer, breast cancer, ovarian cancer, renal cancer, skin cancer, prostate cancer, cancer of the central nervous system (CNS), and hematologic cancer. In some embodiments, the cancer in the subject and the one or more cancer cells are the same cancer type. In some embodiments, the one or more cancer cells with predetermined sensitivities comprise a primary cancer cell. In some embodiments, the one or more cancer cells with predetermined sensitivities comprise a cell of a cancer cell line. In some embodiments, the cancer cell line is a cancer type represented by the cell lines in FIG. 3 or FIGS. 5-30 (for example, lung cancer, colon cancer, breast cancer, ovarian cancer, renal cancer, melanoma, prostate, cancer of the CNS or leukemia). In some embodiments, the cancer cell line comprises two or more cancer lines of single cancer type (for example, two or more lung cancer cell lines, such as NCI-H460 and NCI-H522, or two or more ovarian cancer cell lines, such as OVCAR-8 and OVCAR-4). In some embodiments, the cancer cell line comprises one or more cancer cell lines listed in FIG. 3 or FIGS. 5-30 (e.g., NCI-H460, NCI-H522, NCI-H322M, HOP62, A549, EKVX, MALME-3M, NCI-H226, HT29, HCT-116, SE-620, HCT-15, HCC2998, COL0205, HS-578T, NCI/ADR-RES, OVCAR-8, OVCAR-4, ACHN, SN-12C, 786-O, CAKI-1, UO-31, TK-10, A498, SK-MEL-28, UACC-257, M14, UACC-62, SK-MEL-2, LOX-IMVI, DU-145, PC-3, SF-295, SF-539, SNB-75, U251, HL-60, RPMI8226, and K562).

Another aspect of the invention concerns a method for selecting a cancer treatment for a mammalian subject having cancer, comprising: determining the miRNA expression profile in a cancer cell sample obtained from the subject; comparing the miRNA expression profile of the cancer cell sample to a reference miRNA expression profile associated with a predetermined sensitivity or lack of resistance to one more anti-cancer agents; determining the predicted response of the cancer cells in the cancer cell sample to the one or more anti-cancer agents based upon the compared miRNA expression profiles, wherein the predicted response of the cancer cells in the cancer cell sample is indicative of the response of the cancer in the subject; and selecting an anti-cancer agent among the one or more anti-cancer agents associated with a predetermined sensitivity or lack of resistance for treatment of the subject. In some embodiments, the treatment selection method further comprises administering a therapeutically effective amount of the selected anti-cancer agent to the subject.

Another aspect of the invention concerns a method for screening for agents that modulate sensitivity or resistance of cancer cells to anti-cancer agents, comprising administering a candidate agent to the cancer cells in vitro or in vivo, and determining whether the candidate agent modulates the level of one or more miRNAs in the cancer cells, wherein modulation of miRNA level is indicative of modulation of sensitivity or resistance. In some embodiments, the one or more miRNAs comprise one or more miRNAs listed in FIGS. 4A, 4B, 4C, 4D, 4E, 4F, or 4G. In some embodiments, the one or more miRNAs comprise one or more miRNAs from among miR367, miR200c, miR515, miR377, miR508, miR340, miR129, miR130a, miR142_5p, miR155, miR296, miR34c, miR367, miR380_5p, miR489, miR494, and miR526.

Another aspect of the invention concerns a method for increasing the sensitivity of a cancer cell to an anti-cancer agent, comprising administering in vitro or in vivo an effective amount of an agent that inhibits or decreases the level or activity of one or more miRNAs in the cancer cells, wherein an increase of the miRNA is associated with resistance to the anti-cancer agent, and wherein administering the agent increases the sensitivity of the cancer cell to the anti-cancer agent. In some embodiments, the one or more miRNAs comprise one or more miRNAs listed in FIGS. 4A, 4B, 4C, 4D, 4E, 4F, or 4G. In some embodiments, the method further comprises administering an effective amount of the anti-cancer agent to the sensitized cancer cell in vitro or in vivo. In some embodiments, the agent that inhibits or decreases the level of one or more miRNAs comprises one or more among an anti-miRNA oligonucleotide (AMO), multiple-target AMO (MT-AMO), miRNA sponge, miRNA masking antisense oligonucleotide, and miRNA knockout agent. In some embodiments, the agent that inhibits or decreases the level of one or more miRNA comprises an antisense oligonucleotide (ASO) having a backbone modification or 2′ sugar modification selected from 2′-O-methyl (2′-O-Me), 2′-O-methoxyethyl (2′-MOE), 2′-fluoro (2′F), or locked nucleic acid (LNA). In some embodiments, the agent that inhibits or decreases the level of one or more miRNAs comprises an antagomir (anti-mRNA oligonucleotide (AMO) conjugated with cholesterol). The agent that inhibits or decreases the level or activity of one or more miRNAs in the cancer cells may be administered to a subject systemically or locally at the site of the cancer cells.

Another aspect of the invention concerns a method for increasing the sensitivity of a cancer cell to an anti-cancer agent, comprising administering in vitro or in vivo an effective amount of an agent that increases the level or activity of one or more miRNAs in the cancer cells, wherein a decrease of the miRNA is associated with resistance to the anti-cancer agent, and wherein administering the agent increases the sensitivity of the cancer cell to the anti-cancer agent. In some embodiments, the one or more miRNAs comprise one or more miRNAs listed in FIGS. 4A, 4B, 4C, 4D, 4E, 4F, or 4G. In some embodiments, the agent that increases the level of one or more miRNA in the cancer cells is an miRNA precursor (also referred to as a pre-microRNA or pre-miRNA). The agent that increases the level or activity of one or more miRNAs in the cancer cells may be administered to a subject systemically or locally at the site of the cancer cells.

In some embodiments, the subject has been diagnosed with the cancer or a pre-malignancy at the time the sample is obtained from the subject for assessment of clinical response to an anti-cancer agent (i.e., resistance/sensivity). The diagnosis of cancer or pre-malignancy may be made, for example, based on clinical parameters known to those skilled in the art for the particular disorder (e.g., diagnostic imaging procedure such as computerized tomography (CT) scan, magnetic resonance imaging (MM), and nuclear medicine (NM) imaging; biopsy and pathology report, etc.), differential miRNA expression, or a combination thereof. In other embodiments, the subject has not yet been diagnosed with the cancer or a pre-malignancy at the time the sample is obtained from the subject.

One or more cancer cell samples may be obtained from a subject by techniques known in the art, such as biopsy. The type of biopsy utilized is dependent upon the anatomical location from which the sample is to be obtained. Examples include fine needle aspiration (FSA), excisional biopsy, incisional biopsy, colonoscopic biopsy, punch biopsy, and bone marrow biopsy.

The prognostic and therapeutic methods of the invention may include adjunctive cancer treatments. Cancer treatments vary with the type of cancer to be treated. Cancer treatments most commonly used include surgery, chemotherapy, radiation treatment, or a combination of two or more of these treatments. Less commonly used treatments for cancer include laser treatment, hyperthermia, and cryosurgery. Other cancer treatments may be utilized.

Another aspect of the invention concerns computer system for performing any of the methods disclosed herein.

Another aspect of the invention concerns a probe array or probe set (an miRNA probe array) for performing any of the methods disclosed herein, comprising a plurality of probes that hybridize to 1, 2, 3, 4, 5, 6, 7, 8, 9, 10, 11, 12, 13, 14, 15, 16, 17, or more miRNAs listed in Table 1, FIGS. 4A-4G, or SEQ ID NOs:1-157. In some embodiments of the probe array or set, the miRNA comprises 1, 2, 3, 4, 5, 6, 7, 8, 9, 10, 11, 12, 13, 14, 15, 16, or 17 miRNAs from among miR367, miR200c, miR515, miR377, miR508, miR340, miR129, miR130a, miR142_5p, miR155, miR296, miR34c, miR367, miR380_5p, miR489, miR494, and miR526.

Another aspect of the invention is a kit for performing any of the methods disclosed herein, comprising the probe array or probe set of the invention.

Another aspect of the invention concerns an isolated precursor microRNA (pre-miRNA) that increases the level of one or more miRNAs from among those listed in FIGS. 4A-4G. In some embodiments, the one or more miRNAs are selected from among miR367, miR200c, miR515, miR377, miR508, miR340, miR129, miR130a, miR142_5p, miR155, miR296, miR34c, miR367, miR380_5p, miR489, miR494, and miR526.

Another aspect of the invention concerns an isolated anti-microRNA (anti-miRNA) that inhibits or decreases the level of one or more miRNAs from among those listed in FIGS. 4A-4G. In some embodiments, the one or more miRNAs are selected from among miR367, miR200c, miR515, miR377, miR508, miR340, miR129, miR130a, miR142_5p, miR155, miR296, miR34c, miR367, miR380_5p, miR489, miR494, and miR526. In some embodiments, the anti-miRNA comprises one or more among an anti-miRNA oligonucleotide (AMO), multiple-target AMO (MT-AMO), miRNA sponge, miRNA masking antisense oligonucleotide, and miRNA knockout agent. In some embodiments, the anti-miRNA comprises an antisense oligonucleotide (ASO) having a backbone modification or 2′ sugar modification selected from 2′-O-methyl (2′-O-Me), 2′-O-methoxyethyl (2′-MOE), 2′-fluoro (2′F), or locked nucleic acid (LNA). In some embodiments, the anti-miRNA comprises an antagomir (anti-mRNA oligonucleotide (AMO) conjugated with cholesterol).

In the aforementioned methods and compositions (kits, probe arrays/sets, computer systems) of the invention, the cancer cell sample from which miRNA expression information is obtained may contain, for example, primary cancer cells, or cells of a cancer cell line. In some embodiments, the cancer cell sample comprises cancer cells that have been previously determined to be resistant or sensitive to an anti-cancer agent of interest. In some embodiments, the cancer cell sample comprises cancer cells that have not been previously determined to be resistant or sensitive to the anti-cancer agent.

In some embodiments of the aforementioned methods and compositions (kits, probe arrays/sets, computer systems) of the invention, the anti-cancer agent is a cytotoxic agent. In some embodiments, the anti-cancer agent comprises one or more cytotoxic agents from among doxorubicin, paclitaxel, topotecan, gemcitabine, docetaxel, cisplatin and caraboplatin, or a variant of any of the foregoing. In some embodiments, the anti-cancer agent comprises one or more from among a platinum compound, DNA repair inhibitor, angiogenesis inhibitor, or PI3 kinase inhibitor.

In some embodiments of the aforementioned methods and compositions of the invention, the one or more miRNAs are selected from those in FIG. 4A: hsa_miR_340* (decreased), hsa_miR_512_5p (decreased), hsa_miR_26b (increased), hsa_miR_181b (decreased), hsa_miR_138 (increased), hsa_miR_342 (decreased), hsa_miR_27a (decreased), hsa_miR_181a (decreased), hsa_miR_146b (increased), hsa_miR_524 (decreased), hsa_miR_126 AS (decreased), hsa_miR_200c* (increased), hsa_miR_494* (decreased), hsa_let_7c (decreased), hsa_miR_147 (increased), hsa_miR_518a (increased), hsa_miR_296* (decreased), hsa_miR_129* (increased), hsa_miR_155* (decreased), hsa_miR_302c (increased), hsa_miR_379 (decreased), hsa_miR_523 (decreased). In some embodiments, the reference miRNA expression profile is that recited parenthetically after the aforementioned miRNAs (increased or decreased) and is indicative of the reference cell's resistance to the anti-cancer agent in question.

In some embodiments of the aforementioned methods and compositions of the invention, the one or more miRNAs are selected from those in FIG. 4B: hsa_miR_29c (decreased), hsa_miR_515_3p* (increased), hsa_miR_367* (decreased), hsa_miR_32 (decreased), hsa_miR_452 AS (decreased), hsa_miR_489* (increased), hsa_miR_1 (increased), hsa_miR_141 (decreased), hsa_miR_30a_5p (increased), hsa_miR_380_5p* (increased), hsa_miR_30a_3p (increased), hsa_miR_126 (decreased), hsa_miR_134 (increased), hsa_miR_338 (increased), hsa_miR_508* (decreased), hsa_miR_199b (decreased), hsa_miR_515_5p (increased), hsa_miR_181d (decreased), hsa_miR_520c (increased), hsa_miR_377* (decreased), hsa_miR_106a (decreased), hsa_let_7e (increased), hsa_miR_34c* (increased), hsa_miR_337 (decreased), hsa_miR_526a* (increased), hsa_miR_219 (increased), hsa_miR_30c (increased), hsa_miR_124a (increased), hsa_miR_516_3p (increased), hsa_miR_373 (increased), hsa_miR_195 (decreased), hsa_miR_302a (increased). In some embodiments, the reference miRNA expression profile is that recited parenthetically after the aforementioned miRNAs (increased or decreased) and is indicative of the reference cell's resistance to the anti-cancer agent in question.

In some embodiments of the aforementioned methods and compositions of the invention, the one or more miRNAs are selected from those in FIG. 4C: hsa_miR_504 (decreased), hsa_let_7f (increased), hsa_miR_324_3p (decreased), hsa_miR_138 (decreased), hsa_miR_30d (decreased), hsa_miR_205 (decreased), hsa_miR_378 (decreased), hsa_miR_521 (increased), hsa_miR_15b (decreased), hsa_miR_380_5p* (decreased), hsa_miR_302a (decreased), hsa_miR_491 (decreased), hsa_miR_296* (decreased), hsa_miR_423 (decreased), hsa_miR_432 (increased), hsa_let_7d (increased), hsa_miR_222 (increased), hsa_miR_425 (increased), hsa_miR_199a_AS (increased), hsa_miR_18a (decreased), hsa_miR_25 (decreased), hsa_let_7a (increased), hsa_miR_216 (decreased), hsa_miR_30b (increased), hsa_miR_154 (increased), hsa_miR_525 (increased), hsa_miR_221 (increased), hsa_miR_377* (decreased), hsa_miR_485_3p (decreased), hsa_miR_339 (decreased), hsa_miR_183 (decreased), hsa_miR_410 (decreased), hsa_miR_28 (increased), hsa_miR_142_3p (decreased), hsa_miR_361 (decreased), hsa_miR_15a (decreased), hsa_miR_1 (increased), hsa_miR_93 (decreased), hsa_miR_133b (decreased), hsa_miR_31 (increased), hsa_miR_382 (increased), hsa_miR_92 (decreased), hsa_miR_184 (decreased), hsa_miR_142_5p* (decreased), hsa_miR_508* (decreased), hsa_miR_422a (decreased), hsa_let_7b (increased), hsa_miR_103 (decreased). In some embodiments, the reference miRNA expression profile is that recited parenthetically after the aforementioned miRNAs (increased or decreased) and is indicative of the reference cell's resistance to the anti-cancer agent in question.

In some embodiments of the aforementioned methods and compositions of the invention, the one or more miRNAs are selected from those in FIG. 4D: hsa_miR_155* (decreased), hsa_miR_498 (decreased), hsa_miR_190 (increased), hsa_miR_340* (decreased), hsa_miR_213 (decreased), hsa_miR_494* (decreased), hsa_miR_526a* (increased), hsa_miR_508* (decreased), hsa_miR_154 (increased), hsa_miR_142_5p* (decreased), hsa_miR_515_3p* (increased), hsa_miR_148b (increased), hsa_miR_373 (increased), hsa_miR_19a (increased), hsa_miR_489* (decreased), hsa_miR_302c (increased), hsa_miR_146a (increased), hsa_miR_518f (increased), hsa_miR_429 (decreased), hsa_miR_524 (decreased), hsa_miR_200c* (increased), hsa_miR_130a* (decreased), hsa_miR_129* (increased), hsa_miR_224 (increased), hsa_miR_324_5p (increased), hsa_miR_181b (decreased), hsa_miR_34c* (increased), hsa_miR_520a AS (decreased), hsa_miR_381 (decreased), hsa_miR_380_5p* (increased), hsa_miR_488 (increased), hsa_miR_370 (decreased), hsa_miR_181a (decreased), hsa_miR_30b (decreased). In some embodiments, the reference miRNA expression profile is that recited parenthetically after the aforementioned miRNAs (increased or decreased) and is indicative of the reference cell's resistance to the anti-cancer agent in question.

In some embodiments of the aforementioned methods and compositions of the invention, the one or more miRNAs are selected from those in FIG. 4E: hsa_miR_367* (decreased), hsa_miR_30a_5p (increased), hsa_miR_141 (decreased), hsa_miR_30a_3p (increased), hsa_miR_516_3p (increased), hsa_miR_377* (decreased), hsa_miR_134 (increased), hsa_miR_142_5p (decreased), hsa_let_7e (increased), hsa_miR_29c (decreased), hsa_miR_218 (decreased), hsa_miR_17_3p (decreased), hsa_miR_17_5p (decreased), hsa_miR_130a* (increased), hsa_miR_195 (decreased), hsa_miR_99b (increased), hsa_miR_338 (increased), hsa_miR_106a (decreased), hsa_miR_193b (increased), hsa_miR_515_3p* (increased), hsa_miR_374 (increased), hsa_miR_125a (increased), hsa_miR_192 (decreased), hsa_miR_30c (increased), hsa_miR_95 (decreased), hsa_miR_452 AS (decreased), hsa_miR_489* (increased), hsa_miR_32 (decreased), hsa_miR_373* (increased), hsa_miR_130b (decreased), hsa_miR_19b (decreased), hsa_miR_126 (decreased), hsa_miR_148a (decreased), hsa_miR_376b (increased), hsa_miR_7 (increased). In some embodiments, the reference miRNA expression profile is that recited parenthetically after the aforementioned miRNAs (increased or decreased) and is indicative of the reference cell's resistance to the anti-cancer agent in question.

In some embodiments of the aforementioned methods and compositions of the invention, the one or more miRNAs are selected from those in FIG. 4F: hsa_miR_340* (decreased), hsa_miR_367* (decreased), hsa_miR_515_5p (increased), hsa_miR_518f (increased), hsa_miR_10a (decreased), hsa_miR_130a* (decreased), hsa_miR_30e_5p (increased), hsa_miR_508* (decreased), hsa_miR_515_3p* (increased), hsa_miR_384 (increased), hsa_miR_494* (decreased), hsa_miR_302c (increased), hsa_miR_505 (decreased), hsa_let_7g (increased), hsa_miR_155* (decreased), hsa_miR_432 AS (increased), hsa_miR_526a* (increased), hsa_miR_129* (increased), hsa_miR_324_5p (increased), hsa_miR_153 (decreased), hsa_miR_107 (increased), hsa_miR_200c* (increased), hsa_miR_10b (decreased), hsa_miR_383 (increased), hsa_miR_34c* (increased), hsa_miR_25 (increased), hsa_miR_432 (increased), hsa_miR_374 (increased), hsa_miR_518e (increased), hsa_miR_196a (decreased), hsa_miR_21 (decreased), hsa_miR_296* (decreased), hsa_miR_377* (increased), hsa_miR_518c (increased), hsa_miR_512_5p (decreased). In some embodiments, the reference miRNA expression profile is that recited parenthetically after the aforementioned miRNAs (increased or decreased) and is indicative of the reference cell's resistance to the anti-cancer agent in question.

In some embodiments of the aforementioned methods and compositions of the invention, the one or more miRNAs are selected from those in FIG. 4G: hsa_miR_342 (decreased), hsa_miR_182 (increased), hsa_miR_181b (decreased), hsa_miR_181a (decreased), hsa_miR_200a (increased), hsa_miR_518a (increased), hsa_miR_523 (decreased), hsa_miR_520a_AS (decreased), hsa_miR_512_5p (decreased), hsa_miR_30d (increased), hsa_miR_138 (increased), hsa_miR_200c* (increased), hsa_miR_200b (increased), hsa_miR_296 (decreased), hsa_miR_340* (decreased), hsa_miR_517 (increased), hsa_miR_181c (increased), hsa_miR_27a (decreased), hsa_miR_520b (decreased), hsa_miR_504 (decreased), hsa_miR_99a (decreased), hsa_miR_149 (decreased), hsa_miR_509 (decreased), hsa_miR_182 (increased), hsa_miR_147 (increased), hsa_miR_29a (increased), hsa_miR_151 (increased), hsa_miR_98 (increased), hsa_miR_215 (increased). In some embodiments, the reference miRNA expression profile is that recited parenthetically after the aforementioned miRNAs (increased or decreased) and is indicative of the reference cell's resistance to the anti-cancer agent in question.

### BRIEF DESCRIPTION OF THE DRAWINGS

For a fuller understanding of the invention, reference should be made to the following detailed description, taken in connection with the accompanying drawings, in which:

FIG. 1 is a graph showing modulation of miR-367 expression. Renal cell lines, 786-0 and TK-10 were transfected with the precursor (gray, Pre-miR-367) and the inhibitor (black, Anti-miR-367) to miRNA 367 and evaluated for expression by QPCR. A non-targeting miRNA inhibitor served as the control (white) and expression was normalized to endogenous RNU44.

FIGS. 2A and 2B are graphs showing modulation of miR-367 expression changes topotecan sensitivity in 786-0 cells (FIG. 2A) and TK-10 cells (FIG. 2B). Renal cancer cells, 786-0 and TK-10 were incubated with increasing concentrations of topotecan, 24 hours after transfection with the precursor (circles) and inhibitor (triangles) to miR-367. Cell viability was assessed at 48 hours by CellTiter-Glo™ luminescent cell viability assay kit. There was a significant decrease in topotecan-induced cell death and growth arrest in 786-0 cells (p<0.05, topotecan sensitive, high endogenous miR-367) transfected with the miRNA inhibitor, as shown in FIG. 2A. In contrast, FIG. 2B shows that there was a significant increase in topotecan-induced cell death and growth arrest in TK-10 cells (p<0.02, topotecan resistant, low endogenous miR-367) transfected with the precursor to miR-367. A non-targeting miRNA served as the control (squares).

FIG. 3 is a table listing cell lines subject to miRNA expression analysis and their tissue of origin.

FIGS. 4A-G are tables of microRNAs associated with in vitro sensitivity/resistance (GI₅₀) to cytotoxic agents in 40 human cancer cell lines.

FIGS. 5A and 5B show a sigmoidal topotecan dose-response curve and fold changes, respectively, in ChicisR cells following transfection with miR302b precursor.

FIGS. 5C and 5D show a sigmoidal topotecan dose-response curve and fold changes, respectively in ChicisR cells following transfection with anti-miR302b inhibitor.

FIGS. 6A and 6B show ChicisR cell viability following transfection with miR302 precursor (FIG. 6A) or anti-miR302b (FIG. 6B), and treatment with topotecan.

FIGS. 7A and 7B show a sigmoidal paclitaxel dose-response curve and fold changes, respectively, in ChicisR cells following transfection with miR30a5p precursor.

FIGS. 7C and 7D show a sigmoidal paclitaxel dose-response curve and fold changes, respectively, in ChicisR cells following transfection with anti-miR30a5p inhibitor.

FIGS. 8A and 8B show ChicisR cell viability following transfection with miR30a5p precursor (FIG. 8A) or anti-miR30a5p inhibitor (FIG. 8B), and treatment with paclitaxel.

FIGS. 9A and 9B show a sigmoidal topotecan dose-response curve and fold changes, respectively, in ChicisR cells following transfection with miR30a5p precursor.

FIGS. 9C and 9D show a sigmoidal topotecan dose-response curve and fold changes, respectively, in ChicisR cells following transfection with anti-miR30a5p inhibitor.

FIGS. 10A and 10B show ChicisR cell viability following transfection with miR30a5p precursor (FIG. 10A) or anti-miR30a5p inhibitor (FIG. 10B), and treatment with topotecan.

FIGS. 11A and 11B show a sigmoidal paclitaxel dose-response curve and fold changes, respectively, in OVCAR-4 cells following transfection with miR302b precursor.

FIGS. 11C and 11D show a sigmoidal paclitaxel dose-response curve and fold changes, respectively, in OVCAR-4 cells following transfection with anti-miR302b inhibitor.

FIGS. 12A and 12B show OVCAR-4 cell viability following transfection with miR302b precursor (FIG. 12A) or anti-miR302b inhibitor (FIG. 12B), and treatment with paclitaxel.

FIGS. 13A and 13B show a sigmoidal topotecan dose-response curve and fold changes, respectively, in OVCAR-4 cells following transfection with miR302b precursor.

FIGS. 13C and 13D show a sigmoidal topotecan dose-response curve and fold changes, respectively, in OVCAR-4 cells following transfection with anti-miR302b inhibitor.

FIGS. 14A and 14B show OVCAR-4 cell viability following transfection with miR30a5p precursor (FIG. 14A) or anti-miR30a5p inhibitor (FIG. 14B), and treatment with topotecan.

FIGS. 15A and 15B show a sigmoidal paclitaxel dose-response curve and fold changes, respectively, in OVCAR-4 cells following transfection with miR30a5p precursor.

FIGS. 15C and 15D show a sigmoidal paclitaxel dose-response curve and fold changes, respectively, in OVCAR-4 cells following transfection with anti-miR30a5p inhibitor.

FIGS. 16A and 16B show OVCAR-4 cell viability following transfection with miR30a5p precursor (FIG. 16A) or anti-miR30a5p inhibitor (FIG. 16B), and treatment with paclitaxel.

FIGS. 17A and 17B show a sigmoidal paclitaxel dose-response curve and fold changes, respectively, in SKOV-4 cells following transfection with miR30a5p precursor.

FIGS. 17C and 17D show a sigmoidal paclitaxel dose-response curve and fold changes, respectively, in SKOV-4 cells following transfection with anti-miR30a5p inhibitor.

FIGS. 18A and 18B show SKOV-4 cell viability following transfection with miR30a5p precursor (FIG. 18A) or anti-miR30a5p inhibitor (FIG. 18B), and treatment with paclitaxel.

FIGS. 19A and 19B show a sigmoidal paclitaxel dose-response curve and fold changes, respectively, in PA1 cells following transfection with anti-miR367 inhibitor.

FIG. 20 shows PA1 cell viability following transfection with miR367 inhibitor and treatment with paclitaxel.

FIGS. 21A and 21B show a sigmoidal topotecan dose-response curve and fold changes, respectively, in MCF-7 cells following transfection with miR30a5p precursor.

FIGS. 21C and 21D show a sigmoidal topotecan dose-response curve and fold changes, respectively, in MCF-7 cells following transfection with anti-miR30a5p inhibitor.

FIGS. 22A and 22B show MCF-7 cell viability following transfection with miR30a5p precursor (FIG. 22A) or anti-miR30a5p inhibitor (FIG. 22B), and treatment with topotecan.

FIGS. 23A and 23B show a sigmoidal paclitaxel dose-response curve and fold changes, respectively, in Hs578T cells following transfection with miR367 precursor.

FIGS. 23C and 23D show a sigmoidal paclitaxel dose-response curve and fold changes, respectively, in Hs578T cells following transfection with anti-miR367 inhibitor.

FIGS. 24A and 24B show Hs578T cell viability following transfection with miR367 precursor (FIG. 24A) or anti-miR367 inhibitor (FIG. 24B), and treatment with paclitaxel.

FIGS. 25A and 25B show a sigmoidal topotecan dose-response curve and fold changes, respectively, in Hs578T cells following transfection with miR367 precursor.

FIGS. 25C and 25D show a sigmoidal topotecan dose-response curve and fold changes, respectively, in Hs578T cells following transfection with anti-miR367 inhibitor.

FIGS. 26A and 26B show Hs578T cell viability following transfection with miR367 precursor (FIG. 26A) or anti-miR367 inhibitor (FIG. 26B), and treatment with topotecan.

FIGS. 27A and 27B show a sigmoidal paclitaxel dose-response curve and fold changes, respectively, in Hs578T cells following transfection with miR30a5p precursor.

FIGS. 27C and 27D show a sigmoidal paclitaxel dose-response curve and fold changes, respectively, in Hs578T cells following transfection with anti-miR30a5p inhibitor.

FIGS. 28A and 28B show Hs578T cell viability following transfection with miR30a5p precursor (FIG. 28A) or anti-miR30a5p inhibitor (FIG. 28B), and treatment with paclitaxel.

FIGS. 29A and 29B show a sigmoidal topotecan dose-response curve and fold changes, respectively, in Hs578T cells following transfection with miR30a5p precursor.

FIGS. 29C and 29D show a sigmoidal topotecan dose-response curve and fold changes, respectively, in Hs578T cells following transfection with anti-miR30a5p inhibitor.

FIGS. 30A and 30B show Hs578T cell viability following transfection with miR30a5p precursor (FIG. 30A) or anti-miR30a5p inhibitor (FIG. 30B), and treatment with topotecan.

### BRIEF DESCRIPTION OF THE SEQUENCES

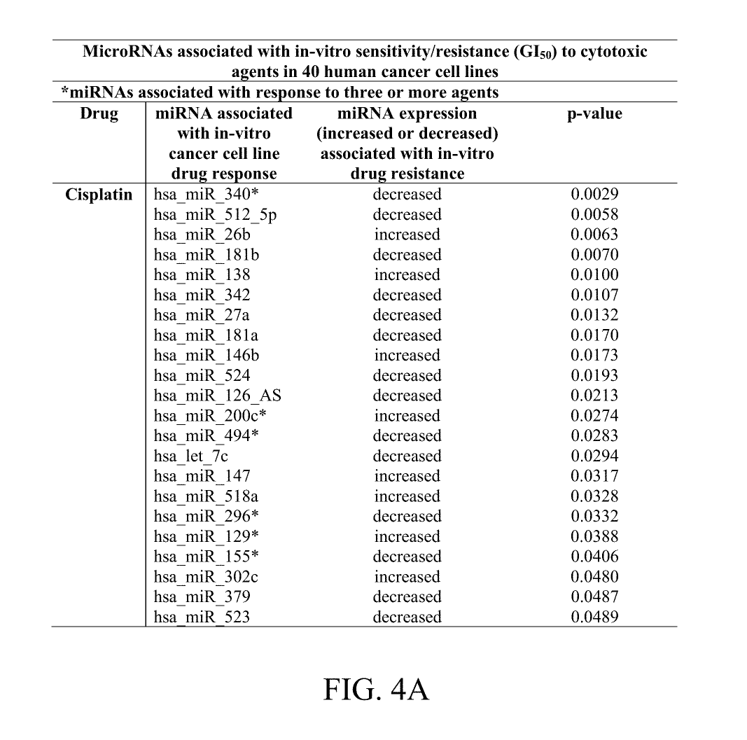

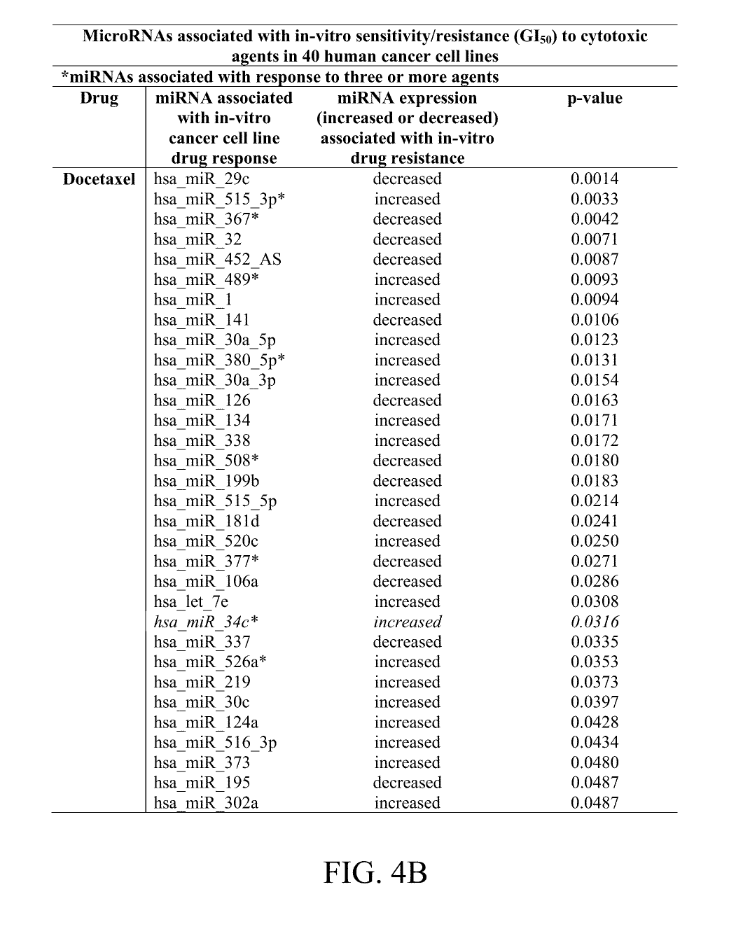

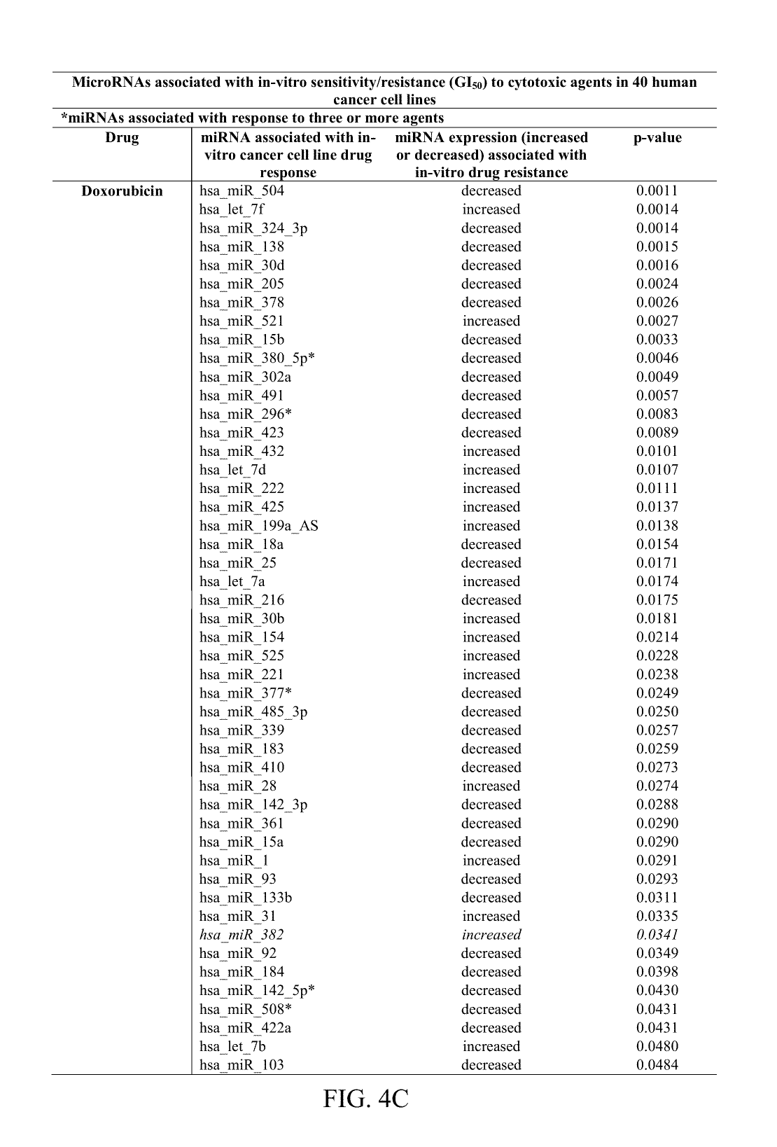

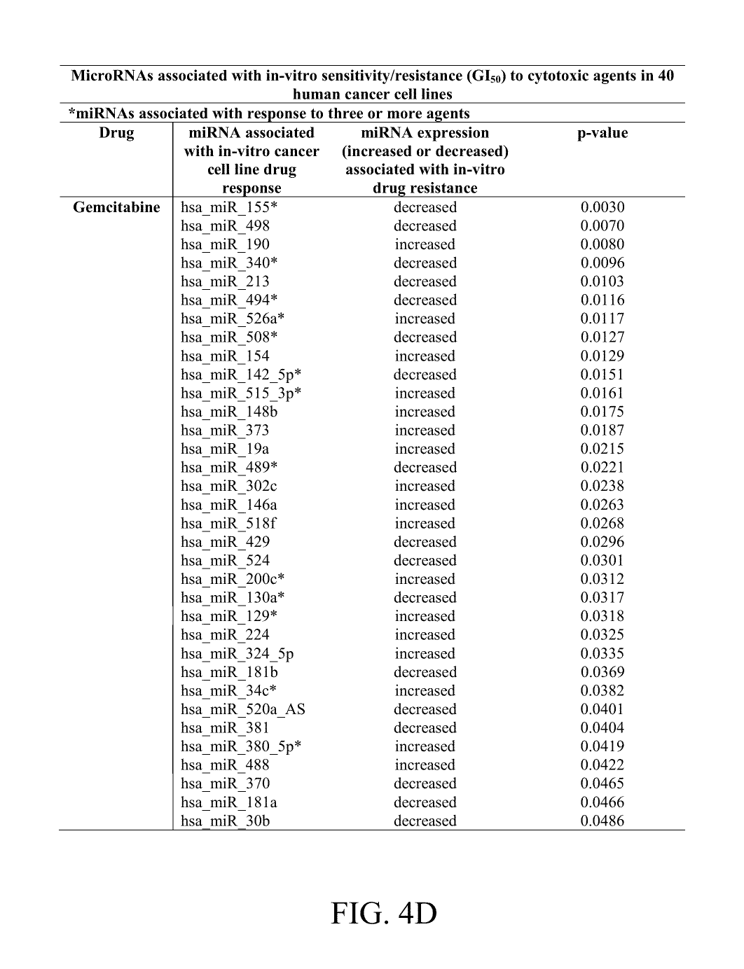

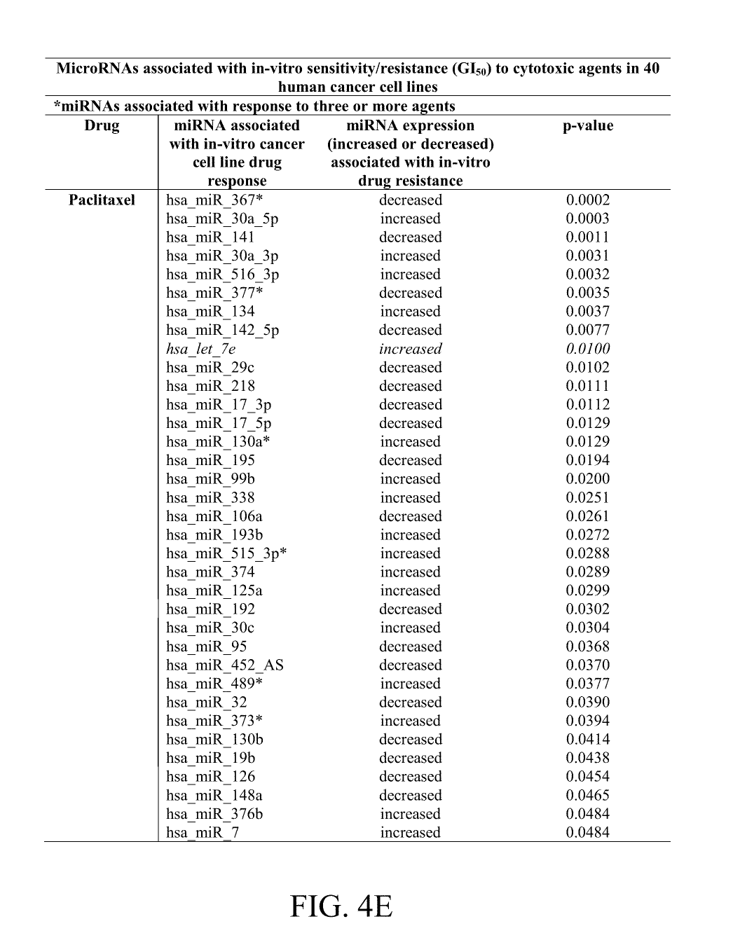

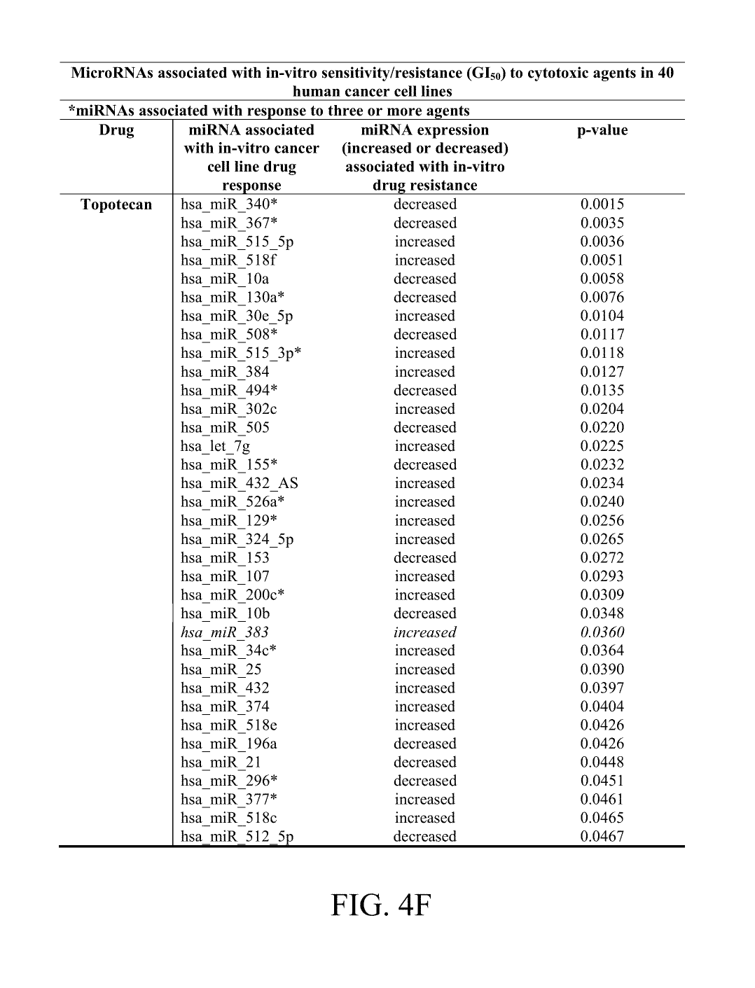

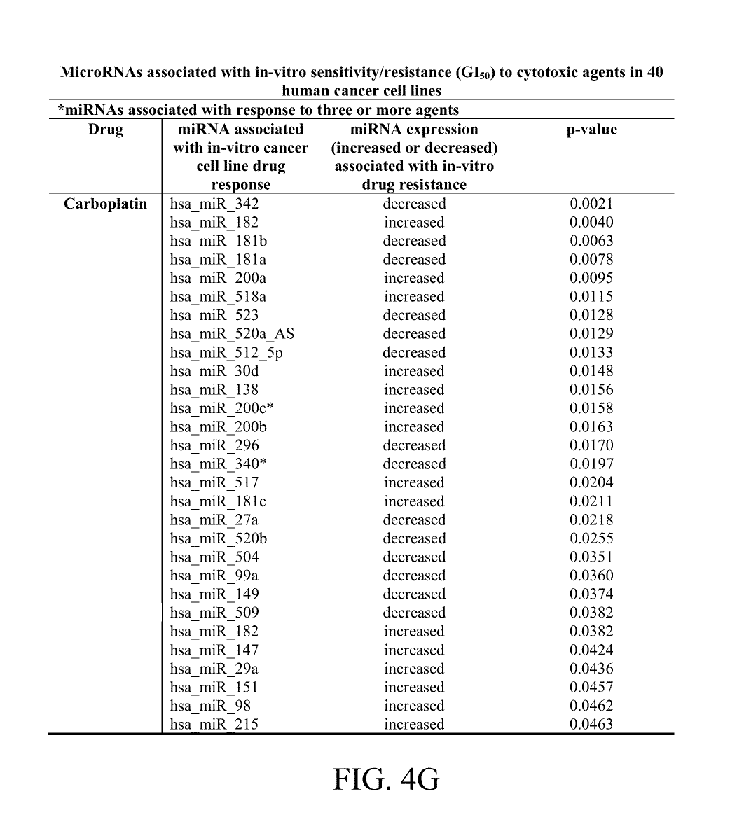

SEQ ID NOs: 1-157 are human miRNAs associated with in vitro cancer cell line drug resistance (FIGS. 4A-4G; Table 1).

SEQ ID NO: 158 is the sequence of an exemplified pre-miR367 precursor: ccauuacuguugcuaauaugcaacucuguugaauauaaauuggaauugcacuuuagcaauggugaugg (SEQ ID NO:158).

SEQ ID NO: 159 is the sequence of an exemplified pre-miR302b precursor: gcucccuucaacuuuaacauggaagugcuuucugugacuuuaaaaguaagugcuuccauguuuuaguaggagu (SEQ ID NO:159).

SEQ ID NO: 160 is the sequence of an exemplified pre-miR30a (miR30a-5p) precursor: gcgacuguaaacauccucgacuggaagcugugaagccacagaugggcuuucagucggauguuugcagcugc (SEQ ID NO:160).

### DETAILED DESCRIPTION OF THE INVENTION

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention

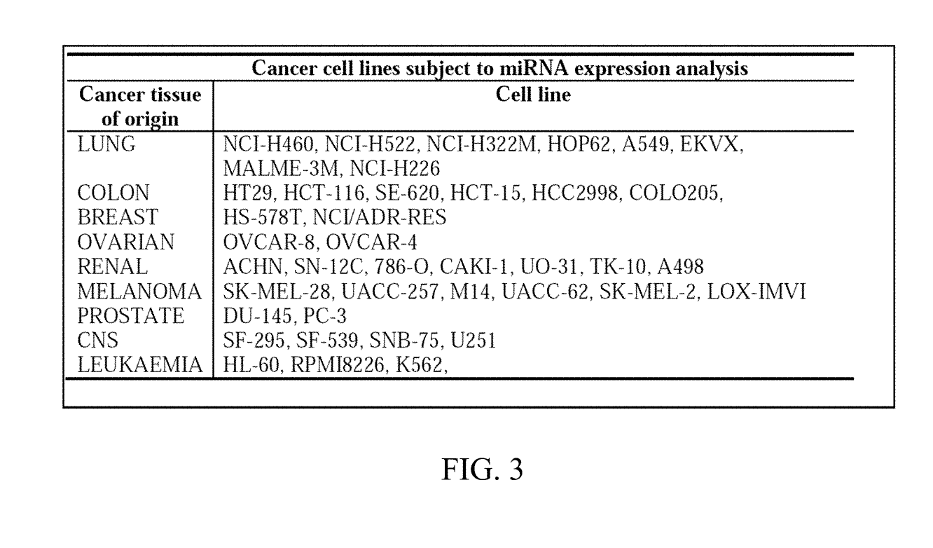

The inventors sought to evaluate how global miRNA expression levels influence chemosensitivity in a series of human cancer cell types (FIG. 3). The inventors have taken advantage of a subset (n=40) of the NCI 60 panel of cells, which has been used by the Developmental Therapeutics Program (DTP) of the U.S. National Cancer Institute (NCI) to screen more than 100,000 chemical compounds, and for which chemosensitivity data is available for many cytotoxic agents currently used in gynecologic oncology practice [16].

The inventors have integrated miRNA data for lung, colon, breast, ovary, kidney, skin (melanoma), prostate, central nervous system (CNS), and hematologic (leukemia) cancer cell lines with GI50 chemo-sensitivity data for doxorubicin, paclitaxel, topotecan, gemcitabine, docetaxel, cisplatin and carboplatin in an effort to identify miRNAs associated with chemo-response that may be explored as therapeutic targets.

Embodiments of the methods of the invention include predicting or determining the sensitivity of cancer cells to an anti-cancer agent by determining relative levels (elevated or decreased) of selected miRNAs in a sample of the cancer cells, such as a sample obtained from a human or non-human mammal, compared to a reference level. The sample expression levels and reference expression levels may be expressed by any method useful for comparison purposes, such as a numeric value, score, cutoff (threshold), or other expression. The method of sampling is not intended to be a limiting factor and is at the discretion of the care giver. Samples may be obtained and/or analyzed from subjects known to have cancer or suspected of having cancer, for example. Preferably, the reference expression profile is that of a reference cancer cell line that is appropriately matched to the cancer or suspected cancer in the subject. For example, in cases in which the sample comprises lung cancer cells, an expression profile of one or more lung cancer cell lines would typically be selected for comparison to assess differences in miRNA expression.

The term “miRNA” is used according to its ordinary and plain meaning and refers to a microRNA molecule found in eukaryotes that is involved in RNA-based gene regulation. See, e.g., Carrington et al., Science, 2003, 301(5631):336-338, which is hereby incorporated by reference. Individual miRNAs in a variety of organisms have been identified, sequenced, and given names. Names of miRNAs and their sequences related to the present invention are provided herein. As used herein, “hsa” in the name of an miRNA (for example, hsa_miR_340) refers to the human miRNA sequence. In some embodiments of each of the methods and compositions disclosed herein, the miRNA sequence that can be used in the context of the invention include, but are not limited to, one or more of those miRNA sequences in FIGS. 4A-4G, Table 1, and SEQ ID NOs: 1-157.

It is understood that a “synthetic nucleic acid” of the invention means that the nucleic acid does not have a chemical structure or sequence of a naturally occurring nucleic acid or is made by non-natural processes. Consequently, it will be understood that the term “synthetic miRNA” refers to a “synthetic nucleic acid” that functions as or inhibits the functions of an miRNA, at least in part, in a cell or under physiological conditions.

While some of the embodiments of the invention involve synthetic miRNAs or synthetic nucleic acids, in some embodiments of the invention, the nucleic acid molecule(s) need not be “synthetic.” In certain embodiments, a non-synthetic miRNA employed in methods and compositions of the invention may have all or part of the sequence and structure of a naturally occurring miRNA precursor or the mature miRNA. For example, non-synthetic miRNAs used in methods and compositions of the invention may not have one or more modified nucleotides or nucleotide analogs. In these embodiments, the non-synthetic miRNA may or may not be recombinantly produced. In particular embodiments, the nucleic acid in methods and/or compositions of the invention is specifically a synthetic miRNA; though in other embodiments, the invention specifically involves a non-synthetic miRNA and not a synthetic miRNA. Any embodiments discussed with respect to the use of synthetic miRNAs can be applied with respect to non-synthetic miRNAs, and vice versa. The synthetic miRNAs and non-synthetic miRNAs may be in isolated form.

It will be understood that the term “naturally occurring” refers to something found in an organism without any intervention by a person; it could refer to a naturally-occurring wild-type or mutant molecule. In some embodiments a synthetic miRNA molecule does not have the sequence of a naturally occurring miRNA molecule. In other embodiments, a synthetic miRNA molecule may have the sequence of a naturally occurring miRNA molecule, but the chemical structure of the molecule, particularly in the part unrelated specifically to the precise sequence (non-sequence chemical structure) differs from chemical structure of the naturally occurring miRNA molecule with that sequence. In some cases, the synthetic miRNA has both a sequence and non-sequence chemical structure that are not found in a naturally-occurring miRNA. Moreover, the sequence of the synthetic molecules will identify which miRNA is effectively being provided or inhibited; the endogenous miRNA will be referred to as the “corresponding miRNA” or “target miRNA”. Corresponding miRNA sequences that can be used in the context of the invention include, but are not limited to, one or more of those sequences in FIGS. 4A-4G, Table 1, and SEQ ID NOs: 1-157, as well as any other of miRNA sequence, miRNA precursor sequence, or any complementary sequence. In some embodiments, the sequence is or is derived from or contains all or part of a sequence identified in Table 1 to target a particular miRNA (or set of miRNAs). In some embodiments, the miRNA precursor sequence comprises SEQ ID NO:158, SEQ ID NO:159, or SEQ ID NO:160.

In some embodiments, it may be useful to know whether a cell expresses a particular miRNA endogenously or whether such expression is affected under particular conditions or a particular disease state. Thus, in some embodiments of the invention, methods include assaying a cell or a sample containing a cell for the presence of one or more miRNA. In other aspects, a sample may comprise RNA or nucleic acid isolated from a tissue or cells of a subject or reference cells (for example, reference cells from a cancer cell line known to be sensitive to an anti-cancer agent in question or known to be resistant to an anti-cancer agent in question). Consequently, in some embodiments, methods include a step of generating an miRNA profile for a sample. The term “miRNA profile” refers to a set of data regarding the expression pattern for one or more miRNAs (e.g., one or a plurality of miRNA from SEQ ID NOs:1-157, Table 1, FIG. 4A, FIG. 4B, FIG. 4C, FIG. 4D, FIG. 4E, FIG. 4F, or FIG. 4G) in the sample; it is contemplated that the miRNA profile can be obtained using a set of miRNAs, using for example nucleic acid amplification or hybridization techniques well known to one of ordinary skill in the art. It is contemplated that any one or subset of the miRNA listed in this application can be included or excluded from the claimed invention.

In some embodiments of the invention, an miRNA profile is generated by steps that include: (a) labeling miRNA in the sample; (b) hybridizing miRNA to a number of probes, or amplifying a number of miRNA, and (c) determining miRNA hybridization to the probes or detection of miRNA amplification products, wherein an miRNA profile is generated.

Methods of the invention involve predicting the response of a cancer in a mammalian subject (such as a human patient) to an anti-cancer agent based on an miRNA expression or expression profile of a sample of the cancer obtained from the subject. In certain embodiments, the presence, absence, elevation, or reduction in the level of expression of a particular miRNA or set of miRNA in a cell is correlated with a state of resistance or sensitivity to an anti-cancer agent of interest compared to a reference expression level, such as the expression level of that miRNA or set of miRNAs in a cancer cell line of the same cancer time sampled from the subject, wherein the cancer cell line is pre-determined or known to be resistant or sensitive to the anti-cancer agent in question (or to be resistant or sensitive to a variant of the anti-cancer agent in question). This correlation allows for prognostic methods and treatment selection to be carried out when the expression level of an miRNA is measured in a biological sample being assessed and then compared to the expression level of a reference cell (reference expression profile). It is specifically contemplated that miRNA profiles for subjects, particularly those suspected of having a cancer, can be generated by evaluating any miRNA or sets of the miRNAs disclosed in this application. The miRNA profile that is generated from the subject will be one that provides information regarding the cancer. In many embodiments, the miRNA profile is generated using miRNA hybridization or amplification, (e.g., array hybridization or RT-PCR). In certain aspects, a miRNA profile can be used in conjunction with other diagnostic and prognostic tests, such as serum protein profiles.

Embodiments of the invention include obtaining an miRNA expression profile of one or more miRNAs in a sample from a subject. The difference in the miRNA expression profile in the sample from the subject and a reference miRNA expression profile is indicative of a state of sensitivity or a state of resistance to an anti-cancer agent in question (such as a cytotoxic agent) or class of anti-cancer agent. The sample miRNA expression profile and/or the reference miRNA expression profile can be expressed in any format or readout amenable to comparison, such as a digital reference, for example. An miRNA probe array or probe set comprising or identifying a segment of a corresponding miRNA can include all or part of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 125, 150, to 157, or or any integer or range derivable there between, of a miRNA or its complement listed in Table 1, SEQ ID NOs:1-157, FIG. 4A, FIG. 4B, FIG. 4C, FIG. 4D, FIG. 4E, FIG. 4F, or FIG. 4G. It is contemplated the any one or subset of the miRNA listed in this application can be included or excluded from the claimed invention.

A sample may be taken from a subject having or suspected of having cancer. A sample may also comprise nucleic acids or RNA isolated from a tissue or cell sample from a subject. In certain aspects, the sample can be, but is not limited to tissue (e.g., biopsy, particularly fine needle biopsy, excision, or punch biopsy), blood, serum, plasma. The sample can be fresh, frozen, fixed (e.g., formalin fixed), or embedded (e.g., paraffin embedded) tissues or cells. In a particular aspect, the sample is a sample of lung cancer cells, colon cancer cells, breast cancer cells, ovarian cancer cells, renal cancer cells, melanoma cells, prostate cancer cells, CNS cancer cells, or leukemia cells, or nucleic acid or RNA isolated therefrom.

The methods can further comprise one or more steps including: (a) obtaining a sample from the subject, (b) isolating nucleic acids from the sample, (c) labeling the nucleic acids isolated from the sample, and (d) hybridizing the labeled nucleic acids to one or more probes or primers. Nucleic acids of the invention include one or more nucleic acid comprising at least one segment having a sequence or complementary sequence of one or more of the miRNA sequences disclosed herein (e.g., SEQ ID NOs:1-160, Table 1, FIGS. 4A-4G). Nucleic acids of the invention are typically coupled to a support. Such supports are well known to those of ordinary skill in the art and include, but are not limited to glass, plastic, metal, or latex. In particular aspects of the invention, the support can be planar or in the form of a bead or other geometric shapes or configurations.

Certain embodiments of the invention include determining expression of one or more miRNA by using an amplification assay or a hybridization assay, a variety of which are well known to one of ordinary skill in the art. In certain aspects, an amplification assay can be a quantitative amplification assay, such as quantitative RT-PCR or the like. In still further aspects, a hybridization assay can include in situ hybridization, array hybridization assays or solution hybridization assays.

Embodiments of the invention concern agents (e.g., nucleic acids) that increase the level of, or perform the activities of, endogenous miRNA, when introduced into cells in vitro or in vivo for example, precursor microRNA (pre-miRNA). Embodiments of the invention also include agents (e.g., nucleic acids) that decrease the level of or inhibit endogenous miRNAs when introduced into cells in vitro or in vivo, for example, anti-microRNA inhibitors (anti-mRNA). In certain aspects, nucleic acids are synthetic or non-synthetic miRNA. Sequence-specific anti-miRNA inhibitors can be used to inhibit sequentially or in combination the activities of one or more endogenous miRNAs in cells, as well those genes and associated pathways modulated by the endogenous miRNA.

In some embodiments, the agent that inhibits or decreases the level of one or more miRNAs comprises one or more among an anti-miRNA oligonucleotide (AMO), multiple-target AMO (MT-AMO), miRNA sponge, miRNA masking antisense oligonucleotide, and miRNA knockout agent. In some embodiments, the agent that inhibits or decreases the level of one or more miRNA comprises an antisense oligonucleotide (ASO) having a backbone modification or 2′ sugar modification selected from 2′-O-methyl (2′-O-Me), 2′-O-methoxyethyl (2′-MOE), 2′-fluoro (2′F), or locked nucleic acid (LNA). In some embodiments, the agent that inhibits or decreases the level of one or more miRNAs comprises an antagomir (anti-mRNA oligonucleotide (AMO) conjugated with cholesterol). Examples of agents that inhibit or decrease the level of miRNAs, which may be utilized in the invention, include but are not limited to, Blenkiron, C. et al. (2007) Human Molecular Genetics, 16(1):R106-R113; Davis, S. et al. (2009) Nucleic Acids Research, 37(1):70-77; Wang, Z. (2009) MicroRNA Interference Technologies, New York: Springer-Verlag Berlin Heidelberg, pp. 59-73; Cheng, A. et al. (2005) Nucleic Acids Research, 33(4):1290-1297; Horwich, M. et al. “Design and Delivery of Antisense Oligonucleotides to Block microRNA Function in Cultured Drosophilia and Human Cells” Nat Protoc, author manuscript available in PMC 2008 Oct. 3, pp. 1-29; Oh, S. et al. “A Highly Effective and Long-Lasting Inhibition of miRNAs with PNA-Based Antisense Oligonucleotides” Molecules and Cells, published online Sep. 30, 2009, pp. 1-5; Wang, P. et al. (2010) Mol Cell Biochem, 339(1-2):163-171, abstract; Lan, F. et al. (2008) J Drug Target, 16(9):688-693, abstract; Esau, C. (2008) Methods, 44(1):55-60, abstract; Stenvang, J. et al. (2008) Expert Opin Biol Ther, 8(1):59-81, abstract; Ørom, U. et al. (2006) Gene, 372:137-141, abstract; Stenvang, J. et al. (2008) Semin Cancer Biol, 18(2):89-102, abstract; Park, J-K et al. (2009) Pancreas, 38(7):e190-e199, abstract; Galluzzi, L. et al. (2010) Cancer Res, 70:1793-1803; and Esquela-Kerscher, A. et al. (2006) Nature Reviews, 6:259-269, which are each incorporated herein by reference.

In some embodiments, the agents that increase the level of, or perform the activities of, endogenous miRNA, and the agents that inhibit or decrease the level of one or more endogenous miRNAs are short nucleic acid molecules. The term “short” refers to a length of a single polynucleotide that is 5, 10, 15, 20, 25, 50, 100, or 150 nucleotides or fewer, including all integers or range derivable there between.

The present invention also concerns kits containing compositions of the invention or compositions to implement methods of the invention. In some embodiments, kits can be used to evaluate one or more miRNA molecules. In certain embodiments, a kit contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 300, 400, 500 or more miRNA probes, miRNA molecules or miRNA inhibitors, or any range and combination derivable therein. In some embodiments, there are kits for evaluating or modulating miRNA activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using miRNA probes or primers, synthetic miRNAs, nonsynthetic miRNAs, and/or miRNA inhibitors of the invention for therapeutic or prognostic applications are also included as part of the invention. Specifically contemplated are any such molecules corresponding to any miRNA reported to influence biological activity, such as those discussed herein.

In certain aspects, negative and/or positive control pre-miRNAs and/or anti-miRNA inhibitors are included in some kit embodiments. The control molecules can be used to verify transfection efficiency and/or control for transfection-induced changes in cells.

It is contemplated that any method or composition described herein can be implemented with respect to any other method or composition described herein and that different embodiments may be combined. It is specifically contemplated that any methods and compositions discussed herein with respect to miRNA molecules or miRNA may be implemented with respect to synthetic miRNAs to the extent the synthetic miRNA is exposed to the proper conditions to allow it to become a mature miRNA under physiological circumstances. The claims originally filed are contemplated to cover claims that are multiply dependent on any filed claim or combination of filed claims.

It is also contemplated that any one or more of the miRNA listed, particularly in Table 1, may be specifically excluded from any particular set or subset of miRNA or nucleic acid.

Any embodiment of the invention involving specific miRNAs by name is contemplated also to cover embodiments involving miRNAs whose sequences are at least 70, 75, 80, 81, 82, 83, 84, 85, 86, 87, 88, 89, 90, 91, 92, 93, 94, 95, 96, 97, 98% identical to the mature sequence of the specified miRNA. This also includes the various fragments of these miRNA or nucleic acid sequences.

Embodiments of the invention include kits for analysis of a pathological sample by assessing miRNA expression profile for a sample comprising, in suitable container(s), two or more miRNA probes, wherein the miRNA probes detect one or more of the miRNAs described herein (e.g., FIGS. 4A-4G, Table 1, SEQ ID NOs:1-160. The kit can further comprise reagents for labeling miRNA in the sample. The kit may also include labeling reagents, for example, at least one amine-modified nucleotide, poly(A) polymerase, and poly(A) polymerase buffer. Labeling reagents can include an amine-reactive dye.

It will be understood that shorthand notations are employed such that a generic description of an miRNA refers to any of its gene family members (distinguished by a number or sequence similarity), unless otherwise indicated. It is understood by those of skill in the art that a “gene family” refers to a group of genes having the same or similar miRNA coding sequence. Typically, members of a gene family are identified by a number following the initial designation; however, some family members are identified by sequence similarity, for example see the various miRNA databases. For example, miR-16-1 and miR-16-2 are members of the miR-16 gene family and “mir-7” refers to miR-7-1, miR-7-2 and miR-7-3. Moreover, unless otherwise indicated, a shorthand notation refers to related miRNAs (distinguished by a letter). Thus, “let-7” for example, refers to let-7a-1, let?-a-2, let-7b, let-7c, let-7d, let-7e, let-7f-1, and let-7f-2. Exceptions to this shorthand notations will be otherwise identified.

The terms “introducing”, “administering”, “providing”, and “contacting” are used interchangeably herein to refer to delivering the substance in question (e.g., an anti-cancer agent or an agent that increases or decreases that activity or level of an miRNA) to a human or non-human mammalian cell in vitro or in vivo (e.g., to a human or non-human mammalian subject).

The terms “inhibiting,” “reducing,” or “prevention,” or any variation of these terms, when used in the claims and/or the specification includes any measurable decrease or complete inhibition to achieve a desired result.

The use of the word “a” or “an” may mean “one,” but it is also consistent with the meaning of “one or more,” “at least one,” and “one or more than one.” For example, a cancer cell sample comprising “a cancer cell” means one or a plurality of cancer cells, and administration of an agent to a cell means administration of the agent to one or a plurality of cells. An anti-cancer agent is inclusive of a single anti-cancer agent (e.g., monotherapy) and a plurality of anti-cancer agents (e.g., combination therapy). Thus, predicting the responsiveness (sensitivity/resistance) to combinations of anti-cancer agents is contemplated.

The terms “subject”, “patient”, and “individual” are used herein interchangeably to refer to human and non-human mammals. Unless specified by context (e.g., type of disease a subject is afflicted with), the subject may be any age and gender.

The term “anti-cancer agent” is used herein to refer to agents (e.g., small molecules and biologic molecules) effective for the treatment of cancers. In some embodiments, the anti-cancer agent is a chemotherapeutic drug, which typically act by killing cells that divide rapidly, one of the main properties of most cancer cells. Examples of chemotherapeutic drugs include but are not limited to alkylating agent, antimetabolite, anthracycline, plant alkaloid, topoisomerase inhibitor, or other anti-tumor agent. In some embodiments, the anti-cancer agent comprises one or more cytotoxic agents from among doxorubicin, paclitaxel, topotecan, gemcitabine, docetaxel, cisplatin and caraboplatin, or a variant of any of the foregoing. Variant cytotoxic agents are known in the art. For example, cisplatin variant include but are not limited to carboplatin, tetraplatin, oxaliplatin, aroplatin, and transplatin. In some embodiments, the anti-cancer agent comprises one or more from among a platinum compound, DNA repair inhibitor, angiogenesis inhibitor, or PI3 kinase inhibitor.

The terms “predict,” “predicting” and “prediction” as used herein does not necessarily mean that the event will happen with 100% certainty; rather, it is intended to mean the event will more likely occur than not occur. The miRNAs and miRNA expression profiles, and methods and compositions disclosed herein may be used to predict the response of a cancer to an anti-cancer agent (or combination of anti-cancer agents) or class of anti-cancer agents in vitro and/or in vivo based on the compared resistance/sensitivity exhibited by a reference miRNA expression profile, such as that obtained from a cancer cell line for which the responsiveness of the cancer cell line to the anti-cancer agent (or combination of anti-cancer agents) or class of anti-cancer agent in question is known. Based on the predicted responsiveness, an anti-cancer agent or combination of anti-cancer agents can be selected that is most likely to be effective in treating the subject and/or reduce the amount of anti-cancer agent that must be administered to the agent to obtain a clinical benefit. Thus, the miRNAs, miRNA expression profiles, and methods and compositions disclosed herein may be used to increase the safety and effectiveness of cancer treatment. Optionally, once the responsiveness of a cancer in a subject to an anti-cancer agent is predicted based on comparison of miRNA expression profile to a reference miRNA expression profile, the prediction can be verified in vitro prior to administration of the anti-cancer agent (or combination of anti-cancer agents) to the subject by treating cancer cells from the subject in vitro with the anti-cancer agent and observing the response.

The terms “treatment”, “treating” and the like are intended to mean obtaining a desired pharmacologic and/or physiologic effect, e.g., slowing or stopping cancer progression, time to relapse, or alleviating one or more symptoms of a disorder such as cancer. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease (for example, cancer) in a mammal, particularly a human, and includes: (a) preventing a disease or condition (e.g., preventing cancer) from occurring or recurring in an individual who may be predisposed to the disease but has not yet been diagnosed as having it; (b) inhibiting the disease, (e.g., arresting its development); or (c) relieving the disease (e.g., reducing symptoms associated with the disease). In some embodiments, the subject is suffering from the disorder (e.g., cancer), and treatment includes identifying the subject as suffering from the disorder (e.g., cancer) prior to administration of an effective amount of an agent such as anti-cancer agent or an agent that modulates a target miRNA (e.g., one or more among FIGS. 4A-4G, Table 1 and SEQ ID NOs:1-157).

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

miRNA Molecules

MicroRNA molecules (“miRNAs”) are generally 21 to 22 nucleotides in length, though lengths of 19 and up to 23 nucleotides have been reported. The miRNAs are each processed from a longer precursor RNA molecule (“precursor miRNA”). Precursor miRNAs are transcribed from non-protein-encoding genes. The processed miRNA (also referred to as “mature miRNA”) become part of a large complex to down-regulate a particular target gene.

The nucleic acid molecules of the invention may be synthetic. The term “synthetic” means the nucleic acid molecule is isolated and not identical in sequence and/or chemical structure to a naturally-occurring nucleic acid molecule, such as an endogenous precursor miRNA or miRNA molecule. While in some embodiments, nucleic acids of the invention do not have an entire sequence that is identical to a sequence of a naturally-occurring nucleic acid, such molecules may encompass all or part of a naturally-occurring sequence. It is contemplated, however, that a synthetic nucleic acid administered to a cell may subsequently be modified or altered in the cell such that its structure or sequence is the same or similar as non-synthetic or naturally occurring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA. The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many embodiments of the invention, a nucleic acid is isolated by virtue of it having been synthesized in vitro separate from endogenous nucleic acids in a cell. It will be understood, however, that isolated nucleic acids may be subsequently mixed or pooled together in a variety of combinations.

In certain aspects, synthetic miRNA of the invention are RNA or RNA analogs. miRNA inhibitors may be DNA or RNA, or analogs thereof. miRNA and miRNA inhibitors of the invention are typically “synthetic nucleic acids.”

In some embodiments, there is a recombinant or synthetic miRNA having a length of between 17 and 130 residues.

In certain embodiments, synthetic miRNA have (a) an “miRNA region” whose sequence from 5′ to 3′ is identical to all or a segment of a mature miRNA sequence, and (b) a “complementary region” whose sequence from 5′ to 3′ is between 60% and 100% complementary to the miRNA sequence. The term “miRNA region” refers to a region on the synthetic miRNA that is at least 75, 80, 85, 90, 95, or 100% identical, including all integers there between, to all or part of the sequence of a mature, naturally occurring miRNA sequence. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA.

The term “complementary region” refers to a region of a synthetic miRNA that is or is at least 60% complementary to a corresponding naturally occurring miRNA sequence. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary to its corresponding naturally occurring miRNA, or any range derivable therein. With single polynucleotide sequences, there can be a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active or functional strand.

In other embodiments of the invention, there are synthetic nucleic acids that are miRNA inhibitors, which may incorporate the designs of miRNA inhibitors disclosed in U.S. Patent Publication 2009/0186348 (Huibregtse S. B. et al., published Jul. 23, 2009), which is incorporated herein by reference. An miRNA inhibitor is typically between about 17 to 25 nucleotides in length and comprises a 5′ to 3′ sequence that is at least 90% complementary to the 5′ to 3′ sequence of a mature miRNA. In certain embodiments, an miRNA inhibitor molecule is 17, 18, 19, 20, 21, 22, 23, 24, or 25 nucleotides in length, or any range derivable therein. Moreover, an miRNA inhibitor has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. One of skill in the art could use a portion of a sequence that is complementary to the sequence of a mature miRNA as the sequence for an miRNA inhibitor. Moreover, that portion of a sequence can be altered so that it is still 90% complementary to the sequence of a mature miRNA.

In some embodiments of the invention, a synthetic miRNA contains one or more design elements. These design elements include, but are not limited to: (i) a replacement group for the phosphate or hydroxyl of the nucleotide at the 5′ terminus of the complementary region; (ii) one or more sugar modifications in the first or last 1 to 6 residues of the complementary region; or (iii) noncomplementarity between one or more nucleotides in the last 1 to 5 residues at the 3′ end of the complementary region and the corresponding nucleotides of the miRNA region.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an acetyl group, 2′O-Me (2′oxygen-methyl), DMTO (4,4′-dimethoxytrityl with oxygen), fluorescein, a thiol, or acridine, though other replacement groups are well known to those of skill in the art and can be used as well. This design element can also be used with an miRNA inhibitor.

Additional embodiments concern a synthetic miRNA having one or more sugar modifications in the first or last 1 to 6 residues of the complementary region (referred to as the “sugar replacement design”). In certain cases, there is one or more sugar modifications in the first 1, 2, 3, 4, 5, 6 or more residues of the complementary region, or any range derivable therein. In additional cases, there can be one or more sugar modifications in the last 1, 2, 3, 4, 5, 6 or more residues of the complementary region, or any range derivable therein. It will be understood that the terms “first” and “last” are with respect to the order of residues from the 5′ end to the 3′ end of the region. In particular embodiments, the sugar modification is a 2′O-Me modification. In further embodiments, there is one or more sugar modifications in the first or last 2 to 4 residues of the complementary region or the first or last 4 to 6 residues of the complementary region. This design element can also be used with an miRNA inhibitor. Thus, an miRNA inhibitor can have this design element and/or a replacement group on the nucleotide at the 5′ terminus, as discussed above.

In other embodiments of the invention, there is a synthetic miRNA in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that synthetic miRNA of the invention have one or more of the replacement, sugar modification, or noncomplementarity designs. In certain cases, synthetic RNA molecules have two of them, while in others these molecules have all three designs in place. The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides. When the RNA molecule is a single polynucleotide, there is a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length. In addition to having an miRNA region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

The present invention concerns miRNAs that can be labeled or amplified, used in array analysis, or employed in prognostic and therapeutic application. The RNA may have been endogenously produced by a cell, or been synthesized or produced chemically or recombinantly. They may be isolated and/or purified.

In some embodiments, a miRNA is used that does not correspond to a known human miRNA. It is contemplated that these non-human miRNA probes may be used in embodiments of the invention or that there may exist a human miRNA that is homologous to the non-human miRNA. While the invention is not limited to human miRNA, in certain embodiments, miRNA from human cells or a human biological sample is evaluated. In other embodiments, any mammalian cell, biological sample, or preparation thereof may be employed.

In some embodiments of the invention, methods and compositions involving miRNA may concern miRNA and/or other nucleic acids. Nucleic acids may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, miRNA containing vectors, control nucleic acids, and other probes and primers. In many embodiments, miRNA sequences are 19-24 nucleotides in length, while miRNA probes are 19-35 nucleotides in length, depending on the length of the processed miRNA and any flanking regions added. miRNA precursors are generally between 62 and 110 nucleotides in humans.

Nucleic acids, and mimetics thereof, of the invention may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous nucleotides. It is further understood that the length of complementarity within a precursor miRNA or between a miRNA probe and a miRNA or a miRNA gene are such lengths. Moreover, the complementarity may be expressed as a percentage, meaning that the complementarity between a nucleic acid and its target is 90% or greater over the length of the nucleic acid. In some embodiments, complementarity is or is at least 90%, 95% or 100%. In particular, such lengths may be applied to any nucleic acid comprising a nucleic acid sequence identified in any of SEQ ID NO:1 through SEQ ID NO:160, Table 1, FIGS. 4A-4G, or any other sequence disclosed herein. Each of these SEQ ID NOs is disclosed herein. The commonly used name of the miRNA is given (with its identifying source in the prefix, for example, “has” for human sequences) and the processed miRNA sequence. The invention include isolated miRNA probes. The term “miRNA probe” refers to a nucleic acid probe that can identify a particular miRNA or structurally related miRNAs, such as SEQ ID NO:1 through SEQ ID NO:160, those listed in Table 1, and those listed in FIGS. 4A-4G.

It is understood that a miRNA is derived from genomic sequences or a gene. In this respect, the term “gene” is used for simplicity to refer to the genomic sequence encoding the precursor miRNA for a given miRNA. However, embodiments of the invention may involve genomic sequences of a miRNA that are involved in its expression, such as a promoter or other regulatory sequences.

The term “recombinant” may be used and this generally refers to a molecule that has been manipulated in vitro or that is a replicated or expressed product of such a molecule.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.”

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary. miRNA probes or nucleic acids of the invention can include, can be or can be at least 60, 65, 70, 75, 80, 85, 90, 95, 96, 97, 98, 99 or 100% complementary to their target.

As used herein, “hybridization,” “hybridizes,” or “capable of hybridizing” is understood to mean the forming of a double or triple stranded molecule or a molecule with partial double or triple stranded nature. The term “anneal” as used herein is synonymous with “hybridize.” The term “hybridization,” “hybridize(s),” or “capable of hybridizing” encompasses hybridization under “stringent condition(s)” or “high stringency” and “low stringency” or “low stringency condition(s).”

As used herein “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42 degrees C. to about 70 degrees C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20 degrees C. to about 50 degrees C. Of course, it is within the skill of one in the art to further modify the low or high stringency conditions to suite a particular application.

A nucleic acid may comprise, or be composed entirely of, a derivative or analog of a nucleobase, a nucleobase linker moiety and/or backbone moiety that may be present in a naturally occurring nucleic acid. RNA with nucleic acid analogs may also be labeled according to methods of the invention. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described.

Additional non-limiting examples of nucleosides, nucleotides or nucleic acids comprising 5-carbon sugar and/or backbone moiety derivatives or analogs, include those in: U.S. Pat. No. 5,681,947, which describes oligonucleotides comprising purine derivatives that form triple helixes with and/or prevent expression of dsDNA; U.S. Pat. Nos. 5,652,099 and 5,763,167, which describe nucleic acids incorporating fluorescent analogs of nucleosides found in DNA or RNA, particularly for use as fluorescent nucleic acids probes; U.S. Pat. No. 5,614,617, which describes oligonucleotide analogs with substitutions on pyrimidine rings that possess enhanced nuclease stability; U.S. Pat. Nos. 5,670,663, 5,872,232 and 5,859,221, which describe oligonucleotide analogs with modified 5-carbon sugars (i.e., modified 2′-deoxyfuranosyl moieties) used in nucleic acid detection; U.S. Pat. No. 5,446,137, which describes oligonucleotides comprising at least one 5-carbon sugar moiety substituted at the 4′ position with a substituent other than hydrogen that can be used in hybridization assays; U.S. Pat. No. 5,886,165, which describes oligonucleotides with both deoxyribonucleotides with 3′-5′ internucleotide linkages and ribonucleotides with 2′-5′ internucleotide linkages; U.S. Pat. No. 5,714,606, which describes a modified internucleotide linkage wherein a 3′-position oxygen of the internucleotide linkage is replaced by a carbon to enhance the nuclease resistance of nucleic acids; U.S. Pat. No. 5,672,697, which describes oligonucleotides containing one or more 5′ methylene phosphonate internucleotide linkages that enhance nuclease resistance; U.S. Pat. Nos. 5,466,786 and 5,792,847, which describe the linkage of a substituent moiety which may comprise a drug or label to the 2′ carbon of an oligonucleotide to provide enhanced nuclease stability and ability to deliver drugs or detection moieties; U.S. Pat. No. 5,223,618, which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967, which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240, which describe oligonucleotides with three or four atom linker moiety replacing phosphodiester backbone moiety used for improved nuclease resistance, cellular uptake, and regulating RNA expression; U.S. Pat. No. 5,858,988, which describes hydrophobic carrier agent attached to the 2′-O position of oligonucleotides to enhanced their membrane permeability and stability; U.S. Pat. No. 5,214,136, which describes oligonucleotides conjugated to anthraquinone at the 5′ terminus that possess enhanced hybridization to DNA or RNA; enhanced stability to nucleases; U.S. Pat. No. 5,700,922, which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154, which describes RNA linked to a DNA to form a DNA-RNA hybrid; U.S. Pat. No. 5,728,525, which describes the labeling of nucleoside analogs with a universal fluorescent label.

Additional teachings for nucleoside analogs and nucleic acid analogs are U.S. Pat. No. 5,728,525, which describes nucleoside analogs that are end-labeled; U.S. Pat. Nos. 5,637,683, 6,251,666 (L-nucleotide substitutions), and U.S. Pat. No. 5,480,980 (7-deaza-2′deoxyguanosine nucleotides and nucleic acid analogs thereof).

Labeling methods and kits of the invention specifically contemplate the use of nucleotides that are both modified for attachment of a label and can be incorporated into a miRNA molecule. Such nucleotides include those that can be labeled with a dye, including a fluorescent dye, or with a molecule such as biotin. Labeled nucleotides are readily available; they can be acquired commercially or they can be synthesized by reactions known to those of skill in the art.

Modified nucleotides for use in the invention are not naturally occurring nucleotides, but instead, refer to prepared nucleotides that have a reactive moiety on them. Specific reactive functionalities of interest include: amino, sulfhydryl, sulfoxyl, aminosulfhydryl, azido, epoxide, isothiocyanate, isocyanate, anhydride, monochlorotriazine, dichlorotriazine, mono- or dihalogen substituted pyridine, mono- or disubstituted diazine, maleimide, epoxide, aziridine, sulfonyl halide, acid halide, alkyl halide, aryl halide, alkylsulfonate, N-hydroxysuccinimide ester, imido ester, hydrazine, azidonitrophenyl, azide, 3-(2-pyridyl dithio)-propionamide, glyoxal, aldehyde, iodoacetyl, cyanomethyl ester, p-nitrophenyl ester, o-nitrophenyl ester, hydroxypyridine ester, carbonyl imidazole, and the other such chemical groups. In some embodiments, the reactive functionality may be bonded directly to a nucleotide, or it may be bonded to the nucleotide through a linking group. The functional moiety and any linker cannot substantially impair the ability of the nucleotide to be added to the miRNA or to be labeled. Representative linking groups include carbon containing linking groups, typically ranging from about 2 to 18, usually from about 2 to 8 carbon atoms, where the carbon containing linking groups may or may not include one or more heteroatoms, e.g., S, O, N etc., and may or may not include one or more sites of unsaturation. Of particular interest in many embodiments, are alkyl linking groups, typically lower alkyl linking groups of 1 to 16, usually 1 to 4 carbon atoms, where the linking groups may include one or more sites of unsaturation.

In some embodiments, the present invention concerns miRNA that are labeled. It is contemplated that miRNA may first be isolated and/or purified prior to labeling. This may achieve a reaction that more efficiently labels the miRNA, as opposed to other RNA in a sample in which the miRNA is not isolated or purified prior to labeling. In many embodiments of the invention, the label is non-radioactive. Generally, nucleic acids may be labeled by adding labeled nucleotides (one-step process) or adding nucleotides and labeling the added nucleotides (two-step process). In some embodiments, nucleic acids are labeled by catalytically adding to the nucleic acid an already labeled nucleotide or nucleotides. One or more labeled nucleotides can be added to miRNA molecules. See U.S. Pat. No. 6,723,509, which is hereby incorporated by reference. In other embodiments, an unlabeled nucleotide or nucleotides is catalytically added to a miRNA, and the unlabeled nucleotide is modified with a chemical moiety that enables it to be subsequently labeled. In embodiments of the invention, the chemical moiety is a reactive amine such that the nucleotide is an amine-modified nucleotide. Examples of amine-modified nucleotides are well known to those of skill in the art, many being commercially available such as from Ambion, Sigma, Jena Bioscience, and TriLink.

Labels on miRNA or miRNA probes may be colorimetric (includes visible and UV spectrum, including fluorescent), luminescent, enzymatic, or positron emitting (including radioactive). The label may be detected directly or indirectly. Radioactive labels include ¹²⁵I, ³²P, ³³P, and ³⁵S. Examples of enzymatic labels include alkaline phosphatase, luciferase, horseradish peroxidase, and β-galactosidase. Labels can also be proteins with luminescent properties, e.g., green fluorescent protein and phicoerythrin.

The colorimetric and fluorescent labels contemplated for use as conjugates include, but are not limited to, Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and TOTAB. Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides are available from Molecular Probes, and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences, such as Cy3-UTP and Cy5-UTP.

Examples of fluorescently labeled deoxyribonucleotides include Dinitrophenyl (DNP)-11-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP.

It is contemplated that nucleic acids may be labeled with two different labels. Furthermore, fluorescence resonance energy transfer (FRET) may be employed in methods of the invention (e.g., Klostermeier et al., 2002; Emptage, 2001; Didenko, 2001, each incorporated by reference).

Alternatively, the label may not be detectable per se, but indirectly detectable or allowing for the isolation or separation of the targeted nucleic acid. For example, the label could be biotin, digoxigenin, polyvalent cations, chelator groups and the other ligands, include ligands for an antibody.

A number of techniques for visualizing or detecting labeled nucleic acids are readily available. Such techniques include, microscopy, arrays, Fluorometry, Light cyclers or other real time PCR machines, FACS analysis, scintillation counters, Phosphoimagers, Geiger counters, MRI, CAT, antibody-based detection methods (Westerns, immunofluorescence, immunohistochemistry), histochemical techniques, HPLC, spectroscopy, capillary gel electrophoresis, spectroscopy; mass spectroscopy; radiological techniques; and mass balance techniques.

When two or more differentially detectable labels are employed, fluorescent resonance energy transfer (FRET) techniques may be employed to characterize association of one or more nucleic acid. Furthermore, a person of ordinary skill in the art is well aware of ways of visualizing, identifying, and characterizing labeled nucleic acids, and accordingly, such protocols may be used as part of the invention. Examples of tools that may be used also include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule.

### Array Preparation and Screening

The present invention concerns the preparation and use of miRNA arrays or miRNA probe arrays useful for determining the expression of one or more miRNAs disclosed herein (e.g., SEQ ID NOS:1-160, Table 1, FIGS. 4A-4G). The arrays can be ordered macroarrays or microarrays of nucleic acid molecules (probes) that are fully or nearly complementary or identical to a plurality of miRNA molecules or precursor miRNA molecules and are positioned on a support or support material in a spatially separated organization. Macroarrays are typically a support (e.g., sheets of nitrocellulose or nylon) upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter.

Microarrays typically use coated glass as the solid support, in contrast to the nitrocellulose-based material of filter arrays. By having an ordered array of miRNA-complementing nucleic acid samples, the position of each sample can be tracked and linked to the original sample. A variety of different array devices in which a plurality of distinct nucleic acid probes are stably associated with the surface of a solid support are known to those of skill in the art. Useful substrates or supports for arrays include nylon, glass, metal, plastic, and silicon. Such arrays may vary in a number of different ways, including average probe length, sequence or types of probes, nature of bond between the probe and the array surface, e.g., covalent or non-covalent, and the like. The labeling and screening methods of the present invention and the arrays are not limited in its utility with respect to any parameter except that the probes detect miRNA; consequently, methods and compositions may be used with a variety of different types of miRNA arrays.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610;287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; W00138580; WO 0168255; WO 03020898; WO 03040410; WO 03053586; WO 03087297; WO 03091426; W003100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 2, 20, 25, 50, 80, 100 or more different probes. It is contemplated that they may contain 1000, 16,000, 65,000, 250,000 or 1,000,000 or more different probes. The probes can be directed to targets in one or more different organisms or cell types. The oligonucleotide probes range from 5 to 50, 5 to 45, 10 to 40, 9 to 34, or 15 to 40 nucleotides in length in some embodiments. In certain embodiments, the oligonucleotide probes are 5, 10, 15, 20 to 20, 25, 30, 35, 40 nucleotides in length including all integers and ranges there between.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm·sup·2. The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

### Sample Preparation

It is contemplated that the miRNA of a wide variety of samples can be analyzed using the arrays, index of miRNA probes, or array technology described herein and known to the skilled artisan. While endogenous miRNA is contemplated for use with compositions and methods of the invention, recombinant miRNA—including nucleic acids that are complementary or identical to endogenous miRNA or precursor miRNA—can also be handled and analyzed as described herein. Samples may be biological samples, in which case, they can be from biopsy, fine needle aspirates, exfoliates, scrappings, blood, tissue, organs, or any sample containing or constituting biological cells of interest. In certain embodiments, samples may be, but are not limited to, fresh, frozen, fixed, formalin fixed, paraffin embedded, or formalin fixed and paraffin embedded. Alternatively, the sample may not be a biological sample, but be a chemical mixture, such as a cell-free reaction mixture (which may contain one or more biological enzymes).

After an array or a set of miRNA probes is prepared and the miRNA in the sample is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in many embodiments is the use of stringent conditions during hybridization. Stringent conditions are known to those of skill in the art.

It is specifically contemplated that a single array or set of probes may be contacted with multiple samples. The samples may be labeled with different labels to distinguish the samples. Differences between the samples for particular miRNAs corresponding to probes on the array can be readily ascertained and quantified.

The small surface area of the array permits uniform hybridization conditions, such as temperature regulation and salt content. Moreover, because of the small area occupied by the high density arrays, hybridization may be carried out in extremely small fluid volumes (e.g., about 250 μl or less, including volumes of about or less than about 5, 10, 25, 50, 60, 70, 80, 90, 100 μl, or any range derivable therein). In small volumes, hybridization may proceed very rapidly.

### Differential Expression Analyses

Arrays of the invention can be used to detect differences in miRNA expression between two or more samples or between one or more samples and a reference miRNA expression profile. Specifically contemplated applications include identifying and/or quantifying differences between miRNA from a sample in question and that of a known such as a cancer cell line in which the responsiveness to an anti-cancer agent in question is known.

An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., Science, 1991, 251:767-777, each of which is incorporated by reference in its entirety for all purposes. These arrays may generally be produced using mechanical synthesis methods or light directed synthesis methods which incorporate a combination of photolithographic methods and solid phase synthesis methods. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes. Arrays may be packaged in such a manner as to allow for diagnostics or other manipulation of an all inclusive device, see for example, U.S. Pat. Nos. 5,856,174 and 5,922,591 incorporated in their entirety by reference for all purposes. See also U.S. patent application Ser. No. 09/545,207, filed Apr. 7, 2000 for additional information concerning arrays, their manufacture, and their characteristics, which is incorporated by reference in its entirety for all purposes.

Cancers that may be evaluated and treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, fallopian tube, prostate, skin, CNS, stomach, testis, or tongue.

Cancers of any organ or tissue can be evaluated or treated, including but not limited to colon, pancreas, breast, prostate, bone, liver, kidney, lung, testes, skin, pancreas, stomach, colorectal cancer, renal cell carcinoma, hepatocellular carcinoma, melanoma, etc. Examples of breast cancer include, but are not limited to, invasive ductal carcinoma, invasive lobular carcinoma, ductal carcinoma in situ, and lobular carcinoma in situ. Examples of cancers of the respiratory tract include, but are not limited to, small-cell and non-small-cell lung carcinoma, as well as bronchial adenoma and pleuropulmonary blastoma. Examples of brain cancers include, but are not limited to, brain stem and hypothalamic glioma, cerebellar and cerebral astrocytoma, medulloblastoma, ependymoma, as well as neuroectodermal and pineal tumor. Tumors of the male reproductive organs include, but are not limited to, prostate and testicular cancer. Tumors of the female reproductive organs include, but are not limited to, endometrial, cervical, ovarian, vaginal, and vulvar cancer, as well as sarcoma of the uterus. Tumors of the digestive tract include, but are not limited to, anal, colon, colorectal, esophageal, gallbladder, gastric, pancreatic, rectal, small-intestine, and salivary gland cancers. Tumors of the urinary tract include, but are not limited to, bladder, penile, kidney, renal pelvis, ureter, and urethral cancers. Eye cancers include, but are not limited to, intraocular melanoma and retinoblastoma. Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma. Skin cancers include, but are not limited to, squamous cell carcinoma, Kaposi's sarcoma, malignant melanoma, Merkel cell skin cancer, and non-melanoma skin cancer. Head-and-neck cancers include, but are not limited to, laryngeal, hypopharyngeal, nasopharyngeal, and/or oropharyngeal cancers, and lip and oral cavity cancer. Lymphomas include, but are not limited to, AIDS-related lymphoma, non-Hodgkin's lymphoma, cutaneous T-cell lymphoma, Hodgkin's disease, and lymphoma of the central nervous system. Sarcomas include, but are not limited to, sarcoma of the soft tissue, osteosarcoma, malignant fibrous histiocytoma, lymphosarcoma, and rhabdomyosarcoma. Leukemias include, but are not limited to, acute myeloid leukemia, acute lymphoblastic leukemia, chronic lymphocytic leukemia, chronic myelogenous leukemia, and hairy cell leukemia.

In some embodiments the malignancy is in the form of a solid tumor. In other embodiments, the malignancy is in the form of a non-solid tumor (having dispersed cancer cells), such as leukemia, lymphoma, or other blood malignancies.

In certain embodiments, miRNA profiles may be generated to evaluate and correlate those profiles with pharmacokinetics. For example, miRNA profiles may be created and evaluated for a subject's tumor and blood samples prior to the subject's being treated or during treatment to determine if there are miRNAs whose level of expression (elevated or decreased) relative to a reference miRNA expression level correlates with the clinical outcome of the subject. Identification of differential miRNAs can lead to a diagnostic assay involving them that can be used to evaluate tumor and/or blood samples to determine what drug regimen the subject should be provided. In addition, it can be used to identify or select subjects suitable for a particular clinical trial. If a miRNA profile is determined to be correlated with drug efficacy or drug toxicity that may be relevant to whether that patient is an appropriate subject for receiving the drug or for a particular dosage of the drug.

### Therapeutic Methods

Methods of the invention include reducing or eliminating activity of one or more miRNAs in a cell in vitro or in vivo, comprising introducing into a cell an miRNA inhibitor; or supplying or enhancing the activity of one or more miRNAs in a cell. The present invention also concerns inducing certain cellular characteristics by providing to a cell a particular nucleic acid, such as a specific synthetic miRNA molecule or a synthetic miRNA inhibitor molecule. However, in methods of the invention, the miRNA molecule or miRNA inhibitor need not be synthetic. They may have a sequence that is identical to a naturally occurring miRNA or they may not have any design modifications. In certain embodiments, the miRNA molecule and/or an miRNA inhibitor are synthetic, as discussed herein.

The particular nucleic acid molecule provided to the cell is understood to correspond to a particular miRNA in the cell, and thus, the miRNA in the cell is referred to as the “corresponding miRNA.” In situations in which a named miRNA molecule is introduced into a cell, the corresponding miRNA will be understood to be the induced miRNA. It is contemplated, however, that the miRNA molecule introduced into a cell is not a mature miRNA but is capable of becoming a mature miRNA under the appropriate physiological conditions. In cases in which a particular corresponding miRNA is being inhibited by a miRNA inhibitor, the particular miRNA will be referred to as the targeted miRNA. It is contemplated that multiple corresponding miRNAs may be involved. In particular embodiments, more than one miRNA molecule is introduced into a cell. Moreover, in other embodiments, more than one miRNA inhibitor is introduced into a cell. Furthermore, a combination of miRNA molecule(s) and miRNA inhibitor(s) may be introduced into a cell.

Methods include identifying a cell or subject in need of inducing those cellular characteristics. Also, it will be understood that an amount of an agent (for example, an anti-cancer agent or nucleic acid) that is provided to a cell or organism is an “effective amount,” which refers to an amount needed to achieve a desired goal, such as inducing a particular cellular characteristic(s).

In certain embodiments of the methods include providing or introducing to a cell a nucleic acid molecule corresponding to a mature miRNA in the cell in an amount effective to achieve a desired physiological result.

Moreover, methods can involve providing synthetic or nonsynthetic miRNA molecules. It is contemplated that in these embodiments, methods may or may not be limited to providing only one or more synthetic miRNA molecules or only one or more nonsynthetic miRNA molecules. Thus, in certain embodiments, methods may involve providing both synthetic and nonsynthetic miRNA molecules. In this situation, a cell or cells are most likely provided a synthetic miRNA molecule corresponding to a particular miRNA and a nonsynthetic miRNA molecule corresponding to a different miRNA.

In some embodiments, the treatment methods are methods for reducing or inhibiting cell proliferation in a cell comprising introducing into or providing to the cell an effective amount of (i) an miRNA inhibitor molecule or (ii) a synthetic or nonsynthetic miRNA molecule that corresponds to an miRNA sequence. In certain embodiments the methods involves introducing into the cell an effective amount of (i) an miRNA inhibitor molecule having a 5′ to 3′ sequence that is at least 90% complementary to all or part of the 5′ to 3′ sequence of one or more mature miRNA of SEQ ID NO:1-160 or Table 1.

Typically, an endogenous gene, miRNA or mRNA is modulated in the cell. In particular embodiments, the nucleic acid sequence comprises at least one segment that is at least 70, 75, 80, 85, 90, 95, or 100% identical in nucleic acid sequence to one or more miRNA sequence listed in Table 1. Modulation of the expression or processing of an endogenous gene, miRNA, or mRNA can be through modulation of the processing of an mRNA, such processing including transcription, transportation and/or translation with in a cell. Modulation may also be effected by the inhibition or enhancement of miRNA activity with a cell, tissue, or organ. Such processing may effect the expression of an encoded product or the stability of the mRNA. In still other embodiments, a nucleic acid sequence can comprise a modified nucleic acid sequence.

Methods of the invention can further comprise administering a second therapy, such as chemotherapy, radiotherapy, surgery, immunotherapy, or a combination of two or more of the foregoing. The nucleic acid can be transcribed from a viral vector or a nucleic acid vector, such as a plasmid vector or other non-viral vector.

In certain aspects, a subject is administered: one or more nucleic acids possessing a function of an miRNA having a nucleic acid segment having at least 80, 85, 90, 95, 97, 98, 99, or 100% nucleic acid sequence identity to those miRNA decreased or down-regulated in a disease or condition to be treated, wherein the decreased miRNA is associated with (correlates with) resistance to an anti-cancer agent in a corresponding cancer cell line.

In certain aspects, a subject is administered: one or more miRNA inhibitors having a nucleic acid segment having at least 80, 85, 90, 95, 97, 98, 99, or 100% nucleic acid sequence identity to those miRNA increased or up-regulated in a disease or condition to be treated.

Synthetic nucleic acids can be administered to the subject or patient using modes of administration that are well known to those of skill in the art, particularly for therapeutic applications. It is particularly contemplated that a patient is human or any other mammal.

It will be understood in methods of the invention that a cell or other biological matter such as an organism (including patients) can be provided an miRNA or miRNA molecule corresponding to a particular miRNA by administering to the cell or organism a nucleic acid molecule that functions as the corresponding miRNA once inside the cell. The form of the molecule provided to the cell may not be the form that acts as an miRNA once inside the cell. Thus, it is contemplated that in some embodiments, biological matter is provided a synthetic miRNA or a nonsynthetic miRNA, such as one that becomes processed into a mature and active miRNA once it has access to the cell's miRNA processing machinery. In certain embodiments, it is specifically contemplated that the miRNA molecule provided to the biological matter is not a mature miRNA molecule but a nucleic acid molecule that can be processed into the mature miRNA once it is accessible to miRNA processing machinery. The term “nonsynthetic” in the context of miRNA means that the miRNA is not “synthetic,” as defined herein. Furthermore, it is contemplated that in embodiments of the invention that concern the use of synthetic miRNAs, the use of corresponding nonsynthetic miRNAs is also considered an aspect of the invention, and vice versa.

In certain embodiments, methods also include targeting an miRNA to modulate in a cell or organism. The term “targeting an miRNA to modulate” or “targeting an miRNA” means a nucleic acid of the invention will be employed so as to modulate the selected miRNA. In some embodiments the modulation is achieved with a synthetic or non-synthetic miRNA that corresponds to the targeted miRNA, which effectively provides the targeted miRNA to the cell or organism (positive modulation). In other embodiments, the modulation is achieved with an miRNA inhibitor, which effectively inhibits the targeted miRNA in the cell or organism (negative modulation).

In certain embodiments, the miRNA is targeted because an anti-cancer agent can be made more effective in the subject by negative modulation of the targeted miRNA in the subject. In other embodiments, the miRNA is targeted because an anti-cancer agent can be made more effective in the subject by positive modulation of the targeted miRNA in the subject.

In certain methods of the invention, there is a further step of administering the selected miRNA modulator to a cell, tissue, organ, or organism (collectively “biological matter”) in need of treatment related to modulation of the targeted miRNA or in need of the physiological or biological results discussed herein (such as with respect to a particular cellular pathway or result, such as a decrease in cell viability). Consequently, in some methods of the invention there is a step of identifying a subject in need of treatment that can be provided by the miRNA modulator(s). It is contemplated that an effective amount of an miRNA modulator can be administered in some embodiments. In particular embodiments, there is a therapeutic benefit conferred on the biological matter, where a “therapeutic benefit” refers to an improvement in the one or more conditions or symptoms associated with a disease or condition or an improvement in the prognosis, duration, or status with respect to the disease. It is contemplated that a therapeutic benefit includes, but is not limited to, a decrease in pain, a decrease in morbidity, a decrease in a severity or duration of a symptom. For example, with respect to cancer, it is contemplated that a therapeutic benefit can be inhibition of tumor growth, prevention of metastasis, reduction in number of metastases, inhibition of cancer cell proliferation, inhibition of cancer cell proliferation, induction of cell death in cancer cells, inhibition of angiogenesis near cancer cells, induction of apoptosis of cancer cells, reduction in pain, reduction in risk of recurrence, induction of chemo- or radiosensitivity in cancer cells, prolongation of life, palliation of symptoms related to the condition, and/or delay of death directly or indirectly related to a cancer.

Furthermore, it is contemplated that the miRNA compositions may be provided as part of a therapy to a patient, in conjunction with traditional therapies or preventative agents. Moreover, it is contemplated that any method discussed in the context of therapy may be applied as a preventative measure, particularly in a patient identified to be potentially in need of the therapy or at risk of the condition or disease for which a therapy is needed.

In addition, methods of the invention concern employing one or more nucleic acids corresponding to an miRNA and an anti-cancer agent (e.g., a chemotherapeutic drug). The nucleic acid can enhance the effect or efficacy of the anti-cancer agent, reduce any side effects or toxicity, modify its bioavailability, and/or decrease the dosage or frequency needed. In certain embodiments, the therapeutic drug is a cancer therapeutic. Consequently, in some embodiments, there is a method of treating cancer in a subject comprising administering to the subject the cancer therapeutic and an effective amount of at least one miRNA molecule that improves the efficacy of the cancer therapeutic or protects non-cancer cells. Cancer therapies also include a variety of combination therapies with both chemical and radiation based treatments. Combination chemotherapies include but are not limited to, for example, bevacizumab, cisplatin (CDDP), carboplatin, EGFR inhibitors (gefitinib and cetuximab), procarbazine, mechlorethamine, cyclophosphamide, camptothecin, COX-2 inhibitors (e.g., celecoxib) ifosfamide, melphalan, chlorambucil, busulfan, nitrosurea, dactinomycin, daunorubicin, doxorubicin (adriamycin), bleomycin, plicomycin, mitomycin, etoposide (VP16), tamoxifen, raloxifene, estrogen receptor binding agents, taxol, taxotere, gemcitabien, navelbine, farnesyl-protein transferase inhibitors, transplatinum, 5-fluorouracil, vincristin, vinblastin and methotrexate, or any analog or derivative variant of the foregoing.

Generally, inhibitors of miRNAs can be given to achieve the opposite effect as compared to when nucleic acid molecules corresponding to the mature miRNA are given. Similarly, nucleic acid molecules corresponding to the mature miRNA can be given to achieve the opposite effect as compared to when inhibitors of the miRNA are given.

### Kits

Any of the compositions described herein may be comprised in a kit. In a non-limiting example, reagents for isolating miRNA, labeling miRNA, and/or evaluating a miRNA population using an array, nucleic acid amplification, and/or hybridization can be included in a kit, as well reagents for preparation of samples. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. In certain aspects, the kit can include amplification reagents. In other aspects, the kit may include various supports, such as glass, nylon, polymeric beads, and the like, and/or reagents for coupling any probes and/or target nucleic acids. It may also include one or more buffers, such as reaction buffer, labeling buffer, washing buffer, or a hybridization buffer, compounds for preparing the miRNA probes, and components for isolating miRNA. Other kits of the invention may include components for making a nucleic acid array comprising miRNA, and thus, may include, for example, a solid support.

Kits for implementing methods of the invention described herein are specifically contemplated. In some embodiments, there are kits for preparing miRNA for multi-labeling and kits for preparing miRNA probes and/or miRNA arrays. In these embodiments, kit comprise, in suitable container means, 1, 2, 3, 4, 5, 6, 7, 8, 9, 10, 11, 12 or more of the following: (1) poly(A) polymerase; (2) unmodified nucleotides (G, A, T, C, and/or U); (3) a modified nucleotide (labeled or unlabeled); (4) poly(A) polymerase buffer; (5) at least one microfilter; (6) label that can be attached to a nucleotide; (7) at least one miRNA probe; (8) reaction buffer; (9) a miRNA array or components for making such an array; (10) acetic acid; (11) alcohol; and (12) solutions for preparing, isolating, enriching, and purifying miRNAs or miRNA probes or arrays. Other reagents include those generally used for manipulating RNA, such as formamide, loading dye, ribonuclease inhibitors, and DNAse.

In specific embodiments, kits of the invention include an array containing miRNA probes, as described in the application. An array may have probes corresponding to all known miRNAs of an organism or a particular tissue or organ in particular conditions, or to a subset of such probes. The subset of probes on arrays of the invention may be or include those identified as relevant to a particular diagnostic, therapeutic, or prognostic application. For example, the array may contain one or more probes that is indicative or suggestive of (1) a disease or condition, (2) susceptibility or resistance to a particular drug or treatment; (3) susceptibility to toxicity from a drug or substance; (4) the stage of development or severity of a disease or condition (prognosis); and (5) genetic predisposition to a disease or condition.

For any kit embodiment, including an array, there can be nucleic acid molecules that contain or can be used to amplify a sequence that is a variant of, identical to or complementary to all or part of any of SEQ ID NOS: 1-157. In certain embodiments, a kit or array of the invention can contain one or more probes for the miRNAs identified by SEQ ID NOS:1-157. Any nucleic acid discussed above may be implemented as part of a kit.

The components of the kits may be packaged either in aqueous media or in lyophilized form. The container means of the kits will generally include at least one vial, test tube, flask, bottle, syringe or other container, into which a component may be placed, and preferably, suitably aliquotted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits of the invention. The dye may then be re-suspended in any suitable solvent, such as DMSO.

The container(s) will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

Such kits may also include components that facilitate isolation of the labeled miRNA. It may also include components that preserve or maintain the miRNA or that protect against its degradation. Such components may be RNAse-free or protect against RNAses. Such kits generally will comprise, in suitable means, distinct containers for each individual reagent or solution.

Kits of the invention may also include one or more of the following: control RNA; nuclease-free water; RNAse-free containers, such as 1.5 ml tubes; RNAse-free elution tubes; PEG or dextran; ethanol; acetic acid; sodium acetate; ammonium acetate; guanidinium; detergent; nucleic acid size marker; RNAse-free tube tips; and RNAse or DNAse inhibitors.

It is contemplated that such reagents are embodiments of kits of the invention. Such kits, however, are not limited to the particular items identified above and may include any reagent used for the manipulation or characterization of miRNA.

All patents, patent applications, provisional applications, and publications referred to or cited herein, supra or infra, are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### MATERIALS AND METHODS FOR EXAMPLES 1-3

Cell culture. A subset of 40 of the NCI60 cancer cell line panel was obtained from the NCI developmental Therapeutics Program (FIG. 3). Cryopreserved cells were thawed rapidly in a 37° C. water bath, suspended in 10 ml of RPMI1640 (GIBCO) with L-glutamine (2 mM, GIBCO) and 10% fetal bovine serum (FBS; Sigma) centrifuged (400 g/7 minutes), and re-suspended in 1 ml of the above medium. Cell viability was assessed by trypan blue exclusion and cells were seeded a density of 5 to 8×10⁶. All cultures were incubated at 37° C., 5% CO₂ for 2-3 days with fresh medium added on the second day. Cell lines underwent one passage before total RNA extraction.

Total RNA extraction and microarray analysis. Total RNA was extracted from 1×10⁶ log phase cells using mirVana miRNA Isolation kit (Ambion) according to the manufacturer's instructions. The yield and quality of total RNA for each cell line was determined using an Agilent bioanalyzer. 10 μg of total RNA from each cell line then was subject to miRNA expression profiling. The 40 cell line samples were co-hybrized to printed arrays that contained a 562 Ambion mirVana miRNA probe set (Ambion, Austin Tex.) and 632 of Invitrogen's NCode multispecies miRNA probes (Invitrogen, Carlsbad Calif.), which contain 335 unique human miRNAs. The hybridized arrays were scanned on a GenePix 4000B scanner and expression data was generated using the GenePix Pro software (Molecular Devices, Sunnydale Calif.). All protocols are able to be found on the website genome.duke.edu/cores/microarray/services/spotted-arrays/protocols/.

miR-367 Transfection. Pre-miR-367 and Anti-miR-367 were purchased from Ambion. Log-phase cells (˜60% confluency) were transfected with 6.25 μM Pre-miR-367 or Anti-miR-367 using siPORT NeoFX transfection reagent (Ambion) at a final concentration of 50 nM according to manufacturer's instructions. Controls included transfection of a non-targeting miRNA (Negative Control #1).

Real-time Quantitative RT-PCR of miRNA. Real-time Quantitative RT-PCR was used to validate successful miRNA transfection. TaqMan®MicroRNA Reverse Transcription kit (Applied Biosystems) was used to convert specific miRNAs to cDNA. RNU44 was used as control for quantification. Per Applied Biosystems' protocol, real-time PCR was performed adding 1.34 μl of each completed RT reaction to target TaqMan microRNA Assay reaction with TaqMan Universal PCR Master Mix (final reaction volume equal 20 μl). Samples (786-0 and TK-10 cell lines transfected with Pre-miR-367 precursor, Anti-miR-367 inhibitor, and Anti-miR-inhibitor Negative Control #1) were evaluated in triplicate and run on the Applied Biosystems 7900HT Fast Real-Time PCR System. Assay data was analyzed using SDS 2.2.2 software.

Growth inhibition assay. Topotecan, obtained from Sequola Research Products Ltd, was dissolved in DMSO (Sigma) at concentration of 20 mM and stored at −20° C. Cells were seeded in 96-well plates (Nunc) at a density of 5×10⁵ cells per ml and incubated overnight at 37° C. Cells were incubated with the indicated concentrations of topotecan for 48 hours and cell viability was analyzed using the CellTiter-Glo™ luminescent cell viability assay kit (Promega). Luminescence was recorded using Wallac Victor²™ 1420 Multilabel Counter (Perkin Elmer Life Sciences). Wells containing medium without cells were used to obtain background luminescence. All experimental wells and controls were set up in triplet.

Statistical analysis. Two-color spotted array data for miRNA were generated from the GenePix Pro software. Background subtraction [17] and Loess normalization [18] were performed using Limma software. Replicate probe sets were averaged by their design—Ambion or Invitrogen. Processed data were then analyzed using SAM (Significance Analysis of Microarrays) software [19]. Missing values were imputed with SAM's k-nearest neighbor imputer, where k was set to 10. For each drug, Pearson's Correlation test was used to identify those miRNAs with expression values associated with sensitivity measured by GI50.

Pathway Analysis. Messenger RNA genes that are predicted targets of miRNAs associated with in vitro sensitivity in the inventors' analysis were identified using the miRDB database. In an effort to place miRNA and gene expression data into a relevant biologic context, an analysis of biologic pathway relationships was performed using commercially available software (MetaCore from GeneGo Inc systems). This literature-curated application correlates gene expression array data to relevant biological pathways, such that one can identify the networks, molecular mechanisms, and biological processes most relevant to developed data. A p-value of <0.05 was used to determine the statistical significance of the association between the predicted target genes of miRNA mentioned above and biologically relevant pathways identified in this analysis.

### Example 1

### MicroRNA Expression and Cancer Cell Line GI50

Based on the inventors' generated miRNA expression data and publicly available GI50 values for cisplatin, carboplatin, doxorubicin, paclitaxel, docetaxel, gemcitabine and topotecan, Pearson's correlation test identified 22 miRNAs associated with cisplatin sensitivity (p<0.05), 48 miRNAs associated with doxorubicin sensitivity (p<0.05), 35 miRNAs associated with sensitivity to paclitaxel, topotecan (p<0.05), 34 miRNAs associated with sensitivity to gemcitabine (p<0.05), 32 miRNAs associated with docetaxel sensitivity (p<0.05), and 29 miRNAs associated with carboplatin sensitivity (p<0.05) (FIGS. 4A-G). Sixteen miRNAs (miR367, miR200c, miR515, miR377, miR508, miR340, miR129, miR130a, miR142_5p, miR155, miR296, miR34c, miR380_5p, miR489, miR494, and miR526) were associated with in vitro sensitivity to 3 or more anti-cancer agents (FIGS. 4A-G). Of the 16 miRNAs found to correlate with resistance to 3 or more anti-cancer agents, 13 miRNAs were correlated with topotecan sensitivity, 13 with gemcitabine sensitivity, 8 with docetaxel sensitivity, 6 with cisplatin and paclitaxel sensitivity, 5 with doxorubicin sensitivity and 2 with carboplatin sensitivity.

### Example 2

### The Effect of Altered MicroRNA Level on Renal Carcinoma Cell Viability

In an effort to validate the biological significance of the inventors' findings, and to evaluate miRNAs associated with drug sensitivity as potential therapeutic targets, the inventors selected miR-367 and topotecan to be studied further. This miRNA/drug combination was selected based on the p-value observed in the correlation analysis of topotecan GI50 and miR-367 expression (p=0.0035), and the extremes of levels seen in sensitive and resistant cells available for further analysis. With this in mind, renal cell lines 786-0 and TK-10 were selected for analysis based on their associations between topotecan sensitivity and miR-367 expression: Renal cell line 786-0 showed the highest expression value of miR-367 (1.072) and the highest sensitivity to topotecan (log₁₀ GI₅₀ value of −7.903), and conversely, renal cell line TK-10 showed the lowest expression value of miR-367 (−2.84) and the lowest sensitivity to topotecan (log₁₀ GI₅₀ −5.279). These cell lines were therefore used in further experiments focused on manipulation of miR-367 levels and evaluation of effect on topotecan-sensitivity.

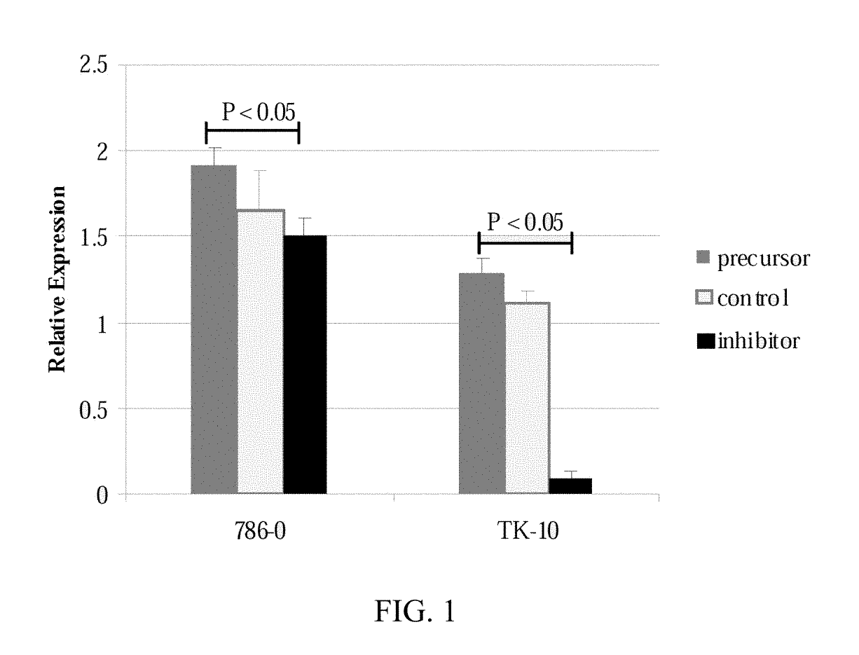

The 786-0 and TK-10 cell lines were transfected with the precursor (Pre-miR-367) or the inhibitor (Anti-miR-367) of miR-367 and evaluated for changes in sensitivity to topotecan. Transfection of the Pre-miR-367 and the Anti-miR-367 in the 786-0 and TK-10 cell lines resulted in an increase and decrease in miR-367 levels, respectively, in cell lines when compared to the non-targeting miRNA control (FIG. 1).

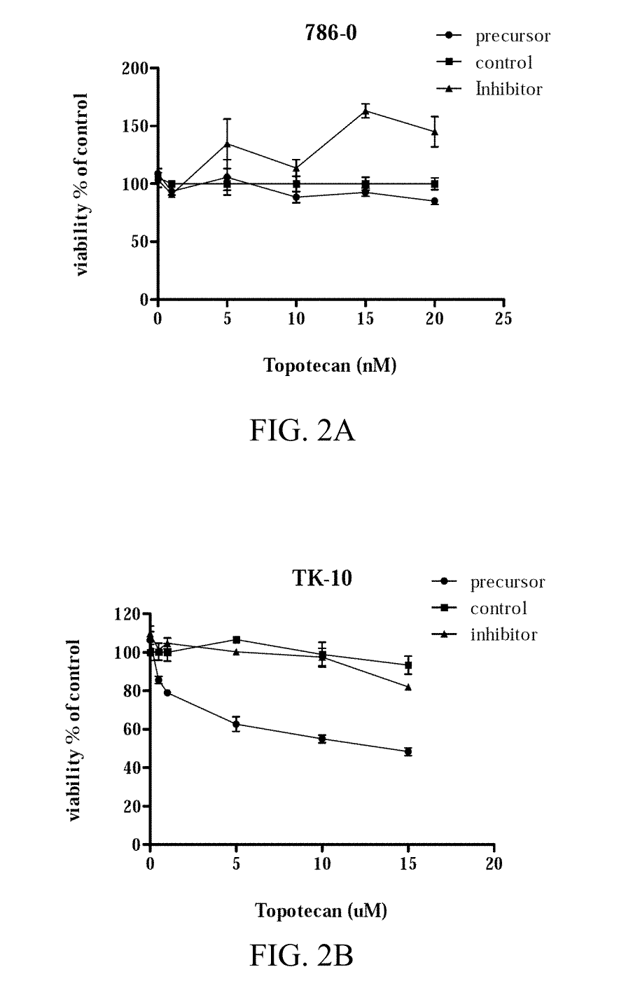

The effects of miR-367 modulation on topotecan-induced cell death and growth arrest were evaluated using the CellTiter-Glo' luminescent cell viability assay (FIG. 2). Twenty-four hours after transfection, cells were subject to 48 hours of topotecan treatment. As expected, depletion of miR-367 in the intrinsically sensitive cell line, 786-0 (high miR-367), decreased sensitivity to topotecan-induced growth arrest when compared to non-targeting control transfected cells. No differences in topotecan sensitivity were observed with over-expression of miR-367 in these cells. In contrast, over-expression of miR-367 in the intrinsically resistant cell line, TK-10 (low miR-367), significantly increased cell death and growth arrest induced by topotecan (two tail t-test: P value=0.0134). Consistently, no differences in topotecan sensitivity were observed with inhibition of miR-367 in these cells.

### Example 3

### Pathways Involved in De-Regulated MicroRNAs

In an effort to gain some insights into the potential role of identified miRNAs on various cellular processes, the inventors endeavored to identify the predicted mRNA targets genes of miR-367 using the miRDB database [20]. This database hosts 703 human miRNAs with 236,543 gene targets. In doing so, the inventors identified 435 predicted mRNA target genes for miR-367, with 68 of these predicted target genes having a prediction score more than 80. In an effort to place these predicted target genes into a relevant biologic context, an analysis of biologic pathway relationships was performed using commercially available software (GeneGo systems). Pathway modeling identified 9 pathways represented in miR-367 predicted target genes (p<0.05), the top four of which (by p-value) are associated with control of apoptosis and cell survival including cytoplasmic/mitochondrial transport of the proapoptotic proteins Bid, Bmf and Bim (p<0.003) and also APRIL and BAFF signaling (p=0.003).

### MATERIALS AND METHODS FOR EXAMPLE 4

Cell culture. Ovarian cancer cell lines ChicisR, SKOV4, and PA1 were obtained from Dr. Susan Murphy (Duke University Durham N.C.), ovarian cancer cell line OVCAR4 and breast cancer cell lines MCF-7 and Hs578T were obtained from NCI Developmental Therapeutics Program (DTP). The culture method is the same as that described in the Materials and Methods for Examples 1-3.

miR-367, miR-302b and miR-30a-5p Transfection. Pre-miR-367 precursor and anti-miR-367 inhibitor, pre-miR-302b precursor and anti-miR-302b inhibitor, and pre-miR-30a-5p precursor and anti-miR-30a-5p inhibitor were purchased from Applied Systems Inc., the method of transfection for all three miRNAs was the same as described in the Materials and Methods for Examples 1-3. The fold change between transfected and negative control groups was presented as log₁₀ 2^{-ΔΔCt}.

Real-time Quantitative RT-PCR of miRNA. A Real-Time relative quantity RT-PCR was used to validate successful miRNA transfection. TaqMan MicroRNA Reverse Transcription kit (Applied Biosystems) was used to convert specific miRNAs to cDNA. RNU44 was used as endogenous miRNA control for normalization. Per Applied Biosystems' protocol, real-time PCR was performed by adding 1.33 ul of each completed RT reaction to target TaqMan® microRNA Assay reaction with TaqMan Universal PCR Master Mix (final reaction volume equal 20 ul). Samples were evaluated in triplicate and run on the Applied Biosystems StepOne RT-PCR system. Assay data was analyzed by the instrument software.

Growth inhibition assay. Paclitaxel and topotecan, obtained from Sigma and Sequoia Research Products Ltd, were dissolved in DMSO (Sigma) at a concentration of 100 mM and stored at −20° C. Transfected cells were seeded in 96-well plates (Perkin Elmer) at a density of 5×10⁴ cells per ml and incubated overnight at 37° C. Cells were then incubated with a series of concentrations (6 concentrations at a dilution of 1:2) of paclitaxel or topotecan for 72 hours. Cell viability was analyzed using the CellTiter-Glo™ luminescent cell viability kit (Promega). Luminescence was recorded using Wallac Victor²™ 1420 multilabel Counter (Perkin Elmer life Sciences). Wells containing medium without cells were used to obtain background luminescence. All experimental wells and controls were set up in triplet. Dose-response curve was made using GraphPad (GraphPad Prism, version 5.02). P value was calculated by comparing EC50 between transfected and negative control groups using Sigmoidal dose-response curve (variable slope) equation.

### Example 4

### The Effect of Altered MicroRNA Level on Ovarian and Breast Cancer Cell Viability

In order to further evaluate miRNAs associated with drug sensitivity as potential therapeutic targets, the inventors selected the ovarian cancer cell lines ChicisR, OVCAR4, SKOV4, and PA1, the breast cancer cell lines Hs578T and MCF-7, the anti-cancer drugs paclitaxel and topotecan, and the miRNAs miR302b, miR367, and miR30a5p to be studied. The six cell lines were selected based on the GI50 of paclitaxel and topotecan. These ovarian and breast cancer cell lines were used in experiments focused on manipulation of miR302b, miR367, and miR30a5p levels and evaluation of effect on paclitaxel-sensitivity and topotecan-sensitivity. The three miRNAs (miR302b, miR367, and miR30a5p) were selected for transfection with the precursor miRNA or inhibitor based on the correlation between GI50 and miRNA expression:
miR302b, miR367: low expression—resistance to drug
miR30a5p: high expression—resistance to drug

The ovarian and breast cell lines were transfected with the precursor (Pre-miR-302b, Pre-miR-367, or Pre-miR-30a5p; Ambion) or the inhibitor (Anti-miR-302b, Anti-mir-367, or Anti-miR-30a5p; Ambion) of miR302b, miR367, and miR30a5p and evaluated for changes in sensitivity to paclitaxel and topotecan.

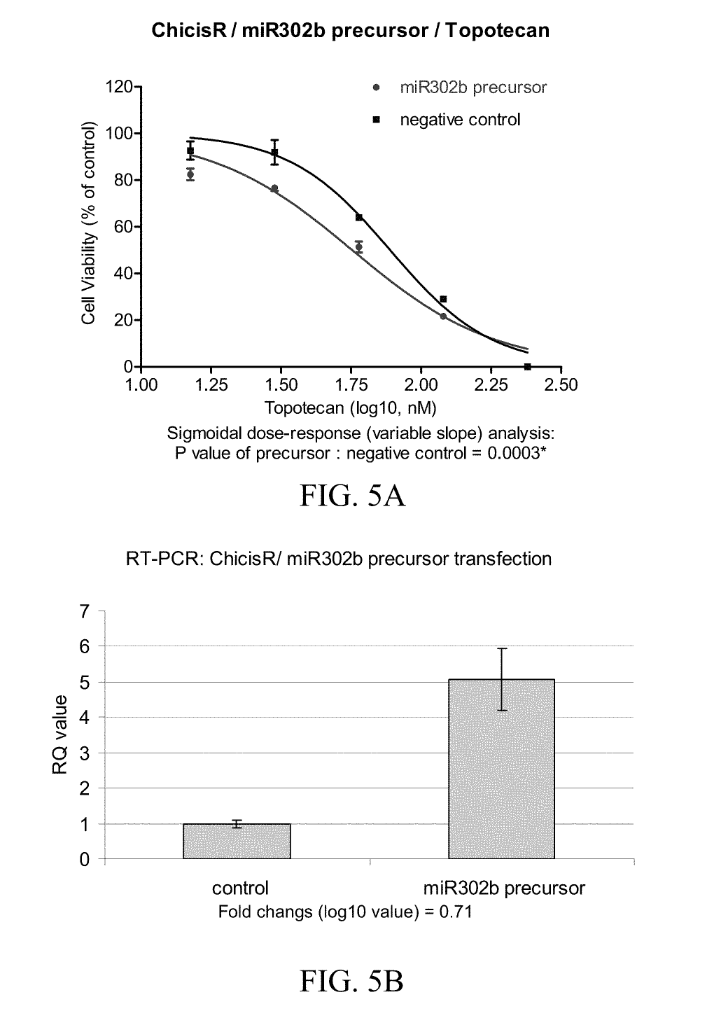

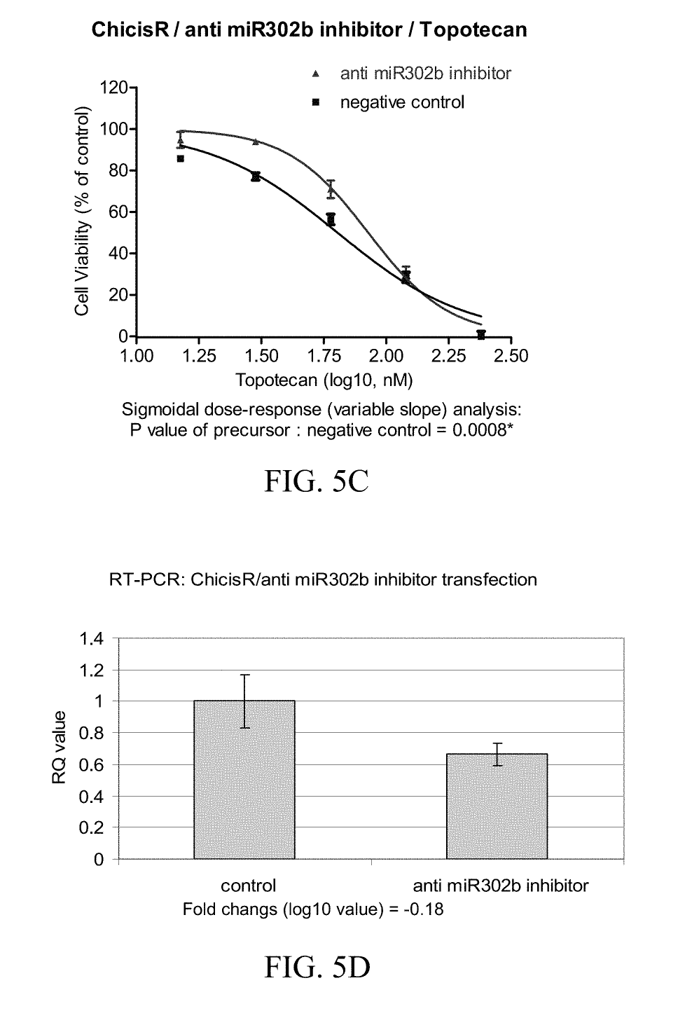

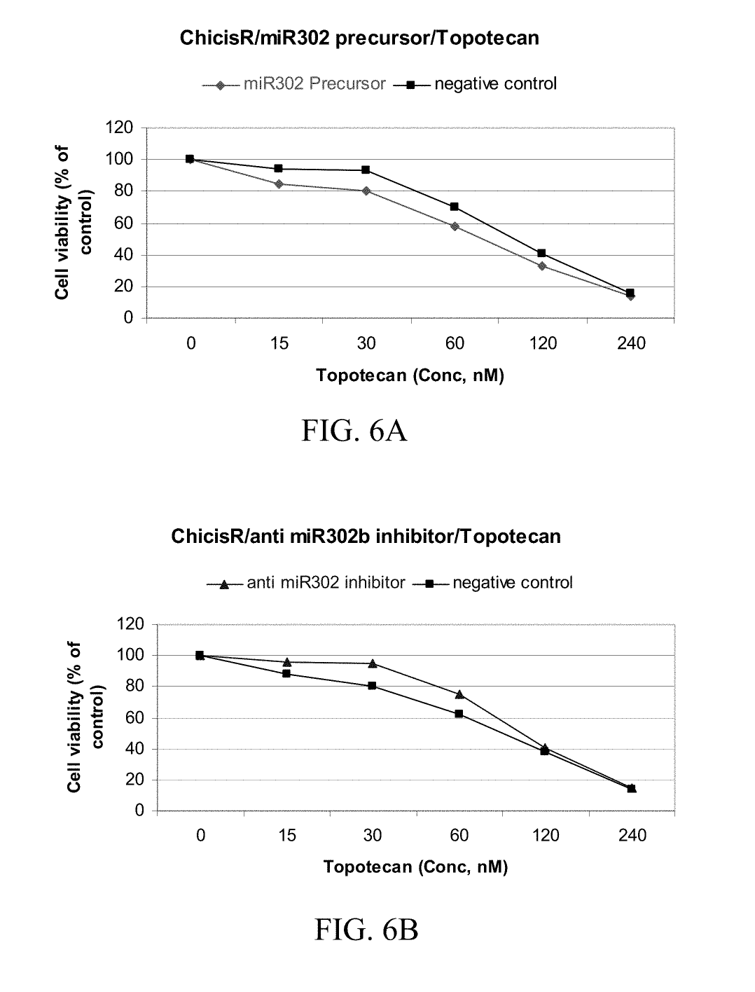

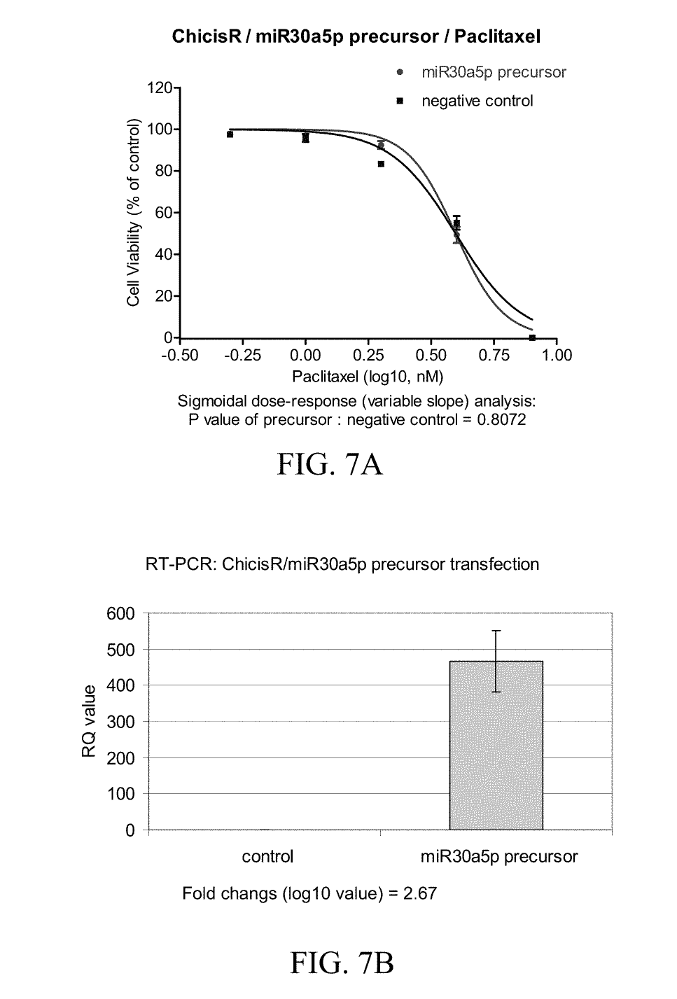

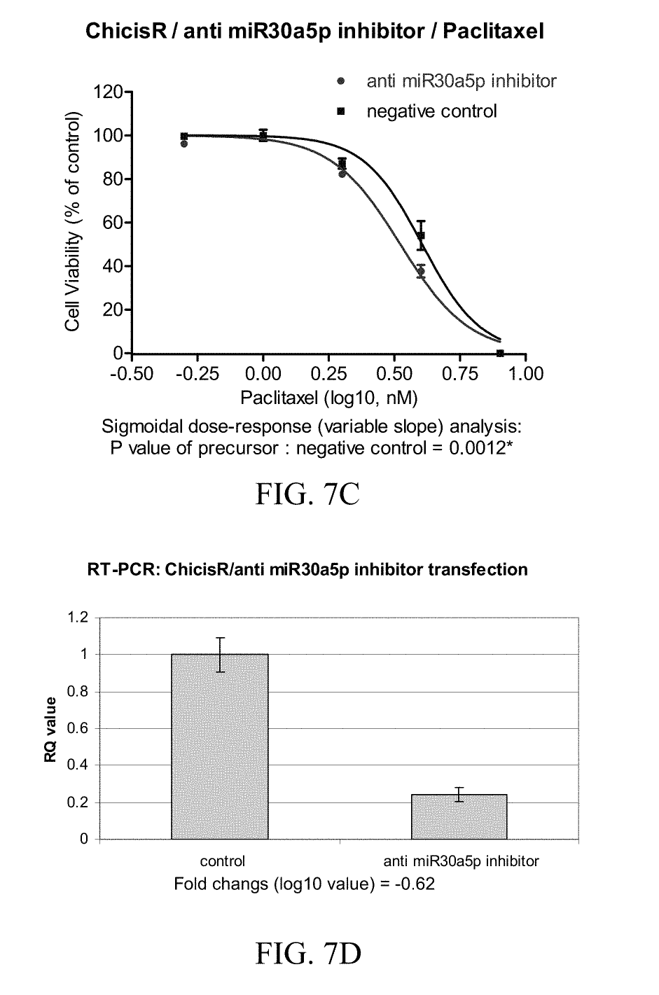

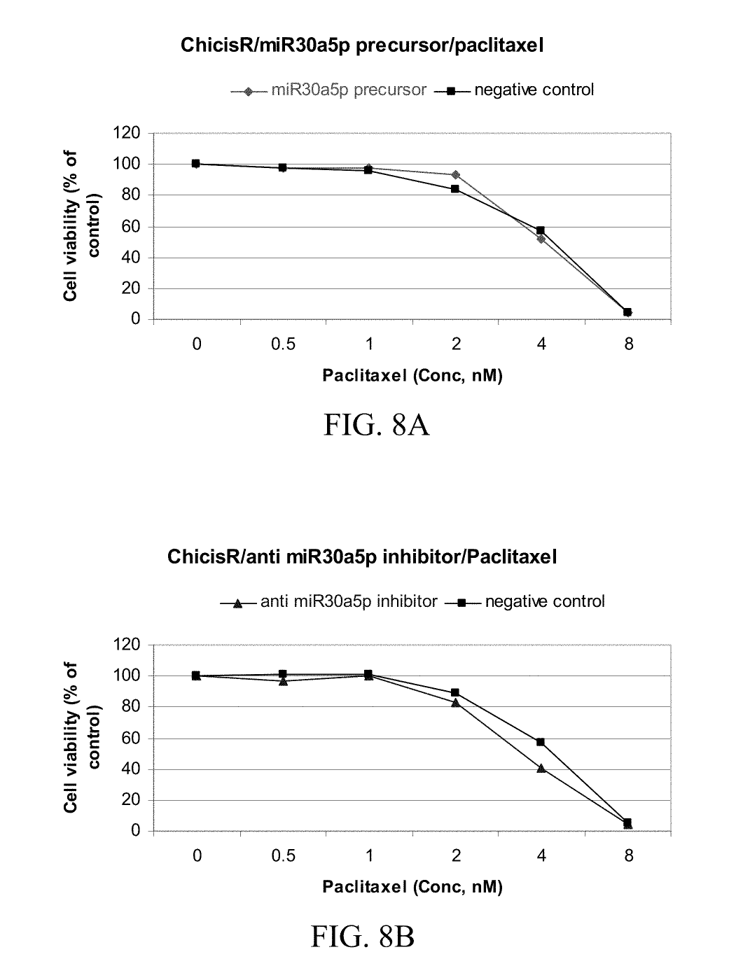

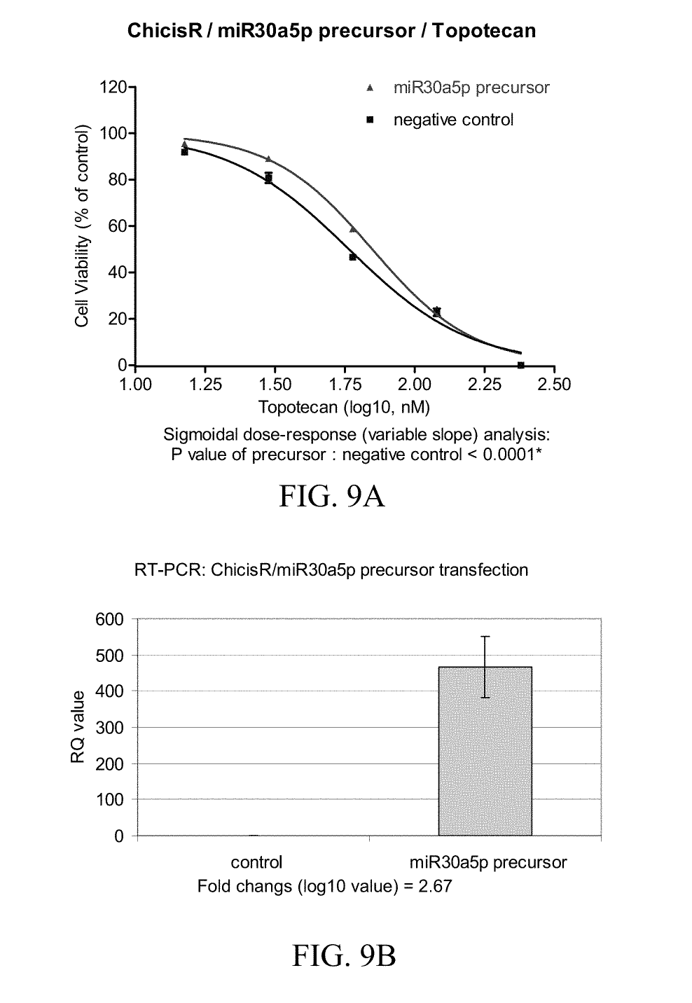

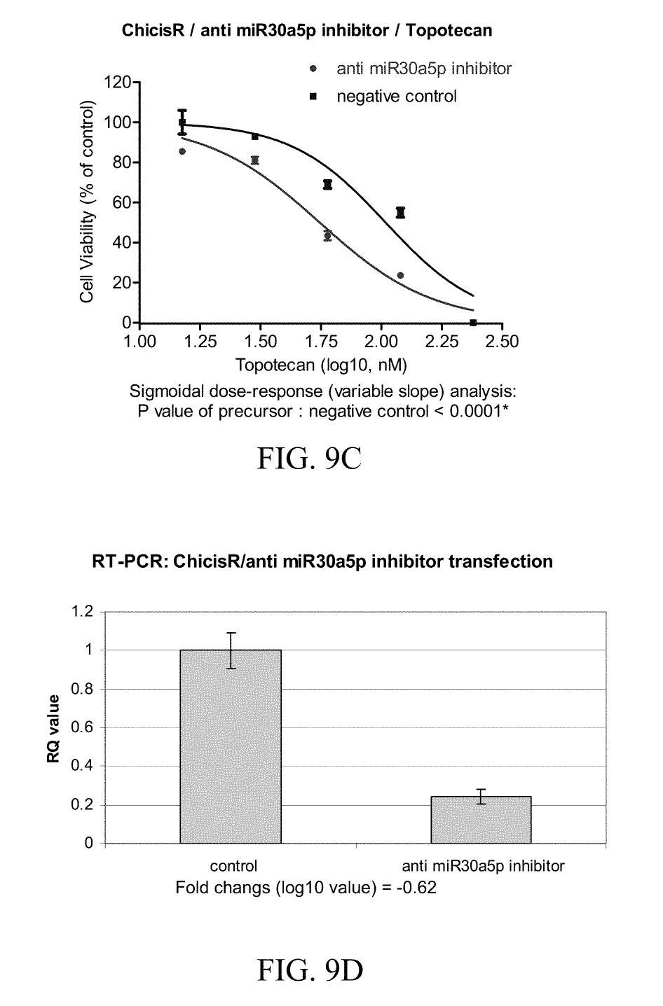

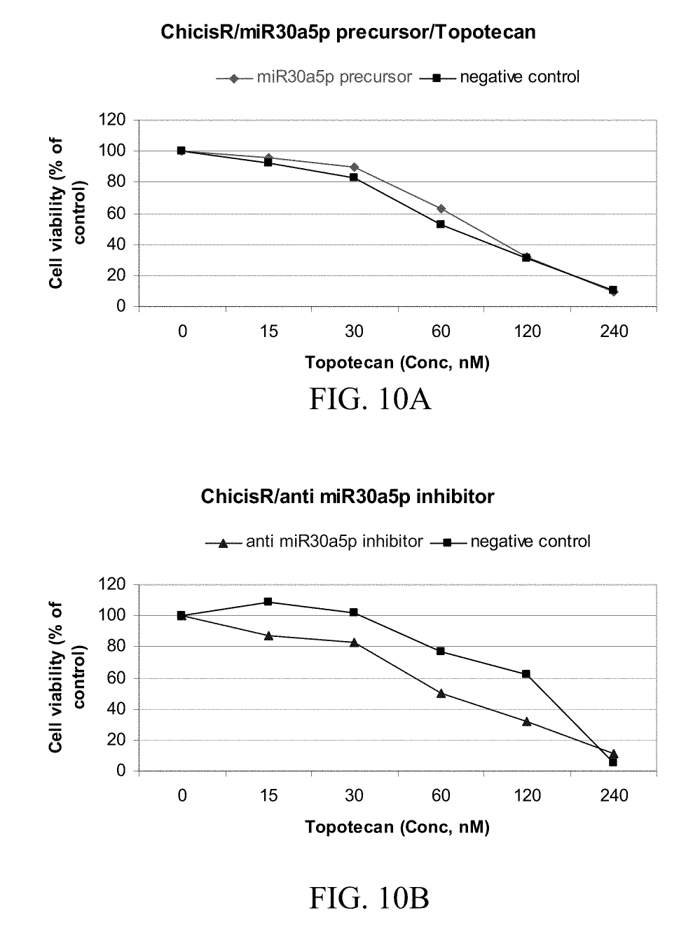

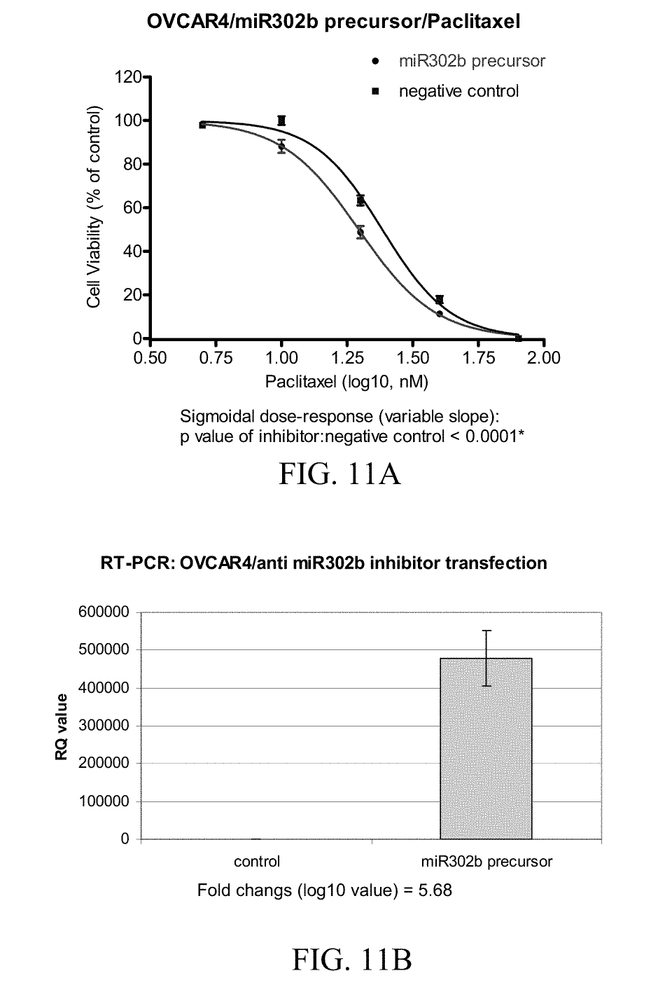

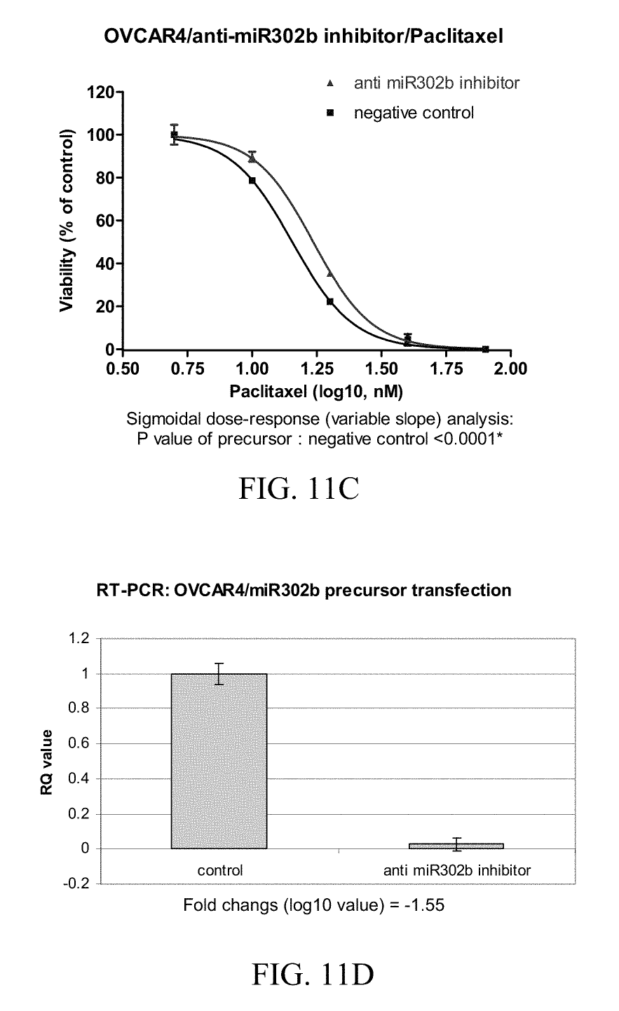

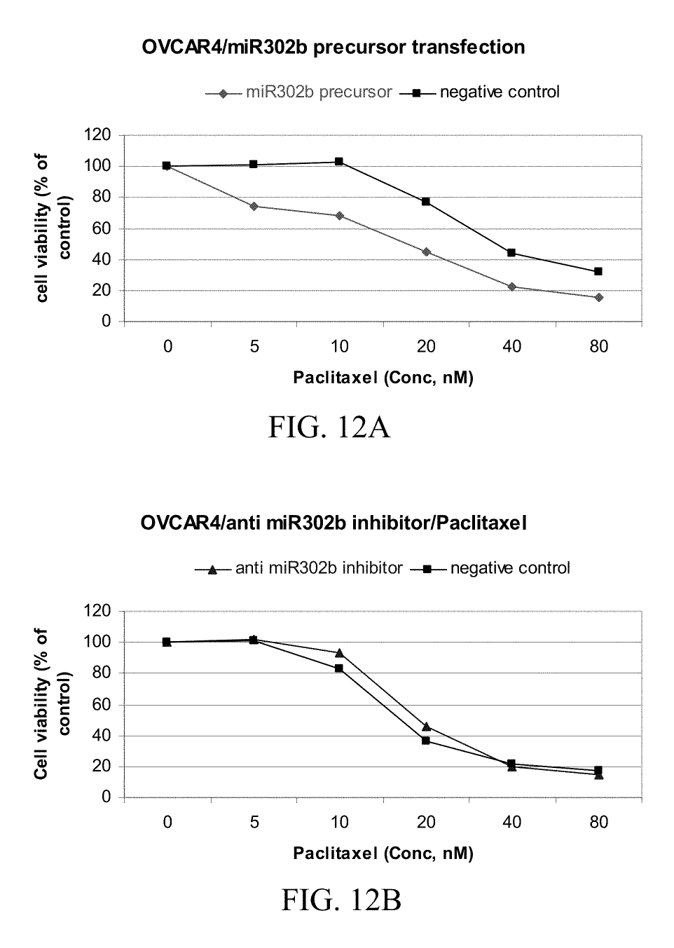

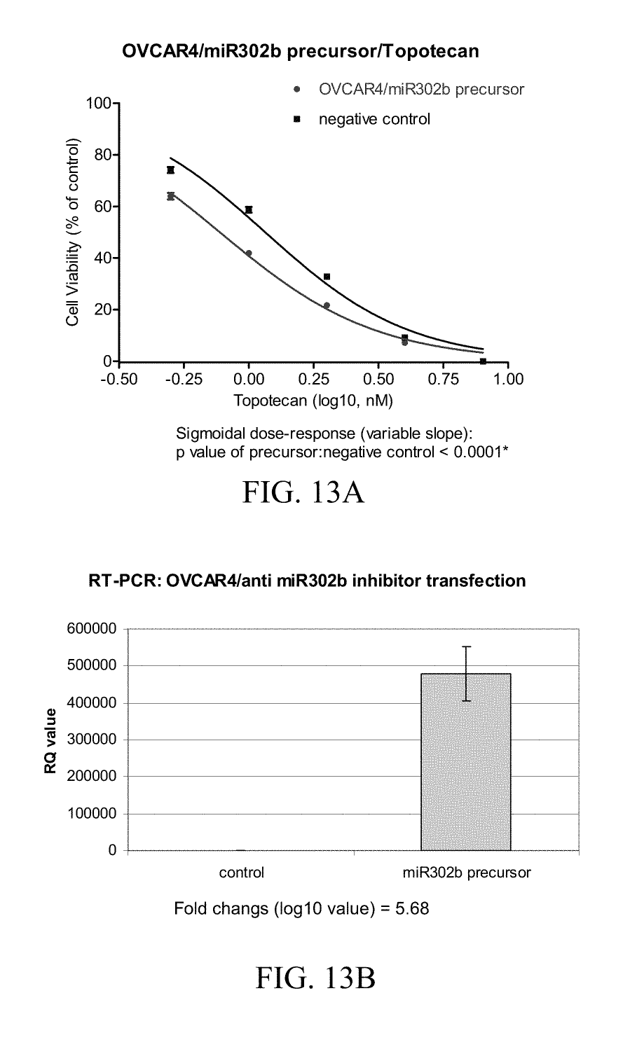

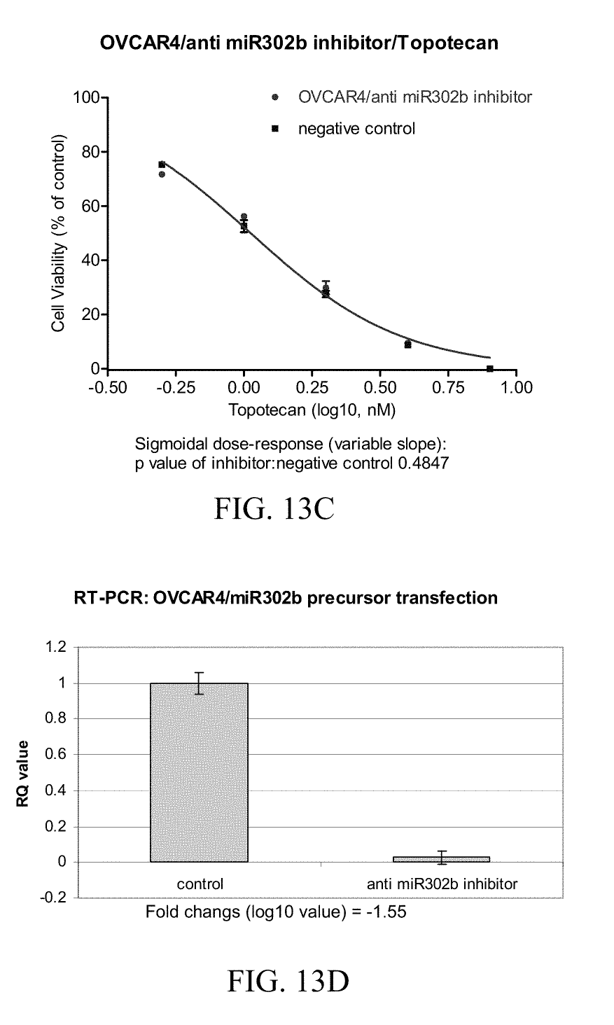

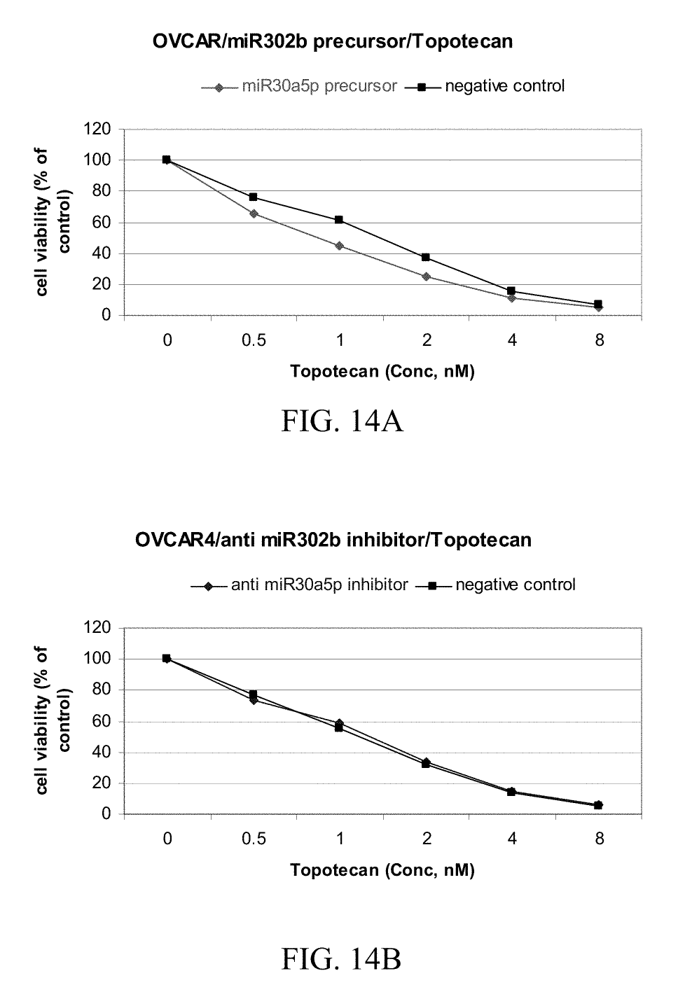

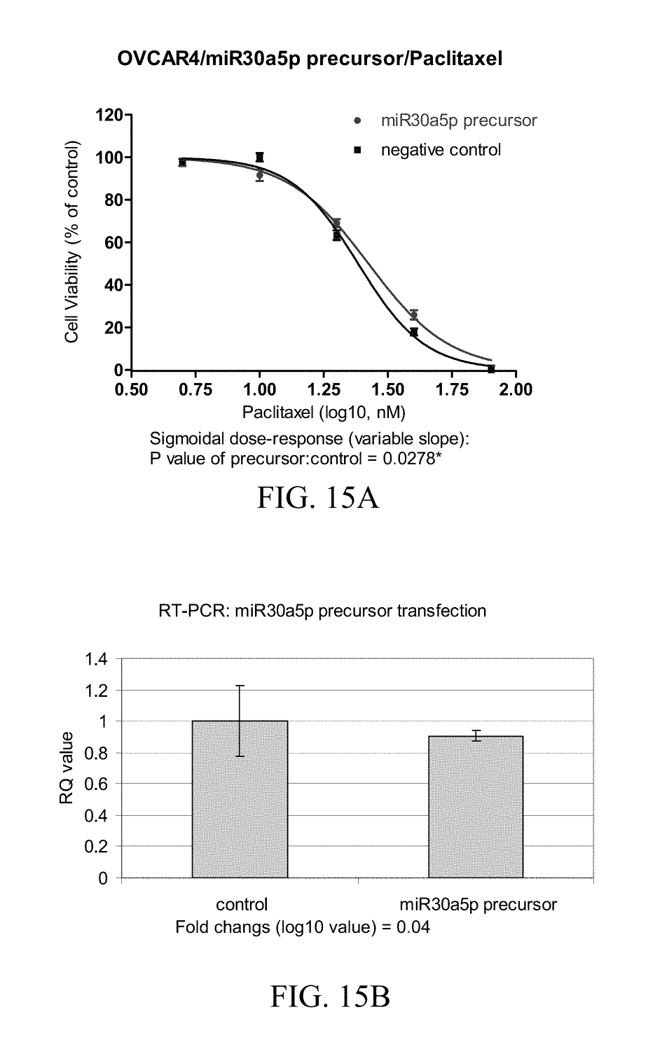

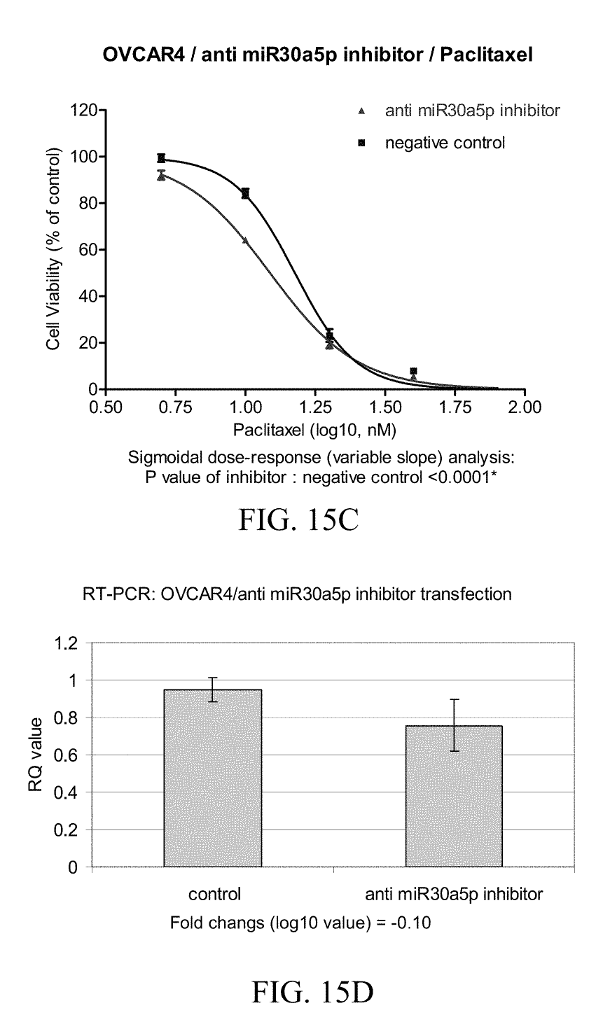

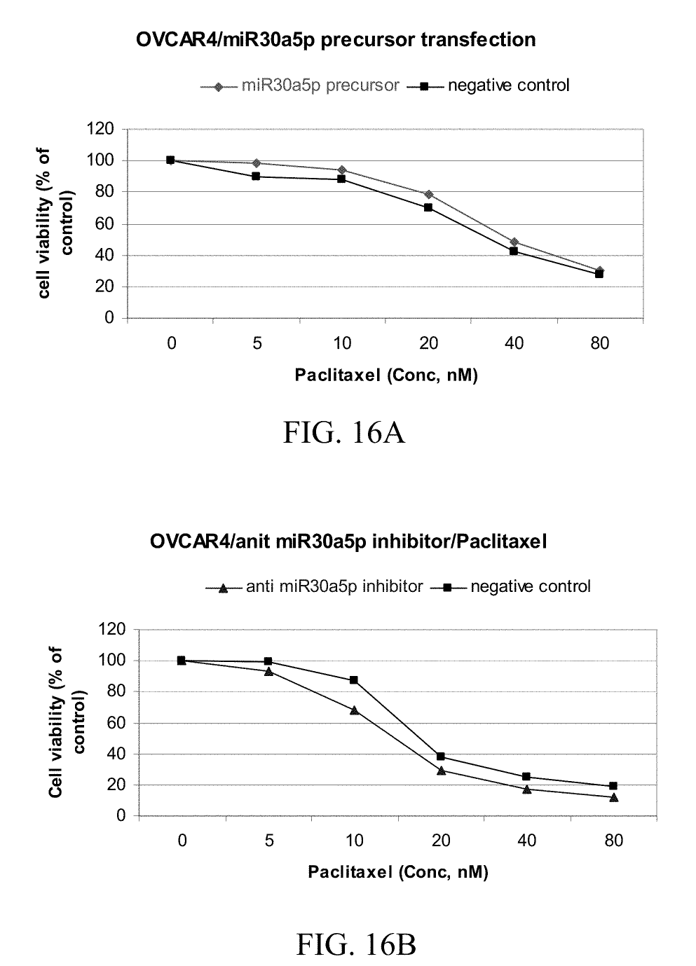

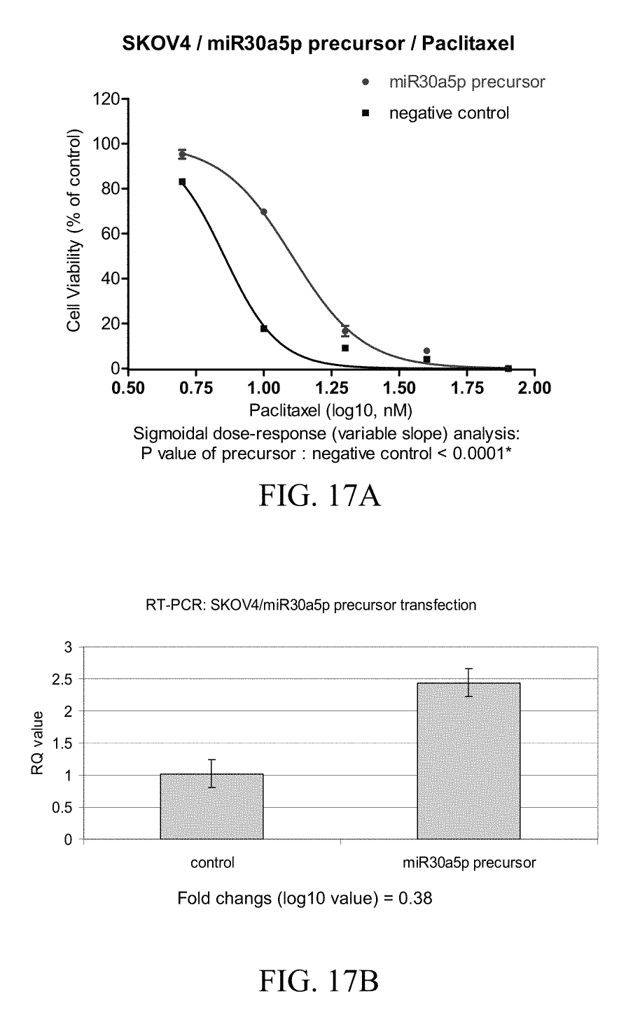

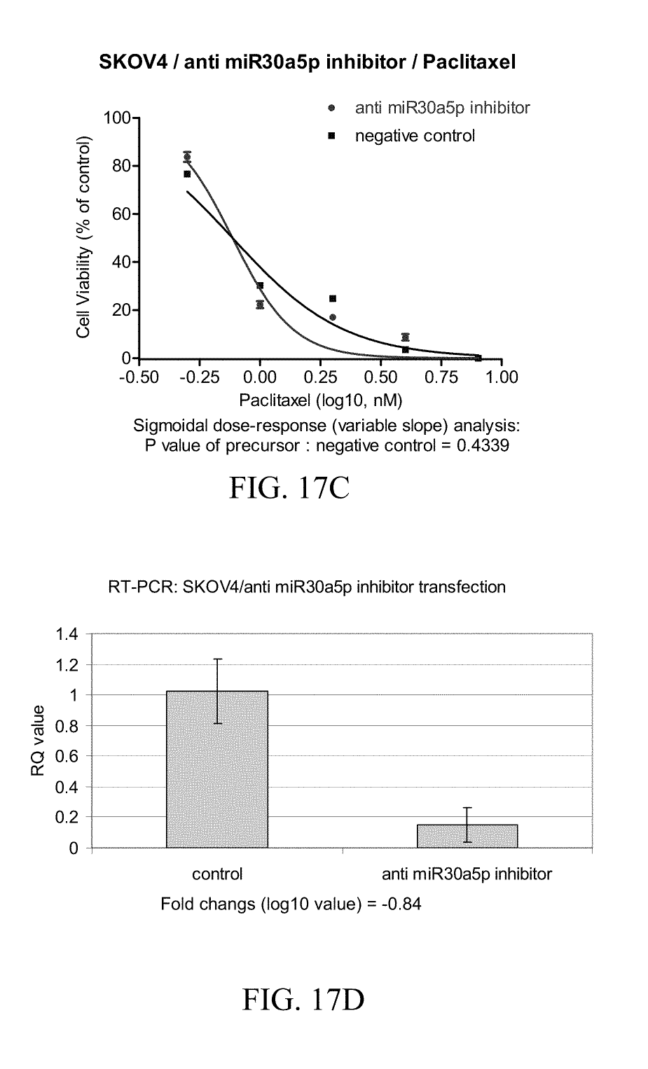

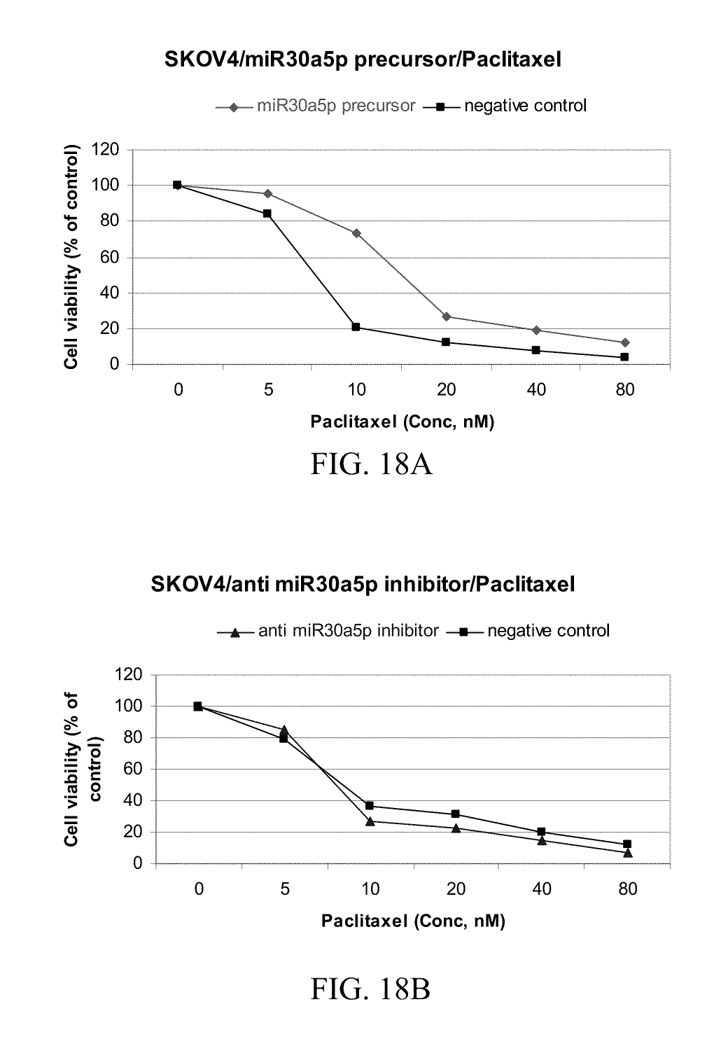

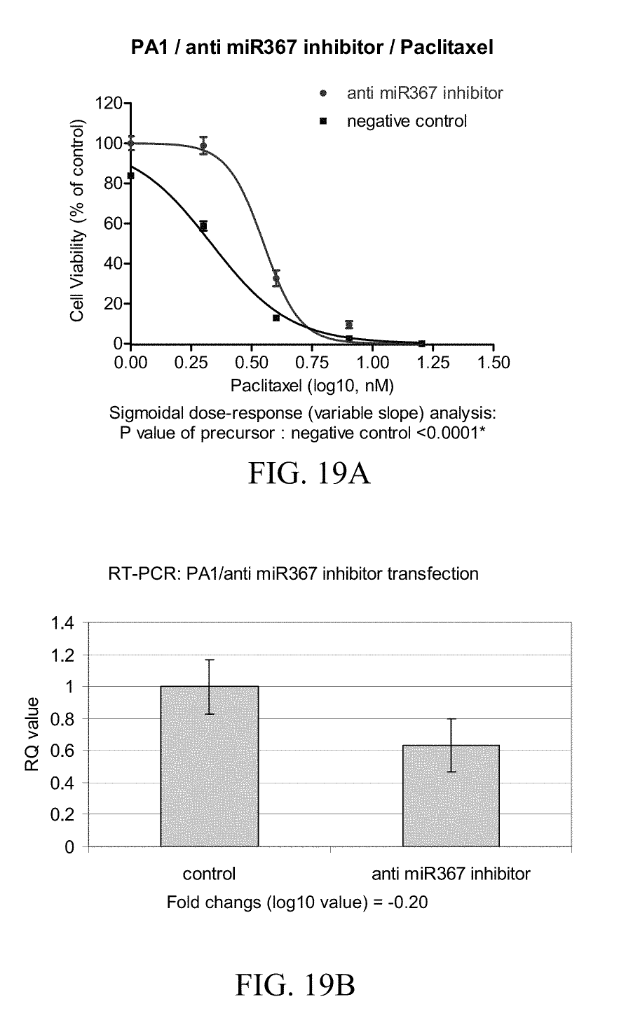

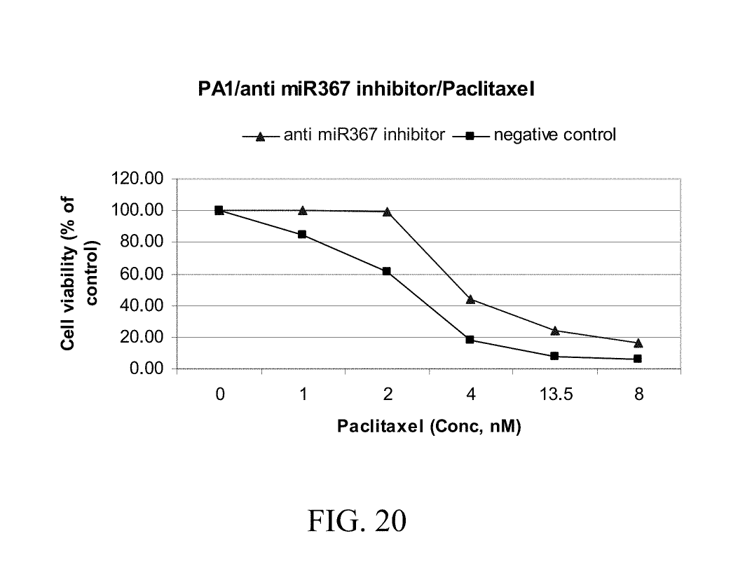

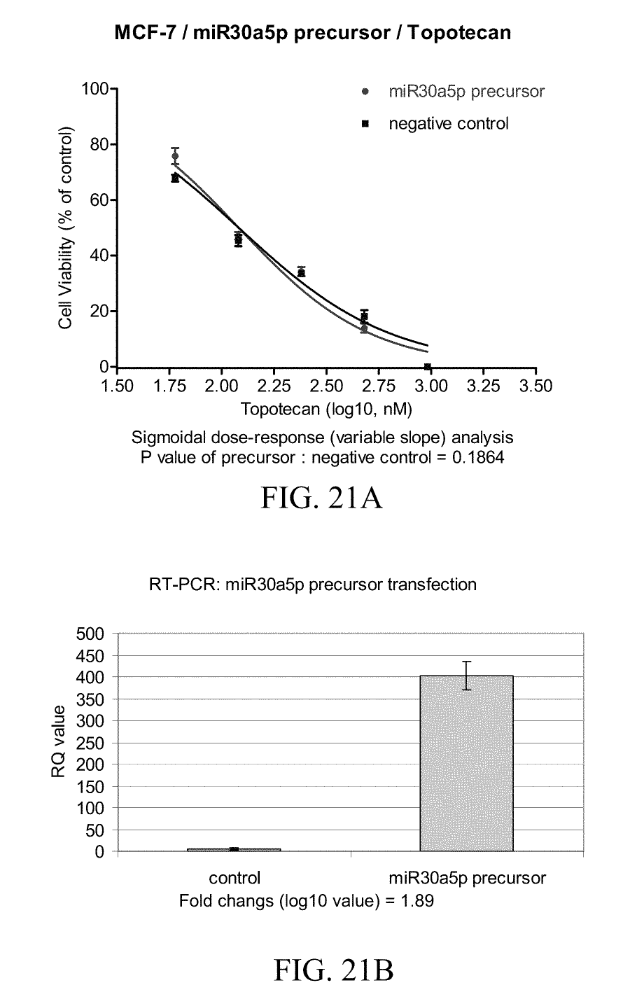

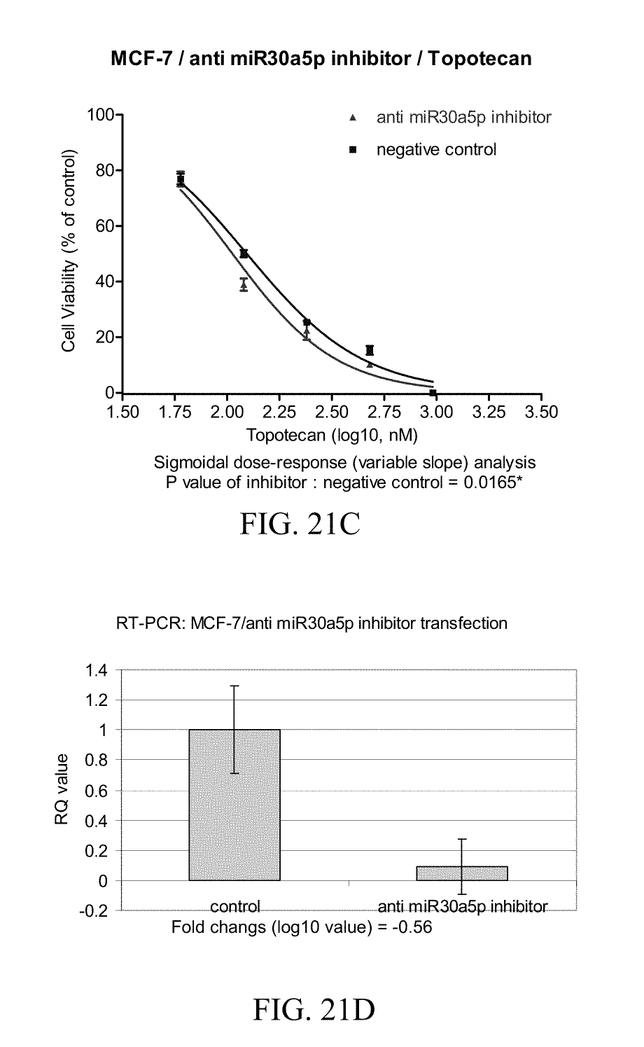

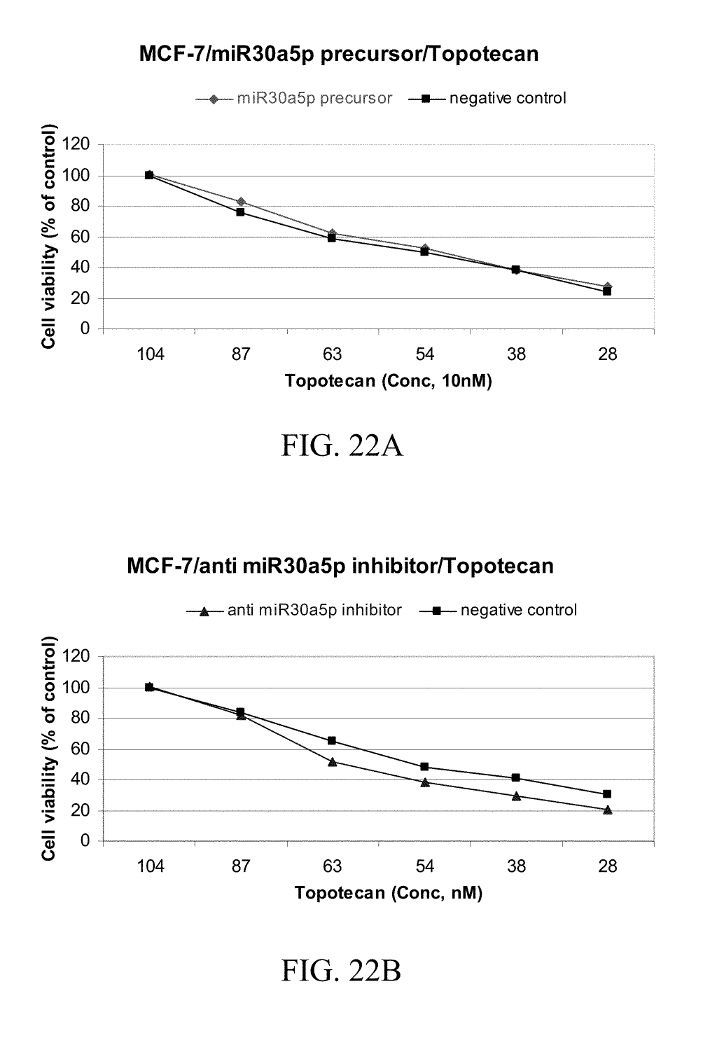

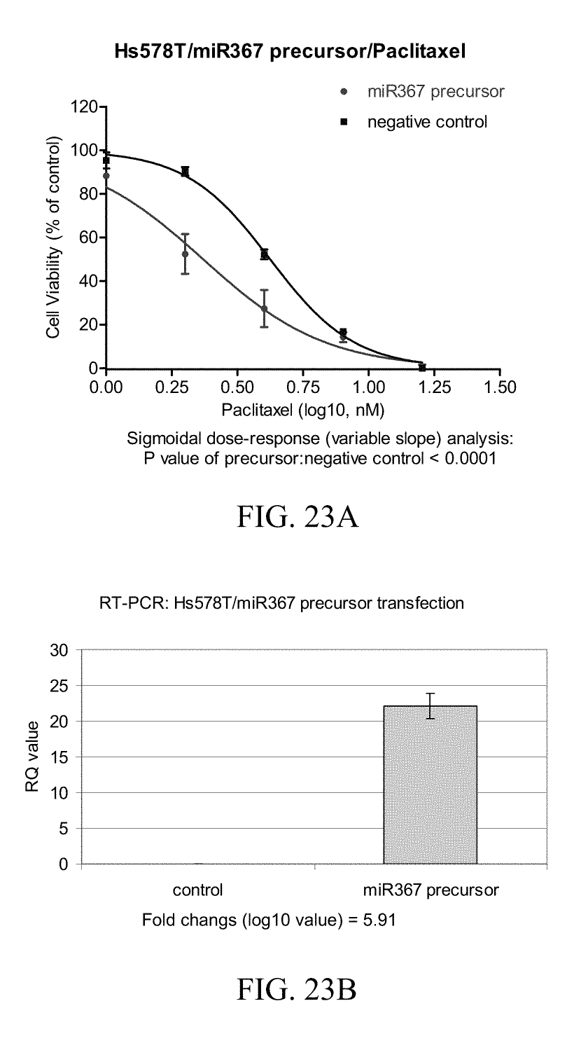

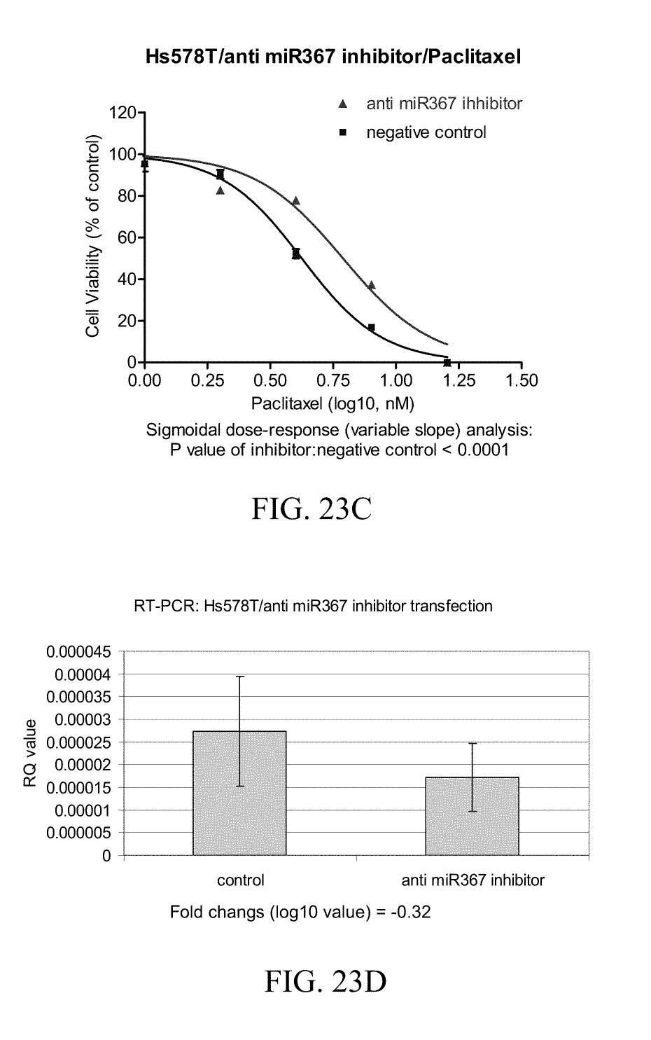

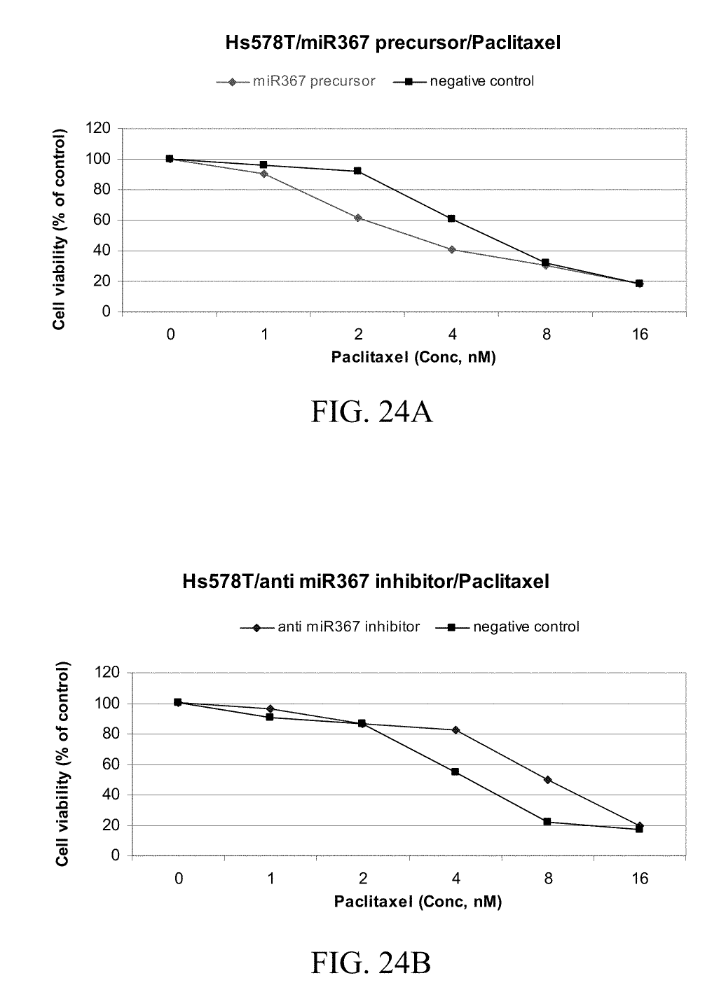

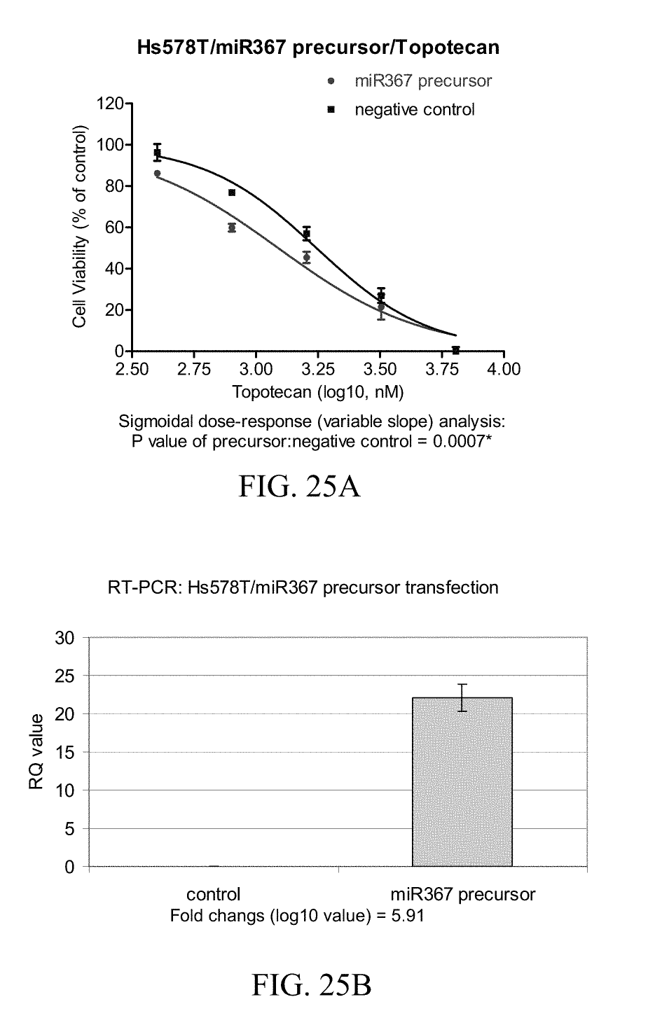

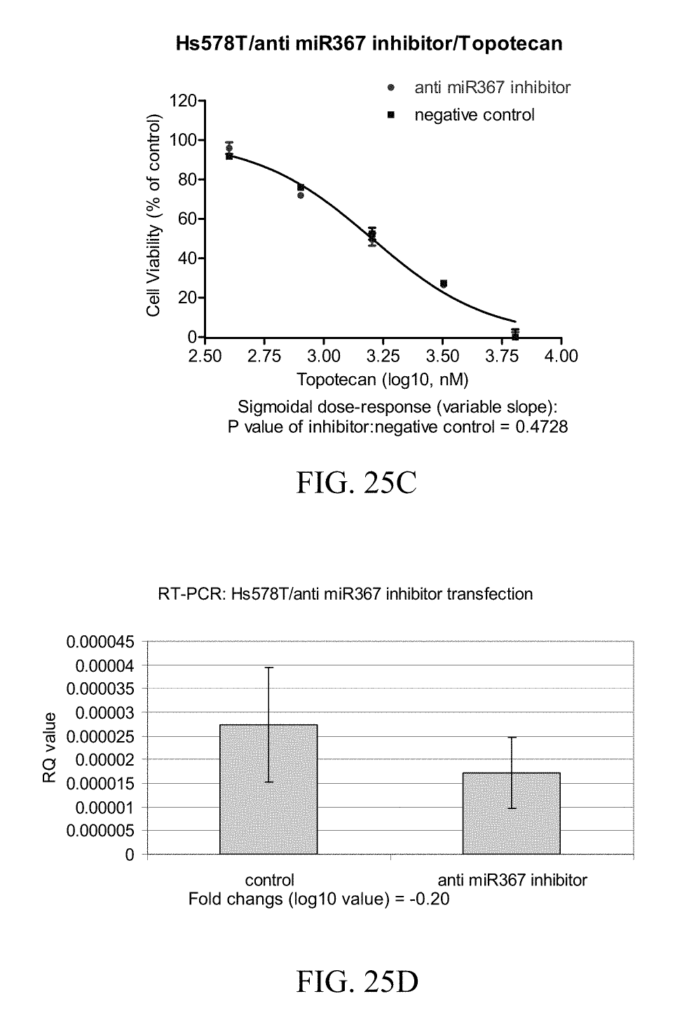

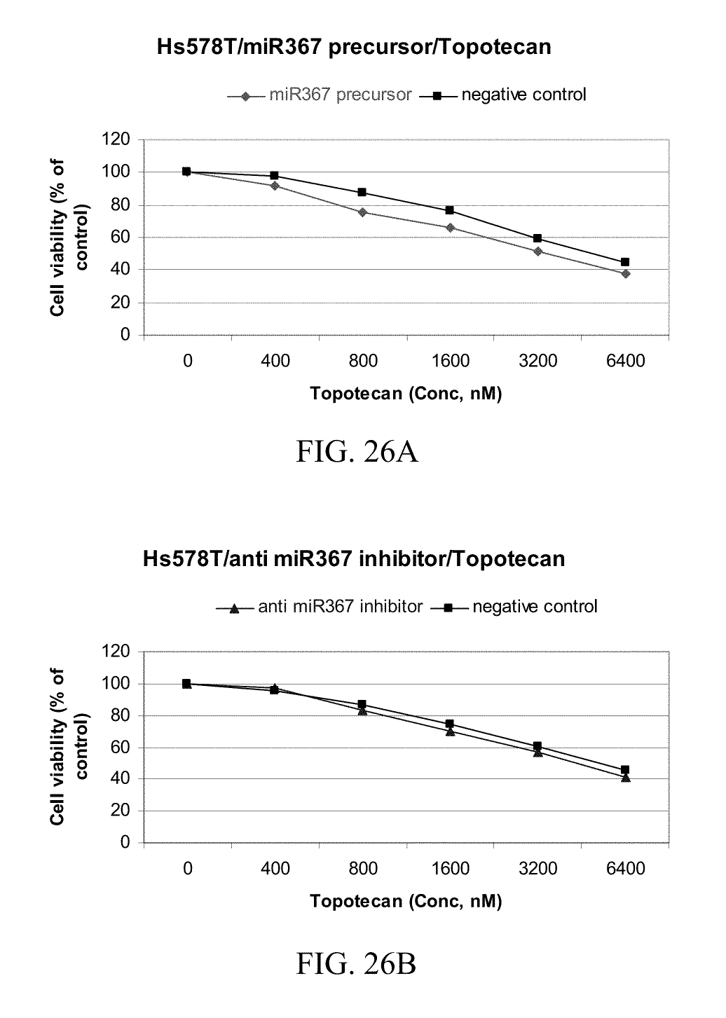

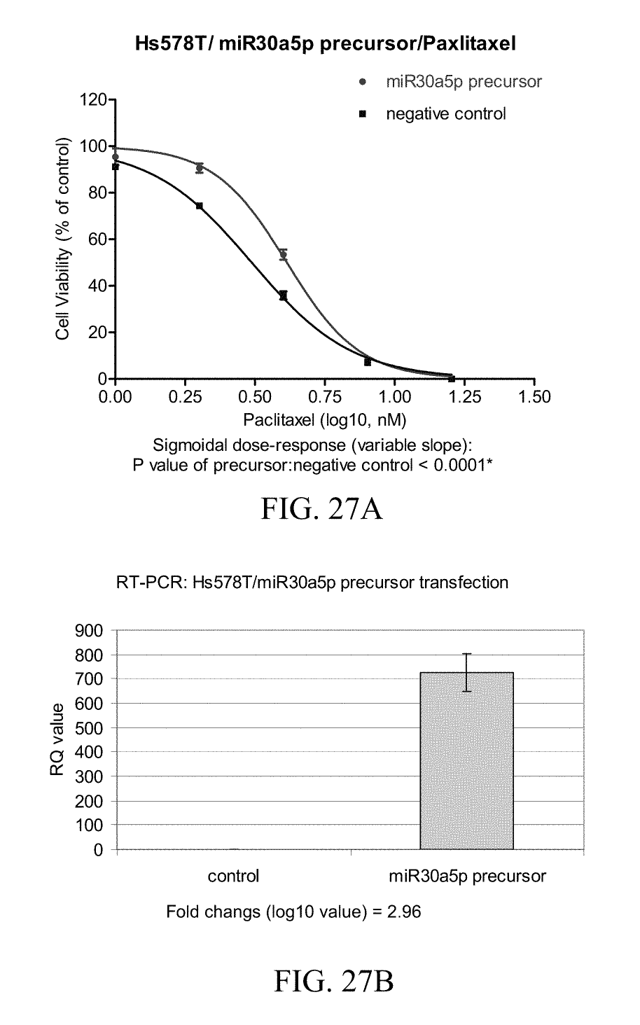

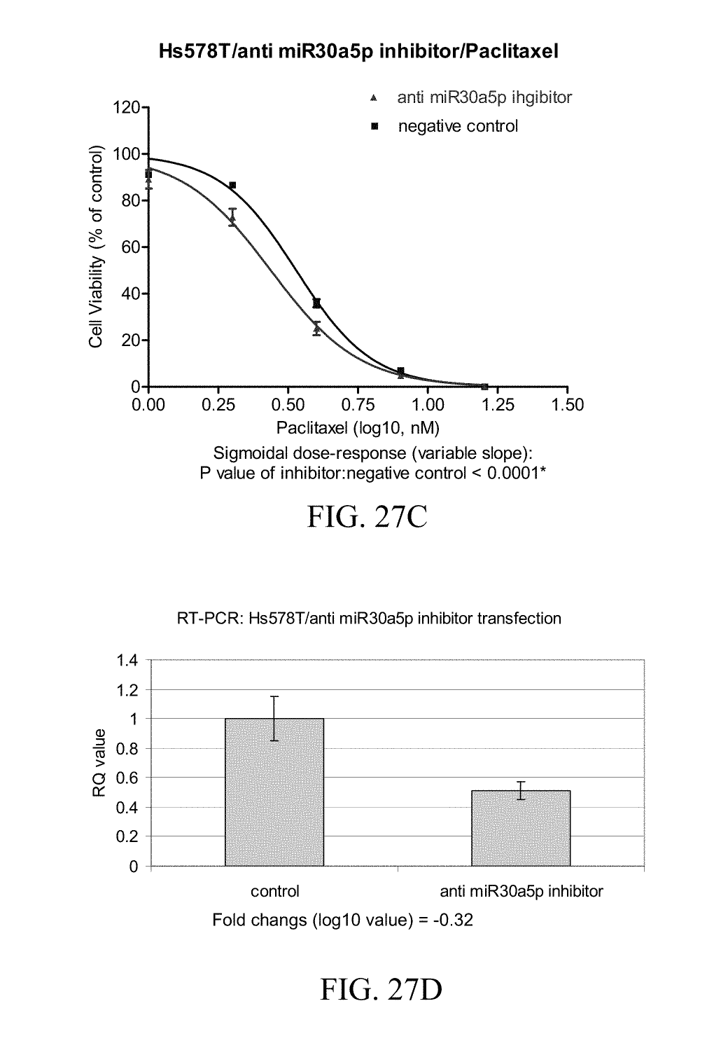

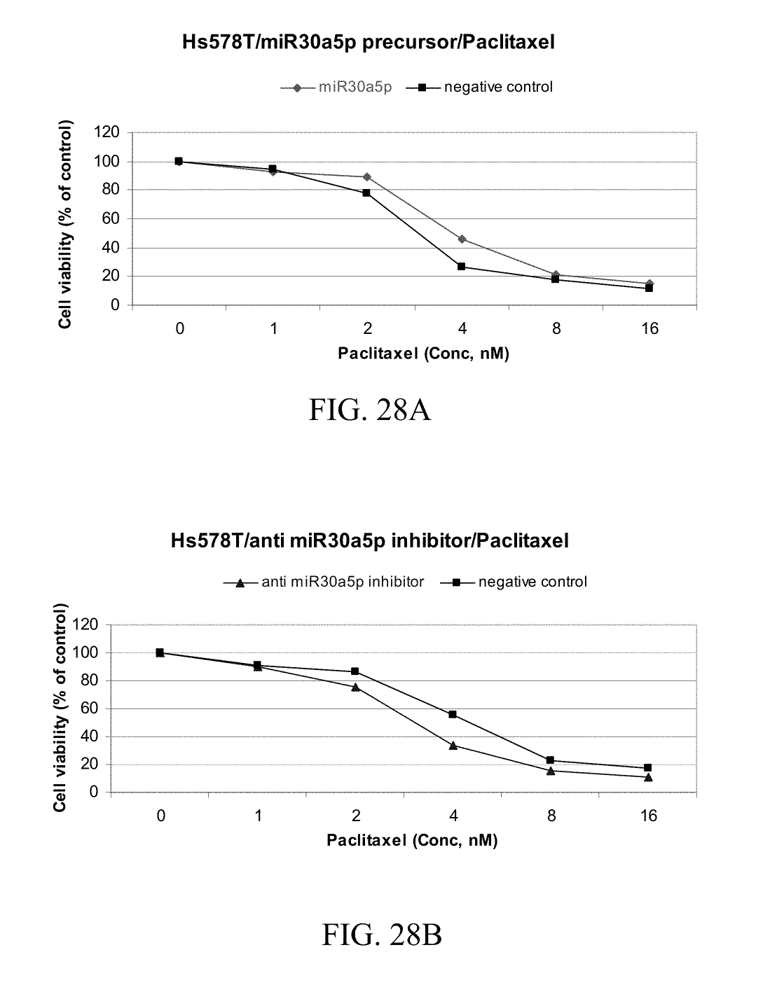

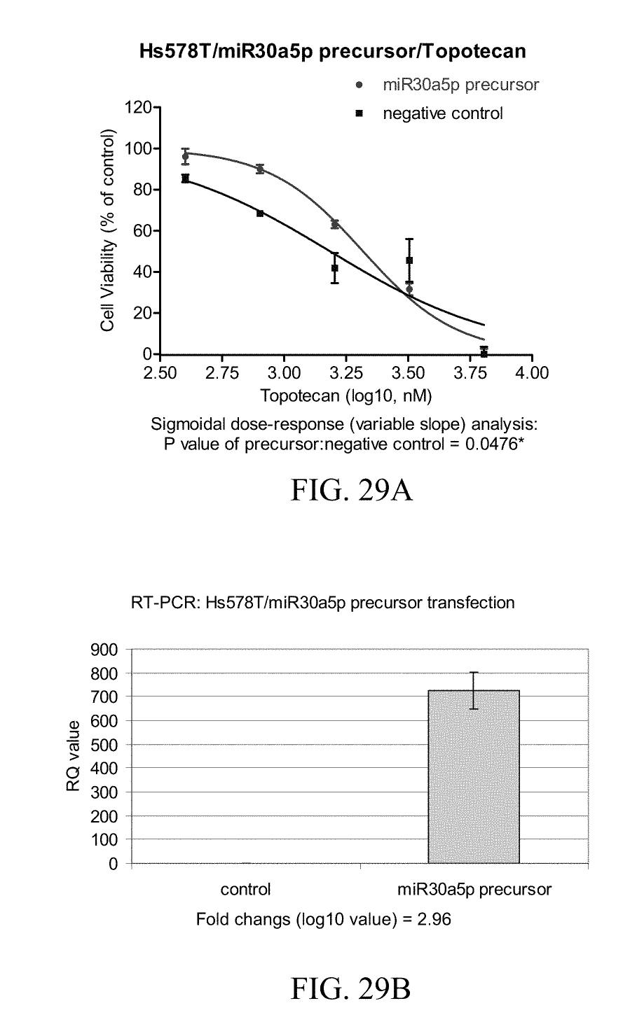

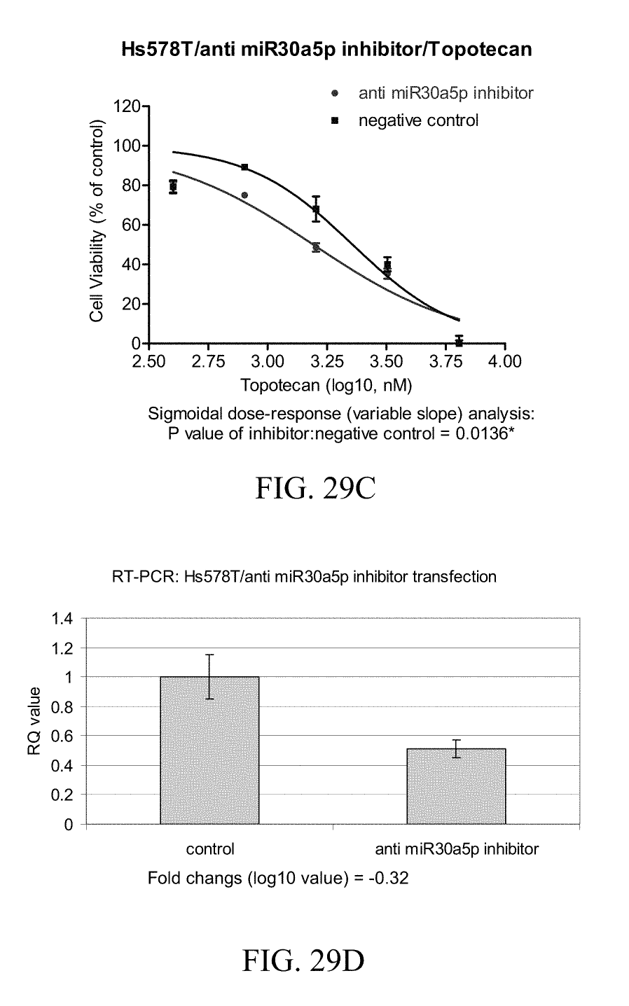

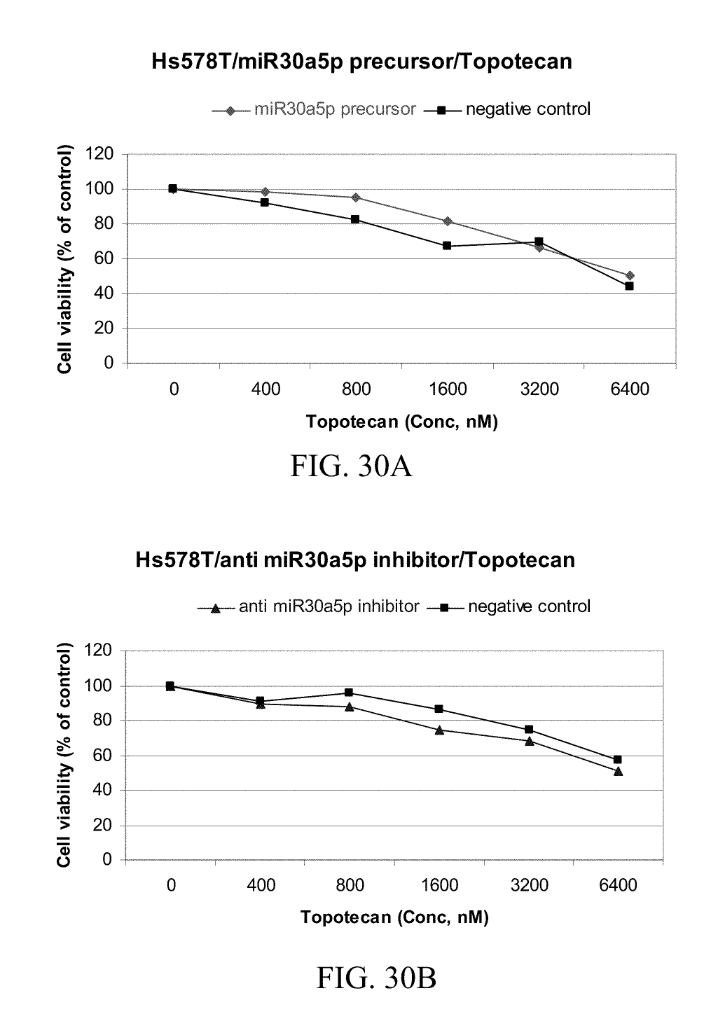

The effects of miRNA modulation on topotecan-induced and paclitaxel-induced cell death and growth arrest were evaluated using the CellTiter-Glo™ luminescent cell viability assay. Twenty-four hours after transfection, cells were subject to 48 hours of topotecan or paclitaxel treatment for 72 hours, and cell viability was analyzed. A real-time relative quantity RT-PCR was used to validate successful miRNA transfection. Fold changes=Log10 (2̂-ΔΔC_{T}). Results are shown in Tables 2 and 3, and FIGS. 5A-30B.

The efficacy of cancer treatment is frequently limited by intrinsic and acquired resistance to chemotherapy, and despite progress in delineating the molecular determinants of cancer chemo-response, a comprehensive understanding of the factors that underlie drug resistance remains elusive. Evidence is accumulating to support a role for miRNAs in human cancer [4, 21, 22], specifically associated with the development and/or progression of breast, colon, hepatocellular, stomach, prostate, pancreatic, lung, ovarian and endometrial cancers [23-28]. Moreover, recent data also suggests that miRNAs may influence cancer cell response to chemotherapy [11, 13, 29].

In the current study, the inventors have utilized genome-wide expression analysis integrated with publicly available chemo-sensitivity data for 40 human cancer cell lines (representing 9 different cancer cell types to seven different cytotoxic agents) to identify miRNAs that contribute to in vitro cancer cell chemo-sensitivity. Additionally, the inventors have demonstrated that such data can be utilized to identify opportunities to increase chemo-sensitivity by targeted modulation of miRNA levels. The inventors identified 16 miRNAs to be associated with sensitivity to 3 or more anti-cancer agents, suggesting some level of commonality to the miRNA determinants of responsiveness to many drugs. For example, expression of one such miRNA (miR-367) was highly correlated with sensitivity to topotecan, paclitaxel, and docetaxel. The locus of miR-367 lies just 5′ of the first coding sequence of the chromosome 4 gene HDCMA 18P [30]. To date, little is known about the biological role of miR-367, however, it has been reported that the miRNA cluster, miR302-367 is differentially expressed in embryonic stem cells (ESCs) and is regulated by ESC-associated transcription factors such as Nanog, Oct3/4, Sox2, and Rex1 [31, 32]. The inventors' analysis identified two renal cell lines that registered high and low extremes for topotecan sensitivity/miR-367 expression. Subsequent manipulation of miR-367 levels in the resistant cell line, TK-10 by transfection of the miR-367 precursor, resulted in increased sensitivity to topotecan (two tail t-test: P value=0.0134). Conversely, depletion of miR-367 by transfection of the miR-367 inhibitor in the sensitive cell line, 786-0, resulted in decreased topotecan sensitivity (two tail t-test: p value=0.05). Interestingly, the topoisomerase 1 gene (TOP1) is known to increase activity of c-Jun, a regulator of ESR1, which may be inhibited by the miR-367 target gene, KLF4 [33]. Additionally, miRDB-predicted mRNA target genes of miR-367 demonstrate an over-representation of survival/apoptosis biological pathways, including cytoplasmic/mitochondrial transport of the proapoptotic proteins Bid, Bmf and Bim (p<0.003) and also APRIL and BAFF signaling (p=0.003). It is therefore possible to speculate that miR-367 influences sensitivity of cancer cells to drugs such as topotecan, paclitaxel, and docetaxel via such mRNA pathways. In addition, the inventors' analysis suggested an association of miR-129 expression increasing resistance to topotecan, gemcitabine, and cisplatin. miR-129 has previously been reported to function as a tumor suppressor gene, and has been shown to be down-regulated by CpG island hypermethylation in endometrial cancer, resulting in loss of negative regulation of the SOX4 gene, and poor overall survival [34]. Of note, SOX4 interacts with and stabilizes the p53 protein, blocking Mdm2-mediated p53 ubiquitination and degradation, and influencing cell cycle arrest and apoptosis [35]. The inventors also found that expression of miR-155 was associated with resistance to topotecan, gemcitabine, and cisplatin. This miRNA has previously been implicated in the development of several cancer types including pancreatic, melanoma and NK-cell lymphoma/leukemia [36-38], and is known to influence expression of phosphatase and tensin homologue (PTEN) and phosphorylated AKT (ser473), which is known to modify cisplatin sensitivity [38]. Furthermore, the expression of miR-34c was also associated with resistance to docetaxel, topotecan, and gemcitabine. The miR-34 family is composed of three miRNAs (miR-34a, miR-34b and miR-34c) that are part of the p53 network and whose expression is directly induced by p53 in response to DNA damage or oncogenic stress. miR-34 targets Notch, HMGA2, and Bcl-2 genes involved in the self-renewal and survival of cancer stem cells, and has been implicated in the development of leukemia, colon, prostate, lung and other cancers [39-42]. Interestingly, restoration of miR-34 in p53-deficient human gastric cancer cells has been shown to increase sensitivity to chemotherapy [43]. miR-489, associated with chemo-response to docetaxel, paclitaxel, and gemcitabine in the inventors' analysis has previously been shown to be associated with breast cancer cell line resistance to tamoxifen [44].

Data suggests that miRNAs impact cell function by modulation of post-transcriptional activity via regulation of mRNA degradation or repression of translation and that the relationship between the level of expression of individual miRNAs and the mRNAs they target is complex [21, 45, 46]. The inventors' data supports the findings of other groups that miRNA expression is an important determinant of cancer cell response to therapy, likely via modification of multiple down-stream pathways. In this study, the use of cells derived from the NCI60 dataset has enabled us to take advantage of a significant scientific resource. The inventors' analysis of miRNA levels in these cells, integrated with existing chemosensitivity data has provided us with insights into miRNAs that may influence chemosensitivity of a broad range of cancer cell types to a range of different chemotherapeutic agents. It should be acknowledged, however, that such an approach likely preferentially identifies those miRNAs that are influential in determination of chemosensitivity across tumor types, and may not identify those miRNAs that have a cancer-specific influence on response to individual agents. Similarly, the inventors have evaluated sensitivity to a small number of agents—those commonly used in gynecologic oncology practice. Moving forward it will be possible to evaluate the same miRNA expression data for influence on sensitivity to a much larger range of agents that have been evaluated in the entire NCI60 cell panel. Though data such as these provide an important contribution to the inventors' knowledge of the underpinnings of chemosensitivity, it should be recognized that a comprehensive understanding of the biologic determinants of chemoresponse will ultimately require us to incorporate information on additional variables such as DNA sequence and copy number, mRNA expression (versus predicted mRNA targets), as well as protein levels and post-translational modifications. The inventors' present data suggest that miRNAs may be used as personalized medicine biomarkers of cancer cell response to therapy, and, moreover, may also represent viable therapeutic targets to increase cancer cell chemo-sensitivity.

TABLE 1
Human miRNAs associated with in vitro cancer cell line drug resistance

| | | | Numeric |
|---|---|---|---|
| miRNA ID | Accession number | Mature Sequence | Identifier |
| hsa_let_7a | MIMAT0000062 | UGAGGUAGUAGGUUGUAUAGUU | SEQ ID NO: 1 |
| hsa_let_7b | MIMAT0000063 | UGAGGUAGUAGGUUGUGUGGUU | SEQ ID NO: 2 |
| hsa_let_7c | MIMAT0000064 | UGAGGUAGUAGGUUGUAUGGUU | SEQ ID NO: 3 |
| hsa_let_7d | MIMAT0000065 | AGAGGUAGUAGGUUGCAUAGUU | SEQ ID NO: 4 |
| hsa_let_7e | MIMAT0000066 | UGAGGUAGGAGGUUGUAUAGUU | SEQ ID NO: 5 |
| hsa_let_7f | MIMAT0000067 | UGAGGUAGUAGAUUGUAUAGUU | SEQ ID NO: 6 |
| hsa_let_7g | MIMAT0000414 | UGAGGUAGUAGUUUGUACAGUU | SEQ ID NO: 7 |
| hsamiR_1 | | UGGAAUGUAAAGAAGUAUGUA* | SEQ ID NO: 8 |
| hsa_miR_103 | MIMAT0000101 | AGCAGCAUUGUACAGGGCUAUGA | SEQ ID NO: 9 |
| hsa_miR_106a | MIMAT0000103 | AAAAGUGCUUACAGUGCAGGUAG | SEQ ID NO: 10 |
| hsa_miR_107 | MIMAT0000104 | AGCAGCAUUGUACAGGGCUAUCA | SEQ ID NO: 11 |
| hsa_miR_10a | MIMAT0000253 | UACCCUGUAGAUCCGAAUUUGUG | SEQ ID NO: 12 |
| hsa_miR_10b | MIMAT0000254 | UACCCUGUAGAACCGAAUUUGUG | SEQ ID NO: 13 |
| hsa_miR_124a | | UUAAGGCACGCGGUGAAUGCCA* | SEQ ID NO: 14 |
| hsa_miR_125 a | | UCCCUGAGACCCUUUAACCUGUG* | SEQ ID NO: 15 |
| hsa_miR_126 | MIMAT0000445 | UCGUACCGUGAGUAAUAAUGCG | SEQ ID NO: 16 |
| hsa_miR_126_AS | | CGCGUACCAAAAGUAAUAAUG* | SEQ ID NO: 17 |
| hsa_miR_129 | MIMAT0000242 | CUUUUUGCGGUCUGGGCUUGC | SEQ ID NO: 18 |
| hsa_miR_13 Oa | MIMAT0000425 | CAGUGCAAUGUUAAAAGGGCAU | SEQ ID NO: 19 |
| hsa_miR_130b | MIMAT0000691 | CAGUGCAAUGAUGAAAGGGCAU | SEQ ID NO: 20 |
| hsa_miR_133b | MIMAT0000770 | UUUGGUCCCCUUCAACCAGCUA | SEQ ID NO: 21 |
| hsa_miR_134 | MIMAT0000447 | UGUGACUGGUUGACCAGAGGGG | SEQ ID NO: 22 |
| hsa_miR_138 | MIMAT0000430 | AGCUGGUGUUGUGAAUCAGGCCG | SEQ ID NO: 23 |
| hsa_miR_141 | MIMAT0000432 | UAACACUGUCUGGUAAAGAUGG | SEQ ID NO: 24 |
| hsa_miR_142_3p | MIMAT0000434 | UGUAGUGUUUCCUACUUUAUGGA | SEQ ID NO: 25 |
| hsa_miR_142_5p | MIMAT0000433 | CAUAAAGUAGAAAGCACUACU | SEQ ID NO: 26 |
| hsa_miR_146a | MIMAT0000449 | UGAGAACUGAAUUCCAUGGGUU | SEQ ID NO: 27 |
| hsa_miR_146b | MIMAT0002809 | UGAGAACUGAAUUCCAUAGGCU | SEQ ID NO: 28 |
| | MIMAT0004766 | UGCCCUGUGGACUCAGUUCUGG | SEQ ID NO: 29 |
| hsa_miR_147 | MIMAT0000251 | GUGUGUGGAAAUGCUUCUGC | SEQ ID NO: 30 |
| hsa_miR_148a | MIMAT0000243 | UCAGUGCACUACAGAACUUUGU | SEQ ID NO: 31 |
| hsa_miR_148b | MIMAT0000759 | UCAGUGCAUCACAGAACUUUGU | SEQ ID NO: 32 |
| hsa_miR_149 | MIMAT0000450 | UCUGGCUCCGUGUCUUCACUCCC | SEQ ID NO: 33 |
| hsa_miR_151 | MIMAT0004697 | UCGAGGAGCUCACAGUCUAGU | SEQ ID NO: 34 |
| | MIMAT0000757 | CUAGACUGAAGCUCCUUGAGG | SEQ ID NO: 35 |
| hsa_miR_153 | MIMAT0000439 | UUGCAUAGUCACAAAAGUGAUC | SEQ ID NO: 36 |
| hsa_miR_154 | MIMAT0000452 | UAGGUUAUCCGUGUUGCCUUCG | SEQ ID NO: 37 |
| hsa_miR_154* | MIMAT0000453 | AAUCAUACACGGUUGACCUAUU | SEQ ID NO: 38 |
| hsa_miR_155 | MIMAT0000646 | UUAAUGCUAAUCGUGAUAGGGGU | SEQ ID NO: 39 |
| hsa_miR_15a | MIMAT0000068 | UAGCAGCACAUAAUGGUUUGUG | SEQ ID NO: 40 |
| hsa_miR_15b | MIMAT0000417 | UAGCAGCACAUCAUGGUUUACA | SEQ ID NO: 41 |
| hsa_miR_17_3p | | CAAAGUGCUUACAGUGCAGGUAGU* | SEQ ID NO: 42 |
| hsa_miR_17_5p | | ACUGCAGUGAAGGCACUUGU* | SEQ ID NO: 43 |
| hsa_miR_181a | MIMAT0000256 | AACAUUCAACGCUGUCGGUGAGU | SEQ ID NO: 44 |
| hsa_miR_181b | MIMAT0000257 | AACAUUCAUUGCUGUCGGUGGGU | SEQ ID NO: 45 |
| hsa_miR_181c | MIMAT0000258 | AACAUUCAACCUGUCGGUGAGU | SEQ ID NO: 46 |
| hsa_miR_181d | MIMAT0002821 | AACAUUCAUUGUUGUCGGUGGGU | SEQ ID NO: 47 |
| hsa_miR_182 | MIMAT0000259 | UUUGGCAAUGGUAGAACUCACACU | SEQ ID NO: 48 |
| hsa_miR_183 | MIMAT0000261 | UAUGGCACUGGUAGAAUUCACU | SEQ ID NO: 49 |
| hsa_miR_184 | MIMAT0000454 | UGGACGGAGAACUGAUAAGGGU | SEQ ID NO: 50 |
| hsa_miR_18a | MIMAT0000072 | UAAGGUGCAUCUAGUGCAGAUAG | SEQ ID NO: 51 |
| hsa_miR_190 | MIMAT0000458 | UGAUAUGUUUGAUAUAUUAGGU | SEQ ID NO: 52 |
| hsa_miR_192 | MIMAT0000222 | CUGACCUAUGAAUUGACAGCC | SEQ ID NO: 53 |
| hsa_miR_193b | MIMAT0002819 | AACUGGCCCUCAAAGUCCCGCU | SEQ ID NO: 54 |
| hsa_miR_195 | MIMAT0000461 | UAGCAGCACAGAAAUAUUGGC | SEQ ID NO: 55 |
| hsa_miR_196a | MIMAT0000226 | UAGGUAGUUUCAUGUUGUUGGG | SEQ ID NO: 56 |
| hsa_miR_199a_AS | | GAACAGGUAGUCUGAACACUGGG* | SEQ ID NO: 57 |
| hsa_miR_199b | MIMAT0000263 | CCCAGUGUUUAGACUAUCUGUUC | SEQ ID NO: 58 |
| hsa_miR_19a | MIMAT0000073 | UGUGCAAAUCUAUGCAAAACUGA | SEQ ID NO: 59 |
| hsa_miR_19b | MIMAT0000074 | UGUGCAAAUCCAUGCAAAACUGA | SEQ ID NO: 60 |
| hsa_miR_200a | MIMAT0000682 | UAACACUGUCUGGUAACGAUGU | SEQ ID NO: 61 |
| hsa_miR_200b | MIMAT0000318 | UAAUACUGCCUGGUAAUGAUGA | SEQ ID NO: 62 |
| hsa_miR_200c | MIMAT0000617 | UAAUACUGCCGGGUAAUGAUGGA | SEQ ID NO: 63 |
| hsa_miR_205 | MIMAT0000266 | UCCUUCAUUCCACCGGAGUCUG | SEQ ID NO: 64 |
| hsa_miR_21 | MIMAT0000076 | UAGCUUAUCAGACUGAUGUUGA | SEQ ID NO: 65 |
| hsa_miR_213 | | ACCAUCGACCGUUGAUUGUACC* | SEQ ID NO: 66 |
| hsa_miR_215 | MIMAT0000272 | AUGACCUAUGAAUUGACAGAC | SEQ ID NO: 67 |
| hsa_miR_216 | | UAAUCUCAGCUGGCAACUGUG* | SEQ ID NO: 68 |
| hsa_miR_218 | MIMAT0000275 | UUGUGCUUGAUCUAACCAUGU | SEQ ID NO: 69 |
| hsa_miR_219 | MIMAT0000276 | UGAUUGUCCAAACGCAAUUCU | SEQ ID NO: 70 |
| hsa_miR_221 | MIMAT0000278 | AGCUACAUUGUCUGCUGGGUUUC | SEQ ID NO: 71 |
| hsa_miR_222 | MIMAT0000279 | AGCUACAUCUGGCUACUGGGU | SEQ ID NO: 72 |
| hsa_miR_224 | MIMAT0000281 | CAAGUCACUAGUGGUUCCGUU | SEQ ID NO: 73 |
| hsa_miR_25 | MIMAT0000081 | CAUUGCACUUGUCUCGGUCUGA | SEQ ID NO: 74 |
| hsa_miR_26b | MIMAT0000083 | UUCAAGUAAUUCAGGAUAGGU | SEQ ID NO: 75 |
| hsa_miR_27a | MIMAT0000084 | UUCACAGUGGCUAAGUUCCGC | SEQ ID NO: 76 |
| hsa_miR_28 | MIMAT0000085 | AAGGAGCUCACAGUCUAUUGAG | SEQ ID NO: 77 |
| hsa_miR_296 | MIMAT0000690 | AGGGCCCCCCCUCAAUCCUGU | SEQ ID NO: 78 |
| hsa_miR_29a | MIMAT0000086 | UAGCACCAUCUGAAAUCGGUUA | SEQ ID NO: 79 |
| hsa_miR_29c | MIMAT0000681 | UAGCACCAUUUGAAAUCGGUUA | SEQ ID NO: 80 |
| hsa_miR_302a | MIMAT0000684 | UAAGUGCUUCCAUGUUUUGGUGA | SEQ ID NO: 81 |
| hsa_miR_302a* | MIMAT0000683 | ACUUAAACGUGGAUGUACUUGCU | SEQ ID NO: 82 |
| hsa_miR_302c | MIMAT0000717 | UAAGUGCUUCCAUGUUUCAGUGG | SEQ ID NO: 83 |
| hsa_miR_30a_3p | | CUUUCAGUCGGAUGUUUGCAGC** | SEQ ID NO: 84 |
| hsa_miR_30a_5p | | UGUAAACAUCCUCGACUGGAAG** | SEQ ID NO: 85 |
| hsa_miR_30b | MIMAT0000420 | UGUAAACAUCCUACACUCAGCU | SEQ ID NO: 86 |
| hsa_miR_30c | MIMAT0000244 | UGUAAACAUCCUACACUCUCAGC | SEQ ID NO: 87 |
| hsa_miR_30d | MIMAT0000245 | UGUAAACAUCCCCGACUGGAAG | SEQ ID NO: 88 |
| hsa_miR_30e_5p | MIMAT0000692 | UGUAAACAUCCUUGACUGGAAG | SEQ ID NO: 89 |
| hsa_miR_31 | MIMAT0000089 | AGGCAAGAUGCUGGCAUAGCU | SEQ ID NO: 90 |
| hsa_miR_32 | MIMAT0000090 | UAUUGCACAUUACUAAGUUGCA | SEQ ID NO: 91 |
| hsa_miR_324_3p | MIMAT0000762 | ACUGCCCCAGGUGCUGCUGG | SEQ ID NO: 92 |
| hsa_miR_324_5p | MIMAT0000761 | CGCAUCCCCUAGGGCAUUGGUGU | SEQ ID NO: 93 |
| hsa_miR_337 | | UCCAGCUCCUAUAUGAUGCCUUU* | SEQ ID NO: 94 |
| hsa_miR_338 | | UCCAGCAUCAGUGAUUUUGUUGA* | SEQ ID NO: 95 |
| hsa_miR_339 | | UCCCUGUCCUCCAGGAGCUCA* | SEQ ID NO: 96 |
| hsa_miR_340 | MIMAT0004692 | UUAUAAAGCAAUGAGACUGAUU | SEQ ID NO: 97 |
| hsa_miR_342 | | UCUCACACAGAAAUCGCACCCGUC* | SEQ ID NO: 98 |
| hsa_miR_34c | MIMAT0000686 | AGGCAGUGUAGUUAGCUGAUUGC | SEQ ID NO: 99 |
| hsa_miR_361 | MIMAT0000703 | UUAUCAGAAUCUCCAGGGGUAC | SEQ ID NO: 100 |
| hsa_miR_367 | MIMAT0000719 | AAUUGCACUUUAGCAAUGGUGA | SEQ ID NO: 101 |
| hsa_miR_370 | MIMAT0000722 | GCCUGCUGGGGUGGAACCUGGU | SEQ ID NO: 102 |
| hsa_miR_373 | MIMAT0000726 | GAAGUGCUUCGAUUUUGGGGUGU | SEQ ID NO: 103 |
| hsa_miR_373* | MIMAT0000725 | ACUCAAAAUGGGGGCGCUUUCC | SEQ ID NO: 104 |
| hsa_miR_374 | | UUAUAAUACAACCUGAUAAGUG* | SEQ ID NO: 105 |
| hsa_miR_376b | MIMAT0002172 | AUCAUAGAGGAAAAUCCAUGUU | SEQ ID NO: 106 |
| hsa_miR_377 | MIMAT0000730 | AUCACACAAAGGCAACUUUUGU | SEQ ID NO: 107 |
| hsa_miR_378 | MIMAT0000732 | ACUGGACUUGGAGUCAGAAGG | SEQ ID NO: 108 |
| hsa_miR_379 | MIMAT0000733 | UGGUAGACUAUGGAACGUAGG | SEQ ID NO: 109 |
| hsa_miR_380_5p | | UGGUUGACCAUAGAACAUGCGC* | SEQ ID NO: 110 |
| hsa_miR_381 | MIMAT0000736 | UAUACAAGGGCAAGCUCUCUGU | SEQ ID NO: 111 |
| hsa_miR_382 | MIMAT0000737 | GAAGUUGUUCGUGGUGGAUUCG | SEQ ID NO: 112 |
| hsa_miR_383 | MIMAT0000738 | AGAUCAGAAGGUGAUUGUGGCU | SEQ ID NO: 113 |
| hsa_miR_384 | MIMAT0001075 | AUUCCUAGAAAUUGUUCAUA | SEQ ID NO: 114 |
| hsa_miR_410 | MIMAT0002171 | AAUAUAACACAGAUGGCCUGU | SEQ ID NO: 115 |
| hsa_miR_422a | MIMAT0001339 | ACUGGACUUAGGGUCAGAAGGC | SEQ ID NO: 116 |
| hsa_miR_423 | | AGCUCGGUCUGAGGCCCCUCAG* | SEQ ID NO: 117 |
| hsa_miR_425 | MIMAT0003393 | AAUGACACGAUCACUCCCGUUGA | SEQ ID NO: 118 |
| hsa_miR_429 | MIMAT0001536 | UAAUACUGUCUGGUAAAACCGU | SEQ ID NO: 119 |
| hsa_miR_432 | MIMAT0002814 | UCUUGGAGUAGGUCAUUGGGUGG | SEQ ID NO: 120 |
| hsa_miR_432* | MIMAT0002815 | CUGGAUGGCUCCUCCAUGUCU | SEQ ID NO: 121 |
| hsa_miR_432_AS | | CCACCCAAUGACCUACUCCAAGA* | SEQ ID NO: 122 |
| hsa_miR_452_AS | | GUCUCAGUUUCCUCUGCAAACA* | SEQ ID NO: 123 |
| hsa_miR_485_3p | MIMAT0002176 | GUCAUACACGGCUCUCCUCUCU | SEQ ID NO: 124 |
| hsa_miR_488 | MIMAT0004763 | UUGAAAGGCUAUUUCUUGGUC | SEQ ID NO: 125 |
| hsa_miR_489 | MIMAT0002805 | GUGACAUCACAUAUACGGCAGC | SEQ ID NO: 126 |
| hsa_miR_491 | | AGUGGGGAACCCUUCCAUGAGG* | SEQ ID NO: 127 |
| hsa_miR_494 | MIMAT0002816 | UGAAACAUACACGGGAAACCUC | SEQ ID NO: 128 |
| hsa_miR_498 | MIMAT0002824 | UUUCAAGCCAGGGGGCGUUUUUC | SEQ ID NO: 129 |
| hsa_miR_504 | MIMAT0002875 | AGACCCUGGUCUGCACUCUAUC | SEQ ID NO: 130 |
| hsa_miR_505 | MIMAT0002876 | CGUCAACACUUGCUGGUUUCCU | SEQ ID NO: 131 |
| hsa_miR_508 | | UGAUUGUAGCCUUUUGGAGUAGA* | SEQ ID NO: 132 |
| hsa_miR_509 | | UGAUUGGUACGUCUGUGGGUAGA* | SEQ ID NO: 133 |
| hsa_miR_512_5p | MIMAT0002822 | CACUCAGCCUUGAGGGCACUUUC | SEQ ID NO: 134 |
| hsa_miR_515_3p | MIMAT0002827 | GAGUGCCUUCUUUUGGAGCGUU | SEQ ID NO: 135 |
| hsa_miR_515_5p | MIMAT0002826 | UUCUCCAAAAGAAAGCACUUUCUG | SEQ ID NO: 136 |
| hsa_miR_516_3p | | UGCUUCCUUUCAGAGGGU* | SEQ ID NO: 137 |
| hsa_miR_517* | MIMAT0002851 | CCUCUAGAUGGAAGCACUGUCU | SEQ ID NO: 138 |
| hsa_miR_518a | | AAAGCGCUUCCCUUUGCUGGA* | SEQ ID NO: 139 |
| hsa_miR_518c* | MIMAT0002847 | UCUCUGGAGGGAAGCACUUUCUG | SEQ ID NO: 140 |
| hsa_miR_518e | MIMAT0002861 | AAAGCGCUUCCCUUCAGAGUG | SEQ ID NO: 141 |
| hsa_miR_518f | MIMAT0002842 | GAAAGCGCUUCUCUUUAGAGG | SEQ ID NO: 142 |
| hsa_miR_520a_AS | | ACAGUCCAAAGGGAAGCACUUU* | SEQ ID NO: 143 |
| hsa_miR_520b | MIMAT0002843 | AAAGUGCUUCCUUUUAGAGGG | SEQ ID NO: 144 |
| hsa_miR_520c | | AAAGUGCUUCCUUUUAGAGGGU* | SEQ ID NO: 145 |
| hsa_miR_521 | MIMAT0002854 | AACGCACUUCCCUUUAGAGUGU | SEQ ID NO: 146 |
| hsa_miR_523 | MIMAT0002840 | GAACGCGCUUCCCUAUAGAGGGU | SEQ ID NO: 147 |
| hsa_miR_524 | MIMAT0002850 | GAAGGCGCUUCCCUUUGGAGU* | SEQ ID NO: 148 |
| hsa_miR_525 | MIMAT0002838 | CUCCAGAGGGAUGCACUUUCU* | SEQ ID NO: 149 |
| hsa_miR_526a | MIMAT0002845 | CUCUAGAGGGAAGCACUUUCUG | SEQ ID NO: 150 |
| hsa_miR_7 | MIMAT0000252 | UGGAAGACUAGUGAUUUUGUUGU | SEQ ID NO: 151 |
| hsa_miR_92 | | UAUUGCACUUGUCCCGGCCUG* | SEQ ID NO: 152 |
| hsa_miR_93 | MIMAT0000093 | CAAAGUGCUGUUCGUGCAGGUAG | SEQ ID NO: 153 |
| hsa_miR_95 | MIMAT0000094 | UUCAACGGGUAUUUAUUGAGCA | SEQ ID NO: 154 |
| hsa_miR_98 | MIMAT0000096 | UGAGGUAGUAAGUUGUAUUGUU | SEQ ID NO: 155 |
| hsa_miR_99a | MIMAT0000097 | AACCCGUAGAUCCGAUCUUGUG | SEQ ID NO: 156 |
| hsa_miR_99b | MIMAT0000689 | CACCCGUAGAACCGACCUUGCG | SEQ ID NO: 157 |

miRNAs sequence data source:
miRNA Registry-miRBase--http://www.mirbase.org/
*Patentdocs--http://www.faqs.org/patents/app/20090186348.

TABLE 2
Comparison of logEC50 between miRNAs transfected cell
lines and controls (*P value <0.05)

| | | Anti | | Anti | | Anti |
|---|---|---|---|---|---|---|
| | miR302b | miR302b | miR367 | miR367 | miR30a5p | miR30a5p |
| | precursor | inhibitor | precursor | inhibitor | precursor | inhibitor |
| | (p value) | (p value) | (p value) | (p value) | (p value) | (p value) |
| ChicisR- | | | | | 0.8072 | 0.0012* |
| Paclitaxel | | | | | | |
| ChicsR- | 0.0003* | 0.0008* | | | <0.0001* | <0.0001* |
| Topotecan | | | | | | |
| OVCAR4- | <0.0001* | <0.0001* | | | 0.0278* | <0.0001* |
| Paclitaxel | | | | | | |
| OVCAR4- | <0.0001* | 0.4847 | | | | |
| Topotecan | | | | | | |
| SKOV4- | | | | | <0.0001* | 0.4339 |
| Paclitaxel | | | | | | |
| PA1-Paclitaxel | | | | <0.0001* | | |
| MCF-7- | | | | | 0.1864 | 0.0165* |
| Topotecan | | | | | | |
| Hs578T- | | | <0.0001* | <0.0001* | <0.0001* | <0.0001* |
| Paclitaxel | | | | | | |
| Hs578T- | | | 0.0007* | 0.4728 | 0.00476* | 0.00136* |
| Topotecan | | | | | | |

*means p value significant difference between transfected and control groups
miR302b and miR367: there is a lower expression in drug-resistant cell lines
miR30a5p: there is a higher expression in drug-resistant cell lines

TABLE 3
Cell lines response to Topotecan/Paclitaxel (based on IC50)

| | Topotecan | Paclitaxel |
|---|---|---|
| ChicisR | sensi | Sensi |
| OVCAR4 | resis | resis |
| SKOV4 | sensi | sensi |
| PA1 | resis | sensi |
| MCF-7 | resis | sensi |
| Hs578T | resis | sensi |

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

The following publications, which are cited above, are hereby incorporated by reference:
[1] Lim L P, Lau N C, Garrett-Engele P, Grimson A, Schelter J M, Castle J, Bartel D P, Linsley P S, Johnson J M. Microarray analysis shows that some microRNAs downregulate large numbers of target mRNAs. Nature 2005; 433: 769-73.
[2] Cummins J M, He Y, Leary R J, Pagliarini R, Diaz L A, Jr., Sjoblom T, Barad O, Bentwich Z, Szafranska A E, Labourier E, Raymond C K, Roberts B S, Juhl H, Kinzler K W, Vogelstein B, Velculescu V E. The colorectal microRNAome. Proc Natl Acad Sci USA 2006; 103: 3687-92.
[3] He L, Thomson J M, Hemann M T, Hernando-Monge E, Mu D, Goodson S, Powers S, Cordon-Cardo C, Lowe S W, Hannon G J, Hammond S M. A microRNA polycistron as a potential human oncogene. Nature 2005; 435: 828-33.
[4] Lu J, Getz G, Miska E A, Alvarez-Saavedra E, Lamb J, Peck D, Sweet-Cordero A, Ebert B L, Mak R H, Ferrando A A, Downing J R, Jacks T, Horvitz H R, Golub T R. MicroRNA expression profiles classify human cancers. Nature 12005; 435: 834-8.
[5] Chen C Z. MicroRNAs as oncogenes and tumor suppressors. N Engl J Med 2005; 353: 1768-71.
[6] Hwang H W, Mendell J T. MicroRNAs in cell proliferation, cell death, and tumorigenesis. Br J Cancer 2006; 94: 776-80.
[7] Johnson S M, Grosshans H, Shingara J, Byrom M, Jarvis R, Cheng A, Labourier E, Reinert K L, Brown D, Slack F J. RAS is regulated by the let-7 microRNA family. Cell 2005; 120: 635-47.
[8] Corney D C, Flesken-Nikitin A, Godwin A K, Wang W, Nikitin A Y. MicroRNA-34b and MicroRNA-34c are targets of p53 and cooperate in control of cell proliferation and adhesion-independent growth. Cancer Res 2007; 67: 8433-8.
[9] Chang T C, Wentzel E A, Kent O A, Ramachandran K, Mullendore M, Lee K H, Feldmann G, Yamakuchi M, Ferlito M, Lowenstein C J, Arking D E, Beer M A, Maitra A, Mendell J T. Transactivation of miR-34a by p53 broadly influences gene expression and promotes apoptosis. Mol Cell 2007; 26: 745-52.
[10] Debernardi S, Skoulakis S, Molloy G, Chaplin T, Dixon-McIver A, Young B D. MicroRNA miR-181a correlates with morphological sub-class of acute myeloid leukaemia and the expression of its target genes in global genome-wide analysis. Leukemia 2007; 21: 912-6.
[11] Blower P E, Chung J H, Verducci J S, Lin S, Park J K, Dai Z, Liu C G, Schmittgen T D, Reinhold W C, Croce C M, Weinstein J N, Sadee W. MicroRNAs modulate the chemosensitivity of tumor cells. Mol Cancer Ther 2008; 7: 1-9.
[12] Zheng T, Wang J, Chen X, Liu L. Role of microRNA in anticancer drug resistance. Int J Cancer 126: 2-10.
[13] Boren T, Xiong Y, Hakam A, Wenham R, Apte S, Chan G, Kamath S G, Chen D T, Dressman H, Lancaster J M. MicroRNAs and their target messenger RNAs associated with ovarian cancer response to chemotherapy. Gynecol Oncol 2009; 113: 249-55.
[14] Wu X, Xiao H. miRNAs modulate the drug response of tumor cells. Sci China C Life Sci 2009; 52: 797-801.
[15] Barbarotto E, and Calin, A. G. MicroRNAs and drug resistance. In: Drug resistance in cancer cells. Siddik HZaM, K, editor. New York: springer; 2009.
[16] Paull K D, Shoemaker R H, Hodes L, Monks A, Scudiero D A, Rubinstein L, Plowman J, Boyd M R. Display and analysis of patterns of differential activity of drugs against human tumor cell lines: development of mean graph and COMPARE algorithm. J Natl Cancer Inst 1989; 81: 1088-92.
[17] Ritchie M E, Silver J, Oshlack A, Holmes M, Diyagama D, Holloway A, Smyth G K. A comparison of background correction methods for two-colour microarrays. Bioinformatics 2007; 23: 2700-7.
[18] Smyth G K, Speed T. Normalization of cDNA microarray data. Methods 2003; 31: 265-73.
[19] Tusher V G, Tibshirani R, Chu G. Significance analysis of microarrays applied to the ionizing radiation response. Proc Natl Acad Sci USA 2001; 98: 5116-21.
[20] Wang X, El Naqa I M. Prediction of both conserved and nonconserved microRNA targets in animals. Bioinformatics 2008; 24: 325-32.
[21] Sontheimer E J, Carthew R W. Silence from within: endogenous siRNAs and miRNAs. Cell 2005; 122: 9-12.
[22] O'Donnell K A, Wentzel E A, Zeller K I, Dang C V, Mendell J T. c-Myc-regulated microRNAs modulate E2F1 expression. Nature 2005; 435: 839-43.
[23] Iorio M V, Visone R, Di Leva G, Donati V, Petrocca F, Casalini P, Taccioli C, Volinia S, Liu C G, Alder H, Calin G A, Menard S, Croce C M. MicroRNA signatures in human ovarian cancer. Cancer Res 2007; 67: 8699-707.
[24] Michael M Z, SM O C, van Holst Pellekaan N G, Young G P, James R J. Reduced accumulation of specific microRNAs in colorectal neoplasia. Mol Cancer Res 2003; 1: 882-91.
[25] Murakami Y, Yasuda T, Saigo K, Urashima T, Toyoda H, Okanoue T, Shimotohno K. Comprehensive analysis of microRNA expression patterns in hepatocellular carcinoma and non-tumorous tissues. Oncogene 2006; 25: 2537-45.
[26] Volinia S, Calin G A, Liu C G, Ambs S, Cimmino A, Petrocca F, Visone R, Iorio M, Roldo C, Ferracin M, Prueitt R L, Yanaihara N, Lanza G, Scarpa A, Vecchione A, Negrini M, Harris C C, Croce C M. A microRNA expression signature of human solid tumors defines cancer gene targets. Proc Natl Acad Sci USA 2006; 103: 2257-61.
[27] Schetter A J, Leung S Y, Sohn J J, Zanetti K A, Bowman E D, Yanaihara N, Yuen S T, Chan T L, Kwong D L, Au GK, Liu CG, Calin G A, Croce C M, Harris C C. MicroRNA expression profiles associated with prognosis and therapeutic outcome in colon adenocarcinoma. JAMA 2008; 299: 425-36.
[28] Boren T, Xiong Y, Hakam A, Wenham R, Apte S, Wei Z, Kamath S, Chen D T, Dressman H, Lancaster J M. MicroRNAs and their target messenger RNAs associated with endometrial carcinogenesis. Gynecol Oncol 2008; 110: 206-15.
[29] Blower P E, Verducci J S, Lin S, Zhou J, Chung J H, Dai Z, Liu C G, Reinhold W, Lorenzi P L, Kaldjian E P, Croce C M, Weinstein J N, Sadee W. MicroRNA expression profiles for the NCI-60 cancer cell panel. Mol Cancer Ther 2007; 6: 1483-91.
[30] Devor J E. Human microRNA polyadenylation signals. Integrated DNA Technologies. idtdna.com/support/technical/TechnicalBulletinPDF/Human_microRNA_polyadenylation_signal_usage.pdf. 2005.
[31] Barroso-delJesus A, Romero-Lopez C, Lucena-Aguilar G, Melen G J, Sanchez L, Ligero G, Berzal-Herranz A, Menendez P. Embryonic stem cell-specific miR302-367 cluster: human gene structure and functional characterization of its core promoter. Mol Cell Biol 2008; 28: 6609-19.
[32] Bar M, Wyman S K, Fritz B R, Qi J, Garg K S, Parkin R K, Kroh E M, Bendoraite A, Mitchell P S, Nelson A M, Ruzzo W L, Ware C, Radich J P, Gentleman R, Ruohola-Baker H, Tewari M. MicroRNA discovery and profiling in human embryonic stem cells by deep sequencing of small RNA libraries. Stem Cells 2008; 26: 2496-505.
[33] Mialon A, Sankinen M, Soderstrom H, Junttila T T, Holmstrom T, Koivusalo R, Papageorgiou A C, Johnson R S, Hietanen S, Elenius K, Westermarck J. DNA topoisomerase I is a cofactor for c-Jun in the regulation of epidermal growth factor receptor expression and cancer cell proliferation. Mol Cell Biol 2005; 25: 5040-51.
[34] Huang Y W, Liu J C, Deatherage D E, Luo J, Mutch D G, Goodfellow P J, Miller D S, Huang T H. Epigenetic repression of microRNA-129-2 leads to overexpression of SOX4 oncogene in endometrial cancer. Cancer Res 2009; 69: 9038-46.
[35] Pan X, Zhao J, Zhang W N, Li H Y, Mu R, Zhou T, Zhang H Y, Gong W L, Yu M, Man J H, Zhang P J, Li A L, Zhang X M. Induction of SOX4 by DNA damage is critical for p53 stabilization and function. Proc Natl Acad Sci USA 2009; 106: 3788-93.
[36] Greither T, Grochola L F, Udelnow A, Lautenschlager C, Wurl P, Taubert H. Elevated expression of microRNAs 155, 203, 210 and 222 in pancreatic tumors is associated with poorer survival. Int J Cancer 126: 73-80.
[37] Levati L, Alvino E, Pagani E, Arcelli D, Caporaso P, Bondanza S, Di Leva G, Ferracin M, Volinia S, Bonmassar E, Croce C M, D'Atri S. Altered expression of selected microRNAs in melanoma: antiproliferative and proapoptotic activity of miRNA-155. Int J Oncol 2009; 35: 393-400.
[38] Yamanaka Y, Tagawa H, Takahashi N, Watanabe A, Guo Y M, Iwamoto K, Yamashita J, Saitoh H, Kameoka Y, Shimizu N, Ichinohasama R, Sawada K. Aberrant overexpression of microRNAs activate AKT signaling via down-regulation of tumor suppressors in natural killer-cell lymphoma/leukemia. Blood 2009; 114: 3265-75.
[39] Cardinaud B, Moreilhon C, Marcet B, Robbe-Sermesant K, LeBrigand K, Mari B, Eclache V, Cymbalista F, Raynaud S, Barbry P. miR-34b/miR-34c: a regulator of TCL1 expression in 11q- chronic lymphocytic leukaemia? Leukemia 2009; 23: 2174-7.
[40] Toyota M, Suzuki H, Sasaki Y, Maruyama R, Imai K, Shinomura Y, Tokino T. Epigenetic silencing of microRNA-34b/c and B-cell translocation gene 4 is associated with CpG island methylation in colorectal cancer. Cancer Res 2008; 68: 4123-32.
[41] Gallardo E, Navarro A, Vinolas N, Marrades R M, Diaz T, Gel B, Quera A, Bandres E, Garcia-Foncillas J, Ramirez J, Monzo M. miR-34a as a prognostic marker of relapse in surgically resected non-small-cell lung cancer. Carcinogenesis 2009; 30: 1903-9.
[42] Hagman Z, Larne O, Edsjo A, Bjartell A, Ehrnstrom R A, Ulmert D, Lilja H, Ceder Y. miR-34c is down regulated in prostate cancer and exerts tumor suppressive functions. Int J Cancer.
[43] Ji Q, Hao X, Meng Y, Zhang M, Desano J, Fan D, Xu L. Restoration of tumor suppressor miR-34 inhibits human p53-mutant gastric cancer tumorspheres. BMC Cancer 2008; 8: 266.
[44] Miller T E, Ghoshal K, Ramaswamy B, Roy S, Datta J, Shapiro C L, Jacob S, Majumder S. MicroRNA-221/222 confers tamoxifen resistance in breast cancer by targeting p27Kip1. J Biol Chem 2008; 283: 29897-903.
[45] Bartel D P. MicroRNAs: genomics, biogenesis, mechanism, and function. Cell 2004; 116: 281-97.
[46] Filipowicz W, Jaskiewicz L, Kolb F A, Pillai R S. Post-transcriptional gene silencing by siRNAs and miRNAs. Curr Opin Struct Biol 2005; 15: 331-41.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between.

What is claimed is:

1. A method for preparing a microRNA (miRNA) expression profile for a cancer cell sample that is indicative of resistance or sensitivity to an anti-cancer agent, comprising: determining the level of expression of an miRNA in the sample, thereby preparing the miRNA expression profile.

2. The method of claim 1, wherein the miRNA comprises:
(a) one or more miRNAs from among SEQ ID NOs:1-157; or
(b) one or more miRNAs listed in FIGS. 4A, 4B, 4C, 4D, 4E, 4F, or 4G; or
(c) one or more miRNAs from among miR367, miR200c, miR515, miR377, miR508, miR340, miR129, miR130a, miR142_5p, miR155, miR296, miR34c, miR367, miR380_5p, miR489, miR494, and miR526.

3. A method of treating cancer in a mammalian subject, wherein the cancer has been pre-determined to express a microRNA (miRNA) at a level that is indicative of sensitivity, or lack of resistance, to an anti-cancer agent, wherein the method comprises administering a therapeutically effective amount of the anti-cancer agent to the subject.

4. The method of claim 3, wherein the miRNA comprises:
(a) one or more miRNAs from among SEQ ID NOs:1-157; or
(b) one or more miRNAs listed in FIGS. 4A, 4B, 4C, 4D, 4E, 4F, or 4G; or
(c) one or more miRNAs from among miR367, miR200c, miR515, miR377, miR508, miR340, miR129, miR130a, miR142_5p, miR155, miR296, miR34c, miR367, miR380_5p, miR489, miR494, and miR526.

5. The method of claim 3, wherein the miRNA comprises:
(a) one or more miRNAs listed in FIG. 4A, and wherein the anti-cancer agent comprises cisplatin or a cisplatin variant; or
(b) one or more miRNAs listed in FIG. 4B, and wherein the anti-cancer agent comprises docetaxel or a docetaxel variant; or
(c) one or more miRNAs listed in FIG. 4C, and wherein the anti-cancer agent comprises doxorubicin or a doxorubicin variant; or
(d) one or more miRNAs listed in FIG. 4D, and wherein the anti-cancer agent comprises gemcitabine or a gemcitabine variant; or
(e) one or more miRNAs listed in FIG. 4E, and wherein the anti-cancer agent comprises paclitaxel or a paclitaxel variant; or
(f) one or more miRNAs listed in FIG. 4F, and wherein the anti-cancer agent comprises topotecan or a topetecan variant; or
(g) one or more miRNAs listed in FIG. 4G, and wherein the anti-cancer agent comprises carboplatin or a carboplatin variant.

6. A method for predicting the response of a cancer in a mammalian subject to an anti-cancer agent, comprising: determining the microRNA (miRNA) expression profile in a cancer cell sample obtained from the subject; comparing the miRNA expression profile of the cancer cell sample to a reference miRNA expression profile associated with a predetermined sensitivity or lack of resistance to one more anti-cancer agents; and determining the predicted response of the cancer cells in the cancer cell sample to the one or more anti-cancer agents based upon the compared miRNA expression profiles, wherein the predicted response of the cancer cells in the cancer cell sample is indicative of the response of the cancer in the subject.

7. The method of claim 6, wherein the reference miRNA expression profile is the miRNA expression profile of one or more cancer cells with predetermined sensitivities to one or more anti-cancer agents.

8. The method of claim 6, wherein the miRNA comprises:
(a) one or more miRNAs from among SEQ ID NOs:1-157; or
(b) one or more miRNAs listed in FIGS. 4A, 4B, 4C, 4D, 4E, 4F, or 4G; or
(c) one or more miRNAs from among miR367, miR200c, miR515, miR377, miR508, miR340, miR129, miR130a, miR142_5p, miR155, miR296, miR34c, miR367, miR380_5p, miR489, miR494, and miR526.

9. The method of claim 6, wherein the miRNA of the miRNA expression profiles comprises:
(a) one or more miRNAs listed in FIG. 4A, and wherein the anti-cancer agent comprises cisplatin or a cisplatin variant; or
(b) one or more miRNAs listed in FIG. 4B, and wherein the anti-cancer agent comprises docetaxel or a docetaxel variant; or
(c) one or more miRNAs listed in FIG. 4C, and wherein the anti-cancer agent comprises doxorubicin or a doxorubicin variant;
(d) one or more miRNAs listed in FIG. 4D, and wherein the anti-cancer agent comprises gemcitabine or a gemcitabine variant; or
(e) one or more miRNAs listed in FIG. 4E, and wherein the anti-cancer agent comprises paclitaxel or a paclitaxel variant; or
(f) one or more miRNAs listed in FIG. 4F, and wherein the anti-cancer agent comprises topotecan or a topetecan variant; or
(g) one or more miRNAs listed in FIG. 4G, and wherein the anti-cancer agent comprises carboplatin or a carboplatin variant.


SEQUENCE LISTING (160 sequences)

SEQ ID NO: 1 - RNA, 22 nt, Homo sapiens
ugagguaguagguuguauaguu

SEQ ID NO: 2 - RNA, 22 nt, Homo sapiens
ugagguaguagguugugugguu

SEQ ID NO: 3 - RNA, 22 nt, Homo sapiens
ugagguaguagguuguaugguu

SEQ ID NO: 4 - RNA, 22 nt, Homo sapiens
agagguaguagguugcauaguu

SEQ ID NO: 5 - RNA, 22 nt, Homo sapiens
ugagguaggagguuguauaguu

SEQ ID NO: 6 - RNA, 22 nt, Homo sapiens
ugagguaguagauuguauaguu

SEQ ID NO: 7 - RNA, 22 nt, Homo sapiens
ugagguaguaguuuguacaguu

SEQ ID NO: 8 - RNA, 21 nt, Homo sapiens
uggaauguaaagaaguaugua

SEQ ID NO: 9 - RNA, 23 nt, Homo sapiens
agcagcauuguacagggcuauga

SEQ ID NO: 10 - RNA, 23 nt, Homo sapiens
aaaagugcuuacagugcagguag

SEQ ID NO: 11 - RNA, 23 nt, Homo sapiens
agcagcauuguacagggcuauca

SEQ ID NO: 12 - RNA, 23 nt, Homo sapiens
uacccuguagauccgaauuugug

SEQ ID NO: 13 - RNA, 23 nt, Homo sapiens
uacccuguagaaccgaauuugug

SEQ ID NO: 14 - RNA, 22 nt, Homo sapiens
uuaaggcacgcggugaaugcca

SEQ ID NO: 15 - RNA, 23 nt, Homo sapiens
ucccugagacccuuuaaccugug

SEQ ID NO: 16 - RNA, 22 nt, Homo sapiens
ucguaccgugaguaauaaugcg

SEQ ID NO: 17 - RNA, 21 nt, Homo sapiens
cgcguaccaaaaguaauaaug

SEQ ID NO: 18 - RNA, 21 nt, Homo sapiens
cuuuuugcggucugggcuugc

SEQ ID NO: 19 - RNA, 22 nt, Homo sapiens
cagugcaauguuaaaagggcau

SEQ ID NO: 20 - RNA, 22 nt, Homo sapiens
cagugcaaugaugaaagggcau

SEQ ID NO: 21 - RNA, 22 nt, Homo sapiens
uuugguccccuucaaccagcua

SEQ ID NO: 22 - RNA, 22 nt, Homo sapiens
ugugacugguugaccagagggg

SEQ ID NO: 23 - RNA, 23 nt, Homo sapiens
agcugguguugugaaucaggccg

SEQ ID NO: 24 - RNA, 22 nt, Homo sapiens
uaacacugucugguaaagaugg

SEQ ID NO: 25 - RNA, 23 nt, Homo sapiens
uguaguguuuccuacuuuaugga

SEQ ID NO: 26 - RNA, 21 nt, Homo sapiens
cauaaaguagaaagcacuacu

SEQ ID NO: 27 - RNA, 22 nt, Homo sapiens
ugagaacugaauuccauggguu

SEQ ID NO: 28 - RNA, 22 nt, Homo sapiens
ugagaacugaauuccauaggcu

SEQ ID NO: 29 - RNA, 22 nt, Homo sapiens
ugcccuguggacucaguucugg

SEQ ID NO: 30 - RNA, 20 nt, Homo sapiens
guguguggaaaugcuucugc

SEQ ID NO: 31 - RNA, 22 nt, Homo sapiens
ucagugcacuacagaacuuugu

SEQ ID NO: 32 - RNA, 22 nt, Homo sapiens
ucagugcaucacagaacuuugu

SEQ ID NO: 33 - RNA, 23 nt, Homo sapiens
ucuggcuccgugucuucacuccc

SEQ ID NO: 34 - RNA, 21 nt, Homo sapiens
ucgaggagcucacagucuagu

SEQ ID NO: 35 - RNA, 21 nt, Homo sapiens
cuagacugaagcuccuugagg

SEQ ID NO: 36 - RNA, 22 nt, Homo sapiens
uugcauagucacaaaagugauc

SEQ ID NO: 37 - RNA, 22 nt, Homo sapiens
uagguuauccguguugccuucg

SEQ ID NO: 38 - RNA, 22 nt, Homo sapiens
aaucauacacgguugaccuauu

SEQ ID NO: 39 - RNA, 23 nt, Homo sapiens
uuaaugcuaaucgugauaggggu

SEQ ID NO: 40 - RNA, 22 nt, Homo sapiens
uagcagcacauaaugguuugug

SEQ ID NO: 41 - RNA, 22 nt, Homo sapiens
uagcagcacaucaugguuuaca

SEQ ID NO: 42 - RNA, 24 nt, Homo sapiens
caaagugcuuacagugcagguagu

SEQ ID NO: 43 - RNA, 20 nt, Homo sapiens
acugcagugaaggcacuugu

SEQ ID NO: 44 - RNA, 23 nt, Homo sapiens
aacauucaacgcugucggugagu

SEQ ID NO: 45 - RNA, 23 nt, Homo sapiens
aacauucauugcugucggugggu

SEQ ID NO: 46 - RNA, 22 nt, Homo sapiens
aacauucaaccugucggugagu

SEQ ID NO: 47 - RNA, 23 nt, Homo sapiens
aacauucauuguugucggugggu

SEQ ID NO: 48 - RNA, 24 nt, Homo sapiens
uuuggcaaugguagaacucacacu

SEQ ID NO: 49 - RNA, 22 nt, Homo sapiens
uauggcacugguagaauucacu

SEQ ID NO: 50 - RNA, 22 nt, Homo sapiens
uggacggagaacugauaagggu

SEQ ID NO: 51 - RNA, 23 nt, Homo sapiens
uaaggugcaucuagugcagauag

SEQ ID NO: 52 - RNA, 22 nt, Homo sapiens
ugauauguuugauauauuaggu

SEQ ID NO: 53 - RNA, 21 nt, Homo sapiens
cugaccuaugaauugacagcc

SEQ ID NO: 54 - RNA, 22 nt, Homo sapiens
aacuggcccucaaagucccgcu

SEQ ID NO: 55 - RNA, 21 nt, Homo sapiens
uagcagcacagaaauauuggc

SEQ ID NO: 56 - RNA, 22 nt, Homo sapiens
uagguaguuucauguuguuggg

SEQ ID NO: 57 - RNA, 23 nt, Homo sapiens
gaacagguagucugaacacuggg

SEQ ID NO: 58 - RNA, 23 nt, Homo sapiens
cccaguguuuagacuaucuguuc

SEQ ID NO: 59 - RNA, 23 nt, Homo sapiens
ugugcaaaucuaugcaaaacuga

SEQ ID NO: 60 - RNA, 23 nt, Homo sapiens
ugugcaaauccaugcaaaacuga

SEQ ID NO: 61 - RNA, 22 nt, Homo sapiens
uaacacugucugguaacgaugu

SEQ ID NO: 62 - RNA, 22 nt, Homo sapiens
uaauacugccugguaaugauga

SEQ ID NO: 63 - RNA, 23 nt, Homo sapiens
uaauacugccggguaaugaugga

SEQ ID NO: 64 - RNA, 22 nt, Homo sapiens
uccuucauuccaccggagucug

SEQ ID NO: 65 - RNA, 22 nt, Homo sapiens
uagcuuaucagacugauguuga

SEQ ID NO: 66 - RNA, 22 nt, Homo sapiens
accaucgaccguugauuguacc

SEQ ID NO: 67 - RNA, 21 nt, Homo sapiens
augaccuaugaauugacagac

SEQ ID NO: 68 - RNA, 21 nt, Homo sapiens
uaaucucagcuggcaacugug

SEQ ID NO: 69 - RNA, 21 nt, Homo sapiens
uugugcuugaucuaaccaugu

SEQ ID NO: 70 - RNA, 21 nt, Homo sapiens
ugauuguccaaacgcaauucu

SEQ ID NO: 71 - RNA, 23 nt, Homo sapiens
agcuacauugucugcuggguuuc

SEQ ID NO: 72 - RNA, 21 nt, Homo sapiens
agcuacaucuggcuacugggu

SEQ ID NO: 73 - RNA, 21 nt, Homo sapiens
caagucacuagugguuccguu

SEQ ID NO: 74 - RNA, 22 nt, Homo sapiens
cauugcacuugucucggucuga

SEQ ID NO: 75 - RNA, 21 nt, Homo sapiens
uucaaguaauucaggauaggu

SEQ ID NO: 76 - RNA, 21 nt, Homo sapiens
uucacaguggcuaaguuccgc

SEQ ID NO: 77 - RNA, 22 nt, Homo sapiens
aaggagcucacagucuauugag

SEQ ID NO: 78 - RNA, 21 nt, Homo sapiens
agggcccccccucaauccugu

SEQ ID NO: 79 - RNA, 22 nt, Homo sapiens
uagcaccaucugaaaucgguua

SEQ ID NO: 80 - RNA, 22 nt, Homo sapiens
uagcaccauuugaaaucgguua

SEQ ID NO: 81 - RNA, 23 nt, Homo sapiens
uaagugcuuccauguuuugguga

SEQ ID NO: 82 - RNA, 23 nt, Homo sapiens
acuuaaacguggauguacuugcu

SEQ ID NO: 83 - RNA, 23 nt, Homo sapiens
uaagugcuuccauguuucagugg

SEQ ID NO: 84 - RNA, 22 nt, Homo sapiens
cuuucagucggauguuugcagc

SEQ ID NO: 85 - RNA, 22 nt, Homo sapiens
uguaaacauccucgacuggaag

SEQ ID NO: 86 - RNA, 22 nt, Homo sapiens
uguaaacauccuacacucagcu

SEQ ID NO: 87 - RNA, 23 nt, Homo sapiens
uguaaacauccuacacucucagc

SEQ ID NO: 88 - RNA, 22 nt, Homo sapiens
uguaaacauccccgacuggaag

SEQ ID NO: 89 - RNA, 22 nt, Homo sapiens
uguaaacauccuugacuggaag

SEQ ID NO: 90 - RNA, 21 nt, Homo sapiens
aggcaagaugcuggcauagcu

SEQ ID NO: 91 - RNA, 22 nt, Homo sapiens
uauugcacauuacuaaguugca

SEQ ID NO: 92 - RNA, 20 nt, Homo sapiens
acugccccaggugcugcugg

SEQ ID NO: 93 - RNA, 23 nt, Homo sapiens
cgcauccccuagggcauuggugu

SEQ ID NO: 94 - RNA, 23 nt, Homo sapiens
uccagcuccuauaugaugccuuu

SEQ ID NO: 95 - RNA, 23 nt, Homo sapiens
uccagcaucagugauuuuguuga

SEQ ID NO: 96 - RNA, 21 nt, Homo sapiens
ucccuguccuccaggagcuca

SEQ ID NO: 97 - RNA, 22 nt, Homo sapiens
uuauaaagcaaugagacugauu

SEQ ID NO: 98 - RNA, 24 nt, Homo sapiens
ucucacacagaaaucgcacccguc

SEQ ID NO: 99 - RNA, 23 nt, Homo sapiens
aggcaguguaguuagcugauugc

SEQ ID NO: 100 - RNA, 22 nt, Homo sapiens
uuaucagaaucuccagggguac

SEQ ID NO: 101 - RNA, 22 nt, Homo sapiens
aauugcacuuuagcaaugguga

SEQ ID NO: 102 - RNA, 22 nt, Homo sapiens
gccugcugggguggaaccuggu

SEQ ID NO: 103 - RNA, 23 nt, Homo sapiens
gaagugcuucgauuuuggggugu

SEQ ID NO: 104 - RNA, 22 nt, Homo sapiens
acucaaaaugggggcgcuuucc

SEQ ID NO: 105 - RNA, 22 nt, Homo sapiens
uuauaauacaaccugauaagug

SEQ ID NO: 106 - RNA, 22 nt, Homo sapiens
aucauagaggaaaauccauguu

SEQ ID NO: 107 - RNA, 22 nt, Homo sapiens
aucacacaaaggcaacuuuugu

SEQ ID NO: 108 - RNA, 21 nt, Homo sapiens
acuggacuuggagucagaagg

SEQ ID NO: 109 - RNA, 21 nt, Homo sapiens
ugguagacuauggaacguagg

SEQ ID NO: 110 - RNA, 22 nt, Homo sapiens
ugguugaccauagaacaugcgc

SEQ ID NO: 111 - RNA, 22 nt, Homo sapiens
uauacaagggcaagcucucugu

SEQ ID NO: 112 - RNA, 22 nt, Homo sapiens
gaaguuguucgugguggauucg

SEQ ID NO: 113 - RNA, 22 nt, Homo sapiens
agaucagaaggugauuguggcu

SEQ ID NO: 114 - RNA, 20 nt, Homo sapiens
auuccuagaaauuguucaua

SEQ ID NO: 115 - RNA, 21 nt, Homo sapiens
aauauaacacagauggccugu

SEQ ID NO: 116 - RNA, 22 nt, Homo sapiens
acuggacuuagggucagaaggc

SEQ ID NO: 117 - RNA, 22 nt, Homo sapiens
agcucggucugaggccccucag

SEQ ID NO: 118 - RNA, 23 nt, Homo sapiens
aaugacacgaucacucccguuga

SEQ ID NO: 119 - RNA, 22 nt, Homo sapiens
uaauacugucugguaaaaccgu

SEQ ID NO: 120 - RNA, 23 nt, Homo sapiens
ucuuggaguaggucauugggugg

SEQ ID NO: 121 - RNA, 21 nt, Homo sapiens
cuggauggcuccuccaugucu

SEQ ID NO: 122 - RNA, 23 nt, Homo sapiens
ccacccaaugaccuacuccaaga

SEQ ID NO: 123 - RNA, 22 nt, Homo sapiens
gucucaguuuccucugcaaaca

SEQ ID NO: 124 - RNA, 22 nt, Homo sapiens
gucauacacggcucuccucucu

SEQ ID NO: 125 - RNA, 21 nt, Homo sapiens
uugaaaggcuauuucuugguc

SEQ ID NO: 126 - RNA, 22 nt, Homo sapiens
gugacaucacauauacggcagc

SEQ ID NO: 127 - RNA, 22 nt, Homo sapiens
aguggggaacccuuccaugagg

SEQ ID NO: 128 - RNA, 22 nt, Homo sapiens
ugaaacauacacgggaaaccuc

SEQ ID NO: 129 - RNA, 23 nt, Homo sapiens
uuucaagccagggggcguuuuuc

SEQ ID NO: 130 - RNA, 22 nt, Homo sapiens
agacccuggucugcacucuauc

SEQ ID NO: 131 - RNA, 22 nt, Homo sapiens
cgucaacacuugcugguuuccu

SEQ ID NO: 132 - RNA, 23 nt, Homo sapiens
ugauuguagccuuuuggaguaga

SEQ ID NO: 133 - RNA, 23 nt, Homo sapiens
ugauugguacgucuguggguaga

SEQ ID NO: 134 - RNA, 23 nt, Homo sapiens
cacucagccuugagggcacuuuc

SEQ ID NO: 135 - RNA, 22 nt, Homo sapiens
gagugccuucuuuuggagcguu

SEQ ID NO: 136 - RNA, 24 nt, Homo sapiens
uucuccaaaagaaagcacuuucug

SEQ ID NO: 137 - RNA, 18 nt, Homo sapiens
ugcuuccuuucagagggu

SEQ ID NO: 138 - RNA, 22 nt, Homo sapiens
ccucuagauggaagcacugucu

SEQ ID NO: 139 - RNA, 21 nt, Homo sapiens
aaagcgcuucccuuugcugga

SEQ ID NO: 140 - RNA, 23 nt, Homo sapiens
ucucuggagggaagcacuuucug

SEQ ID NO: 141 - RNA, 21 nt, Homo sapiens
aaagcgcuucccuucagagug

SEQ ID NO: 142 - RNA, 21 nt, Homo sapiens
gaaagcgcuucucuuuagagg

SEQ ID NO: 143 - RNA, 22 nt, Homo sapiens
acaguccaaagggaagcacuuu

SEQ ID NO: 144 - RNA, 21 nt, Homo sapiens
aaagugcuuccuuuuagaggg

SEQ ID NO: 145 - RNA, 22 nt, Homo sapiens
aaagugcuuccuuuuagagggu

SEQ ID NO: 146 - RNA, 22 nt, Homo sapiens
aacgcacuucccuuuagagugu

SEQ ID NO: 147 - RNA, 23 nt, Homo sapiens
gaacgcgcuucccuauagagggu

SEQ ID NO: 148 - RNA, 21 nt, Homo sapiens
gaaggcgcuucccuuuggagu

SEQ ID NO: 149 - RNA, 21 nt, Homo sapiens
cuccagagggaugcacuuucu

SEQ ID NO: 150 - RNA, 22 nt, Homo sapiens
cucuagagggaagcacuuucug

SEQ ID NO: 151 - RNA, 23 nt, Homo sapiens
uggaagacuagugauuuuguugu

SEQ ID NO: 152 - RNA, 21 nt, Homo sapiens
uauugcacuugucccggccug

SEQ ID NO: 153 - RNA, 23 nt, Homo sapiens
caaagugcuguucgugcagguag

SEQ ID NO: 154 - RNA, 22 nt, Homo sapiens
uucaacggguauuuauugagca

SEQ ID NO: 155 - RNA, 22 nt, Homo sapiens
ugagguaguaaguuguauuguu

SEQ ID NO: 156 - RNA, 22 nt, Homo sapiens
aacccguagauccgaucuugug

SEQ ID NO: 157 - RNA, 22 nt, Homo sapiens
cacccguagaaccgaccuugcg

SEQ ID NO: 158 - RNA, 68 nt, Artificial Sequence
  Pre-miR367 precursor
ccauuacuguugcuaauaugcaacucuguugaauauaaauuggaauugcacuuuagcaauggugaugg

SEQ ID NO: 159 - RNA, 73 nt, Artificial Sequence
  pre-miR302b precursor
gcucccuucaacuuuaacauggaagugcuuucugugacuuuaaaaguaagugcuuccauguuuuaguaggagu

SEQ ID NO: 160 - RNA, 71 nt, Artificial Sequence
  pre-miR30a (miR30a-5p) precursor
gcgacuguaaacauccucgacuggaagcugugaagccacagaugggcuuucagucggauguuugcagcugc